(19) United States
(12) Patent Application Publication
(10) Pub. No.: US 2022/0395489 A1
(43) Pub. Date: Dec. 15, 2022

(54) THERAPEUTIC APPROACH FOR TREATING INFLAMMATORY BOWEL DISEASE

(71) Applicant: THE REGENTS OF THE UNIVERSITY OF CALIFORNIA, Oakland, CA (US)

(72) Inventors: David J. Gonzalez, La Jolla, CA (US); Parambir Dulai, La Jolla, CA (US); Robert Mills, La Jolla, CA (US)

(21) Appl. No.: 17/771,737

(22) PCT Filed: Oct. 28, 2020
(86) PCT No.: PCT/US20/57784

§ 371 Date: Apr. 25, 2022

Related U.S. Application Data
(60) Provisional application No. 62/927,621, filed on Oct. 29, 2019.
(60) Provisional application No. 62/971,148, filed on Feb. 6, 2020.

(51) Int. Cl. A61K 31/4045; A61P 1/04; A61K 31/145; A61K 31/336; A61K 38/08; A61K 45/06; A61K 31/52; G01N 33/68

(52) U.S. Cl. CPC A61K 31/4045; A61P 1/04; A61K 31/145; A61K 31/336; A61K 38/08; A61K 45/06; A61K 31/52; G01N 33/6893; G01N 2800/52; G01N 2800/065

(57) ABSTRACT

Provided herein are compositions and methods to that target microbial proteases to ameliorate the intestinal barrier dysfunction and restore mucosal integrity. They are useful to treat and prevent diseases and disorders caused by pathogenic bacteria in the gastrointestinal system of a subject.

### CROSS-REFERENCE TO RELATED APPLICATIONS

This application claims priority under 35 U.S.C. § 119(e) of U.S. Provisional Application Nos. 62/927,621 and 62/971,148, filed Oct. 29, 2019 and Feb. 6, 2020, respectively, the contents of each of which is incorporated by reference in its entirety into the present application.

### BACKGROUND

Inflammatory bowel disease (IBD) is a term for two conditions (Crohn's disease (“CD”) and ulcerative colitis (“UC”) that are characterized by chronic inflammation of the gastrointestinal (GI) tract. Prolonged inflammation results in damage to the GI tract. Crohn's disease can affect any part of the GI tract (from the mouth to the anus). It often affects the portion of the small intestine before the large intestine/colon. Inflammation in CD can reach through the multiple layers of the GI tract. Damaged areas appear in patches next to areas of healthy tissue. UC occurs in the large intestine (colon) and the rectum. Damaged areas are continuous (not patchy), usually starting at the rectum and spreading further into the colon. Inflammation is present only in the innermost layer of the colon.

Ulcerative Colitis and Crohn's disease are characterized by chronic inflammation of the colon, with severity of mucosal inflammation being associated with a higher risk of work disability, hospitalization, colorectal cancer, and colectomy¹. Non-specific immunosuppressive agents targeting the host, such as steroids, thiopurines, and/or biologics, are used to offset the natural history of disease in patients with moderate-severe inflammation. These therapies are, however, associated with significant risks and often ineffective in adequately managing disease³. Thus, a need exists in the art to provide safe and effective therapies for the treatment of inflammation of the colon and more specifically, ulcerative colitis and Crohn's disease. This disclosure provides formulations that address this unmet need and provides related advantages as well.

### SUMMARY OF THE DISCLOSURE

This disclosure provides a composition comprising, or consisting essentially of, or consisting of one or more protease inhibitors, selected to target a protease expressed by an organism (e.g., a pathogenic organism) related to IBD, UC, CD or a related disease or disorder, alone or in combination with a carrier, e.g., a pharmaceutically acceptable carrier or a biocompatible scaffold. In one aspect, the composition further comprises, or consists essentially of, or consists of, a stabilizer, preservative or agent that provides for enhanced stability for freezing and thawing, or formulation. In one aspect, the protease inhibitors are optionally combined with one or more of other embodiment(s) and/or aspect(s) as disclosed herein. In some embodiments, the composition is for use in a method as disclosed herein.

The proteases in the composition are expressed by one or more of the organisms as identified herein, such as those in FIGS. 5a to 5f, 6b, 6d 7a, 7b, 7d, 8a-8e, 9a, 9b, 14a, and 15m and/or Table 3. In some embodiments, the organism is a Bacteroides organism, e.g., one or more of: Bacteroides vulgatus (also referred to herein as B. vulgatus), Bacteroides dorei (also referred to herein as B. dorei), Bacteroides uniformis (also referred to herein as B. uniformis), Bacteroides ovatus (also referred to herein as B. ovatus), Bacteroides fragilis (also referred to herein as B. fragilis), Bacteroides theta (also referred to herein as B. theta), Bacteroides stercoris (also referred to herein as B. stercoris), Bacteroides cellulosilyticus (also referred to herein as B. cellulosilyticus), Bacteroides xylanisolvens (also referred to herein as B. xylanisolvens), Bacteroides caccae (also referred to herein as B. caccae), or any other Bacteroides species. In some embodiments, the organism is Bacteroides vulgatus and Bacteroides dorei. In some embodiments, the organism is Bacteroides vulgatus. In some embodiments, the organism is Bacteroides dorei. In some embodiments, the organism does not comprise one or more of: Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, or any other Bacteroides species except Bacteroides vulgatus or Bacteroides dorei. In some embodiments, the organism does not comprise Bacteroides theta. Additionally or alternatively, the organism does not comprise Bacteroides fragilis. In some embodiments, the composition further comprises a protease expressed by another organisms, such as those not expressed by the pathogenic organism(s) but expressed by a subject who does or does not comprise the pathogenic organism(s), and/or those expressed by an organisms as disclosed in FIG. 15m. In other embodiments, the composition does not comprise either or both of: a protease not expressed by the pathogenic organism(s) but expressed by a subject who does or does not comprise the pathogenic organism(s), and/or those expressed by an organisms as disclosed in FIG. 15m.

In another aspect, one or more composition(s) as disclosed herein are combined with one or more of other embodiment(s) and/or aspect(s) as disclosed herein. These proteases are identified herein, such as one or more of those in FIGS. 7e, 11e, 11f, 11g, and/or 18a-18e and/or Table 4. In further embodiments, the protease is selected from those disclosed more than once in these Figures and Table of this disclosure.

In some embodiments, the protease is a Bacteroides bacterium protease. Additionally or alternatively, the protease is selected from one or more of a serine protease, a metalloproteinase, an aspartyl protease and a cysteine-protease. In some embodiments, the protease is selected from one or both of a serine protease and/or a cysteine protease.

Also provided herein is a method for one or more of the following: supporting anti-bacterial immunity, correcting dysbiosis, enhancing or supporting the gastrointestinal barrier, supporting or enhancing gastrointestinal motility, localized release of antibiotic compositions, or antagonizing disease-related bacterial infections; treating one or more of: inflammatory bowel disease (IBD), irritable bowel syndrome (IBS), colorectal cancer, chronic inflammation of the colon, colectomy, dysbiosis, colitis, ulcerative colitis (UC), Crohn's disease (CD), enteric infectious disease, diarrheal illness, vaginosis, wound, burns, psoriasis, dermatitis, tooth decay, periodontitis, sinusitis, infection-induced colitis, traveler's diarrhea, psychological stress, psychological disorders, or any of chronic or recurrent disease that is caused by pathogenic bacteria displacing healthy bacteria in the gut or digestive tract; or preventing one or more of: inflammatory bowel disease (IBD), irritable bowel syndrome (IBS), colorectal cancer, chronic inflammation of the colon, colectomy, dysbiosis, colitis, ulcerative colitis (UC), Crohn's disease (CD), enteric infectious disease, diarrheal illness, vaginosis, wound, burns, psoriasis, dermatitis, tooth decay, periodontitis, sinusitis, infection-induced colitis, traveler's diarrhea, psychological stress, psychological disorders, or any of chronic or recurrent disease that is caused by pathogenic bacteria displacing healthy bacteria in the gut or digestive tract. The method comprises, or consists essentially of, or yet further consists of administering to a subject in need thereof, e.g., an effective amount of, a protease inhibitor. In some embodiments, the inhibitor targets a protease expressed by a pathogenic bacterium. Additionally or alternatively, the pathogenic bacteria comprises, or consists essentially of, or yet further consists of a Bacteroides bacterium (such as one or more of: Bacteroides vulgatus, Bacteroides dorei, Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, or any other Bacteroides species). In some embodiments, the protease is selected from one or both of a serine protease or a cysteine protease. In some embodiments, the method further comprises administering to the subject, e.g., an effective amount of, a non-specific immunosuppressive agent optionally selected from a seroid or a thiopurine.

In any aspect(s) and/or embodiment(s) as disclosed herein, the subject for treatment has (such as comprises, shows, expresses, and/or is detected with) one or more of: a high level of a protease as disclosed herein, such as one or more of those expressed by the pathogenic bacteria; a high activity of a protease as disclosed herein, such as one or more of those expressed by the pathogenic bacteria; a high level of a peptide and/or a protease target or a fragment thereof an altered expression of one or more of protein(s) as identified in Table 7, optionally in a sample and further optionally in exosomes of a sample, such as a fecal sample and/or a fecal exosome sample; an altered expression of a tight junction protein; an increased permeability of an epithelial cell layer; a decrease in epithelial cell circularity; or resistance to a conventional treatment. In some embodiments, the conventional treatment is selected from a non-specific immunosuppressive agent. In some embodiments, the peptide is selected from one or more of a dipeptide or an oligopeptide. Additionally or alternatively, the peptide is selected from one or more of: a target of the protease or a fragment thereof. In some embodiments, the target is selected from one or more of a collagen, a mucin or a peptide as identified herein. In some embodiments, the subject is an animal or mammal. In a further embodiment, the mammal is a human patient. In some embodiments, one or more of the level, activity, expression, permeability or circularity is detected and/or quantified in a sample, optionally selected from one or more of: a serum sample, a fecal sample or a GI biopsy sample.

In any aspect(s) and/or embodiment(s) as disclosed herein, the protease inhibitor is administered locally or systemically, such as orally, to the subject.

In any aspect(s) and/or embodiment(s) as disclosed herein, the method can further comprises assaying a sample isolated from the subject for one or more of the following: a level of a protease as disclosed herein, such as one or more of those expressed by the pathogenic bacteria; a protease activity; a level of a peptide and/or a protease target or a fragment thereof; expression of one or more of protein(s) as identified in Table 7, optionally in a sample and further optionally in exosomes of a sample, such as a fecal sample and/or a fecal exosome sample; expression of a tight junction protein; epithelial cell circularity; or permeability of an epithelial cell layer. In some embodiments, the sample is selected from one or more of: a serum sample, a fecal sample or a GI biopsy sample. In one aspect, the sample comprises a fecal sample. In some embodiments, the peptide is selected from one or more of a dipeptide or an oligopeptide. Additionally or alternatively, the peptide is selected from one or more of: a target of the protease or a fragment thereof. In some embodiments, the target is selected from one or more of a collagen, a mucin or a peptide as identified herein. The assaying can occur before, after, or during treatment. The assay allows for personalized therapy and/or to monitor treatment and adjust the composition or therapy as needed.

In another aspect, provided herein is a method to identify a subject suitable for a protease therapy, such as those as disclosed herein. The method comprises, or consists essentially of, or yet further consists of assaying a sample isolated from the subject for one or more of the following: a level of a protease as disclosed herein, such as one or more of those expressed by the pathogenic bacteria; a protease activity; a level of a peptide and/or a protease target or a fragment thereof; expression of one or more of protein(s) as identified in Table 7, optionally in a sample and further optionally in exosomes of a sample, such as a fecal sample and/or a fecal exosome sample; expression of a tight junction protein; epithelial cell circularity; or permeability of an epithelial cell layer. In some embodiments, the sample is selected from one or more of: a serum sample, a fecal sample or a GI biopsy sample. In one aspect, the sample comprises a fecal sample. In some embodiments, the peptide is selected from one or more of a dipeptide or an oligopeptide. Additionally or alternatively, the peptide is selected from one or more of: a target of the protease or a fragment thereof. In some embodiments, the target is selected from one or more of a collagen, a mucin or a peptide as identified herein. In some embodiments, one or more of the following identifies the patient suitable for the protease therapy: higher than normal levels of any one or more of the protease(s), the protease activity, or the peptide(s); an altered expression of one or more of protein(s) as identified in Table 7, optionally in a sample and further optionally in exosomes of a sample, such as a fecal sample and/or a fecal exosome sample; an altered expression of a tight junction protein; a decrease in epithelial cell circularity; or an increased permeability of an epithelial cell layer. In some embodiments, the method further comprises administering to the subject, for example an effective amount of, a protease inhibitor to the identified subject. In some embodiments, the method further comprises administering to the subject, for example, an effective amount of, a conventional treatment agent, such as a non-specific immunosuppressive agent, to the subject.

Further provided is a kit. In some embodiments, the kit comprises, or consists essentially of, or yet further consists of one or more of a protease inhibitor (such as an effective amount of a protease inhibitor). In some embodiments, the inhibitor targets a protease expressed by a pathogenic bacterium. In some embodiments, the kit comprises or consists essentially of, or yet further consists of one or more of reagent(s) and/or buffer(s) for detecting expression and/or activity level(s) of a protease as disclosed herein, expression level of a peptide as disclosed herein, expression (such as level and/or pattern) of a tight junction protein, epithelial cell circularity, and/or permeability of an epithelial cell layer. Additionally or alternatively, the kit is for use in a method as disclosed herein. In some embodiments, the method further comprises a conventional treatment agent, e.g., a non-specific immunosuppressive agent (such as an effective amount of the non-specific immunosuppressive agent(s)). Additionally or alternatively, the method further comprises instructions for use.

### BRIEF DESCRIPTION OF THE DRAWINGS

FIG. 1 summarizes Applicant's study design and database generation. Paired fecal and serum samples were collected from 40 patients with varying severity of Ulcerative Colitis. Samples were processed for proteomics using a Tandem Mass Tag multiplexing workflow. Fecal samples were also subjected to both 16S for microbial composition and shotgun metagenomic analysis for gene profiling and quantification. In parallel, a metabolomics workflow was performed on fecal samples and collected MS2 spectra were analyzed for both metabolites and peptides in two separate computational pipelines. A custom database was compiled from the metagenome of fecal samples to mediate a comparative analysis between shotgun metagenomic and metaproteomic data sets. This eliminated database dependent bias and the shared reference was used for estimating copy number. The shared metagenomic-metaproteomic database Applicant generated for this study consisted of 3,029,818 open reading frames (ORFs), with 2,366,026 having relative DNA abundance, and 46,398 having protein quantification. In total, 133,786 unique peptides were found in the metaproteome analysis. The 16S analysis resulted in 1,986 amplicon sequence variants and the metabolomics analysis consisted of 2,018 MS2 features of which 202 had putative identifications. Serum proteomics resulted in 1,005 proteins quantified. Short peptides were predicted through de novo sequencing and 558 were identified with high confidence (average local confidence >85%). The two exemplified nucleic acid sequences in the right middle box are provided herein as SEQ ID NOs: 1 and 2.

FIGS. 2a to 2c show that multiplexing approach improves the depth and sparsity of metaproteomics data. Fecal proteome samples from UC patients collected for the IBD multiomics database (n=102) were downloaded and reanalyzed using a two-step database approach¹ under a generalized human gut metagenome database². Data from the current study was also re-searched using this database methodology for direct comparisons between datasets. FIG. 2a shows that multiplexed metaproteomic methods increase the total number of proteins quantified. Shown is a bar graph showing the total number of proteins identified when using identical database methodology between the 102 UC samples from the IBD multiomic's database, the 40 UC samples from cohort 1 of this study, and the 205 samples from cohort 2 of this study. FIG. 2b shows that multiplexed metaproteomic methods improve the number of proteins quantified per sample. Displayed are boxplots summarizing the distributions of per-sample protein identifications comparing the same samples from (FIG. 2a). The mean and standard deviation of the distributions are displayed. FIG. 2c shows that multiplexed metaproteomic methods decrease the sparsity of metaproteomic studies. The percentage of missing quantification values for proteins in each data set is shown.

FIGS. 3a to 3h show multi-omic analysis of IBD disease activity. FIG. 3a provides a heatmap of the correlation between clinical metadata values. Hierarchical clustering was performed on spearman correlation values between clinical metrics of patients identifying groups of closely related clinical measurements. FIG. 3b shows that alpha-diversity decreases with active IBD. Pielou evenness based on 16S is plotted for each patient and 95% confidence interval best fit lines are plotted. An R² value is indicated based on the disease activity, diagnosis and their interaction. FIG. 3c shows that beta-diversity correlates with active IBD. Each collected meta-omic dataset is displayed by a principle coordinate analysis showing the first two axis. Each sample is colored by the disease activity state and has a shape corresponding to diagnosis. Adonis R² values are shown to demonstrate the effect size of disease activity when accounting for disease activity, diagnosis and their interaction. The distance metrics used are weighted unifrac for each dataset other than proteomic datasets which use the Bray-Curtis distance metric, and the UC cohort 1 which shows unweighted unifrac. FIG. 3d shows 16S phyla composition by disease activity states. The average phyla compositions of groups of patient samples are shown in bar plots. FIG. 3e shows data type correlations. Pearson correlations between data types are displayed in a heat map. The Bray-Curtis distance metric was used for all data types and correlations were performed on distance matrices through Mantel's test. FIG. 3f shows predicting severity of disease activity. The mean squared error from 100 iterations of random forest analyses trained to predict the partial Mayo disease activity are displayed in boxplots ordered from the strongest predictive capability (metabolome) to the least predictive capability (serum proteome). FIG. 3g shows metaproteome composition by disease activity states. The relative abundances of human and microbial proteins were averaged by disease activity states and plotted by different patient categories. FIG. 3h provides that top metabolite classes correlated with UC disease activity. Metabolite abundances by ClassyFire direct parent annotations were averaged and linear regressions were performed on disease activity. The r-values of the top 10 positively and negatively correlated classes of chemicals are plotted by diagnosis and cohort.

FIGS. 4a to 4g evaluates alpha and beta-diversity relationships to disease activity and data type comparisons. In addition to Pielou's evenness metric for alpha-diversity reported in FIG. 3b, other alpha-diversity metrics were tested for their relationship to disease activity in 16S data. These metrics included Shannon's index (FIG. 4a), Observed OTUs (FIG. 4b), and Faith's PD (FIG. 4c). R² statistics are reported from an ordinary least-squares regression using the formula (Disease Activity+Diagnosis+Disease Activity:Diagnosis). FIG. 4d provides PCoA of shotgun metagenomic data from UC cohort 1. Bray-Curtis distance metric was used and the first two principal coordinates are displayed. Samples are sized by fecal calprotectin abundance and colored by the associated partial Mayo severity scores. FIG. 4de provides PCoA of 16S data using the Bray-Curtis distance metric. Samples were sized and colored as described for (FIG. 4d). FIG. 4f provides PCoA of fecal metaproteomics data after host proteins were removed using the Bray-Curtis distance metric. Samples were sized and colored as described for (FIG. 4a). FIG. 4g provides data type correlations. Pearson correlations between data types are displayed in a heat map. The Bray-Curtis distance metric was used for all data types other than the metagenome and 16S, which used unweighted UniFrac. Correlations were performed on distance matrices from cohort 1 through Mantel test.

FIGS. 5a to 5f show taxonomic composition plots by data type. Phyla composition of the 16S (FIG. 5a), MG (FIG. 5b), and MP (FIG. 5c) data are shown for each fecal sample analyzed. Samples are ordered by the relative disease activity the patient is currently experiencing. Patients are split by independently processed cohort for UC patients and diagnosis. The top 7 most abundant phyla are displayed with all other phyla grouped in “Others”. Genera composition plots for each patient is also shown (FIGS. 5b-5f). For genera plots, the top 10 genera are displayed with all other phyla grouped in “Others”.

FIGS. 6a to 6e show that uneven CD fecal samples are dominated by Enterobacteriaceae via characterizing uneven samples. FIG. 6a provides alpha diversity (using Pielou's evenness metric) by disease activity as shown in FIG. 3b, but highlighting classification of samples as uneven when below Pielou Evenness of 0.5. FIG. 6b provides that 16S beta-diversity is strongly influenced by community evenness. The weighted UniFrac distance metric was used and each sample was classified by community evenness, diagnosis and whether the most abundant 16S feature was from the family Enterobacteriaceae. FIG. 6c characterizes the most abundant 16S features. Each sample was classified as either “Uneven” (Pielou Evenness<0.5) or “Other” as shown in (FIG. 6a). Abundances of each amplicon sequence variant were summed by their highest resolution taxonomic annotation and the most abundant feature of samples are represented in a donut plot. The inside ring represents the fractional composition of each patient subgroup and the outside rings represents the number of patients within each subgroup whom share a similar most abundant feature. Less common features for each patient subgroup are counted as “Other”. FIGS. 6d-6e further show that evenness correlates with changes in bile acids. FIG. 6d provides random forest prediction of evenness through 16S. A scatterplot depicting the accuracy of the developed model on the testing subset of the samples is shown, the x-axis plots the true value, and the y-axis plots the predicted value. The annotation information available, importance values, and importance rank of the top 10 sequences are shown to the right. FIG. 6e shows that evenness correlates with changes in bile acid abundances. Best-fit lines with 95% confidence intervals are shown for three bile acids according to the 16S based Pielou evenness for each sample (x-axis) and the MS abundance (y-axis). 12-Ketodeoxycholic acid is displayed as the straight line on top at the evenness of 0.7, Lithocholic acid is displayed as the straight line in the middle at the evenness of 0.7, and Cholic acid is displayed as the straight line at the bottom at the evenness of 0.7.

FIGS. 7a to 7f show that integrated metagenomic-metaproteomic analyses reveal Bacteroides proteases distinguishing a subset of active UC patients. FIG. 7a shows taxonomic biases among proteins correlated to disease activity. Linear regressions against disease activity were performed for each protein quantified and the taxonomic origins of all highly associated (r>0.3 or r<−0.3) are plotted per patient cohort. FIG. 7b provides comparison of biases in the taxonomic origins of highly associated microbial open-reading frames at the MG or MP level. Linear regressions were performed as in (FIG. 7a), and the percent representation of taxa in positive correlations (r>0.3) and negative correlations (r<−0.3) are plotted by Log 10 transformation. FIG. 7c shows functional shifts in Bacteroides during active IBD. In the top panel, the Bacteroides proteins associated with disease activity (r>0.3) from (FIG. 7a) were compared to remaining identified Bacteroides proteins to identify putative functional shifts related to UC disease activity. The bottom panel provides KEGG composition of Bacteroides proteins. FIG. 7d provides species-level investigation of Bacteroides in MG of UC patients and shows the most abundant species in UC patients to be B. vulgatus and B. dorei. Bacteroides species composition plots are shown for categories of UC disease activity, as well as the average within each cohort. Above each composition plot are dot plots indicating the average abundance of Bacteroides reads in the MG, or a violin plot of the general distribution in the UC cohort. FIG. 7e provides that correlation of Bacteroides proteases and enzymes to UC disease activity and shows the strongest correlations between UC disease activity and serine proteases from abundant Bacteroides species such as B. vulgatus and B. dorei. The species level annotation of enzymes identified in different Bacteroides species was compared in a heatmap showing the correlations of each enzyme per species. FIG. 7f shows that patients with Bacteroides protease overproduction correlates with increased disease activity. An outlier approach comparing B. vulgatus and B. dorei metagenomic abundance to the summed protein abundances from B. vulgatus and B. dorei proteases was taken to identify groups of UC patients with higher or lower than metagenomically expected protease presence. A bagplot is shown with a best-fit line and over or under-producer status was determined by outlier status above or below the best-fit line. The disease activity of overproducers, underproducers, and other patients are individually plotted over boxplots. T-test p-values are displayed above the boxplots.

FIGS. 8a to 8e provide comparison of genera annotations from genes and proteins correlated to disease severity. The genus composition of genes and proteins correlated to disease activity were compared with different levels of sparsity as a requirement for being deemed “correlated”. Stacked bar charts summarize the number of genes or proteins from the 10 most common genus assignments when correlated to either partial Mayo severity in UC cohorts or CDAI in CD patients. Only genes or proteins with |r|>0.3 from linear regression were included. FIG. 8a provides genus composition of significant positively and negatively correlated genes from the MG with no sparsity requirement. FIG. 8b provides genus composition of significantly positively and negatively correlated proteins from the MP with no sparsity requirement. FIG. 8c provides genus composition of associated proteins as in FIG. 7a, but without removing host proteins (genus Homo). FIG. 8d provides that genes correlated to disease activity from the MG when filtering out genes appearing in less than 40% of patients within each category. FIG. 8e provides a summary of comparing the portions of positively and negatively correlated genes and proteins from each patient cohort when examining the top 10 genera identified in the MG. This analysis is analogous to FIG. 7b, but displaying the top MG genera.

FIGS. 9a to 9c provide comparison of genera and functional annotations from genes and proteins correlated to disease severity in CD subtypes. FIG. 9a provides genus level barcharts of significantly correlated genes or proteins stratified by CD subtype. The genus composition of genes and proteins from either the MG or MP were correlated to CDAI and shown in stacked bar charts. Only genes or proteins with |r|>0.3 from linear regression were included, and the top 10 genera are displayed with other genera compiled into an “Others” category. FIG. 9b provides CD subtypes genus level association comparison. The portion of genes or proteins correlated with disease activity from (FIG. 9a) are plotted by a Log 10 comparison between the proportions of positive to negative correlations. Genes correlated to disease activity from the MG when filtering out genes appearing in less than 40% of patients within each category. FIG. 9c provides CD subtypes functional association comparison. This analysis is analogous to (FIG. 9b) but summarizing the associations to KEGG functional category annotations in the MP.

FIG. 10 shows that patients with overproduction of Bacteroides vulgatus proteases have increased endoscopic disease activity. The disease activity of overproducers, underproducers, and other patients are individually plotted over boxplots. T-test p-values are displayed above the boxplots.

FIGS. 11a to 11l provides assessing proteolysis in UC patients and Bacteroides supernatant. FIG. 11a shows abundances of dipeptides increases with disease activity. The average relative abundance of metabolomic features annotated as dipeptides is plotted according to disease activity with 95% confidence intervals shown per group of patients. FIG. 11b shows that peptide fragments are more abundant during active UC. The number of peptides identified through a de-novo peptidomic workflow is plotted alongside UC disease activity. Significance and Pearson correlation of the linear relationship is shown with 95% confidence intervals for UC cohort 1. FIG. 11c provides that the number of peptide fragments from human proteins indicates potential targets of UC proteolysis. The gene symbol for the human proteins with the most short peptides present are shown on the y-axis and the quantity of peptides is shown on a log 10 transformed x-axis. The proteins are colored by common categories of the observed proteins. Proteins fitting into two categories have both colors represented. FIG. 11d shows class of protease activity in B. vulgatus supernatant. Eight-fold concentrated supernatant from overnight culture of B. vulgatus was subjected to the EnzChek protease activity assay (Invitrogen) in the presence of different classes of protease inhibitors, as illustrated in the right panel. Vehicle controls were used to determine the percent inhibition from each inhibitor and the mean and SEM from 3 independent experiments are displayed. Protease inhibitors included 10 mM AEBSF (Serine), 100 μM E-64 (Cysteine), 2.5 mM GM6001 (Metallo) and 180 μM Pepstatin A (Aspartyl). FIG. 11e provides molecular function composition of enzymes and proteases in Bacteroides supernatant. Supernatant from overnight cultures of B. dorei, B. theta, and B. vulgatus, were analyzed by LC-MS³ based proteomics. The average composition of GO Molecular Function for any protein annotated as an enzyme, peptidase or protease is displayed by species. FIG. 11f shows proteases specifically enriched within B. vulgatus supernatant. Proteases abundant within B. vulgatus supernatant were ranked and plotted by the relative abundance difference of each protease within B. vulgatus supernatant in comparison to B. theta supernatant. Comparisons between B. dorei and B. theta are also shown. FIG. 11g provides ranking of B. vulgatus proteases by summed correlations to UC disease activity. The correlation values (r) between UC disease activity and B. vulgatus and B. dorei proteases were summed. The sums from the top-10 ranked proteases are shown with the colors of each bar representing protease class. FIG. 11h provides comparison of B. vulgatus proteases identified in UC patients and in supernatant proteomics. Protein names were compared for B. vulgatus or B. dorei proteases correlated to UC activity (r>0.3) in either cohort, with the proteases identified in B. vulgatus or B. dorei supernatant, and the proteases identified in B. theta supernatant. FIGS. 11i to 11l further show that bacterial and host proteolysis correlates to disease severity and fecal to serum ratios of serine protease inhibitors predict severity in patient subpopulations. FIG. 11i shows that SerpinA1 and SerpinA3 fecal to serum ratios correlate to disease severity. Proteome abundances of serine protease inhibitors (SerpinA1 and SerpinA3) were compared by fecal/serum ratios. These ratios are plotted on the y-axis with values of a severity metric on the x-axis (either fecal calprotectin abundance, or partial Mayo severity scores). A best fit and 95% confidence interval is plotted for each plot. Different patient sub populations were plotted including all patients, patients with Mayo endoscopic score=0 and patients with Mayo endoscopic score=0 or 1. Significant associations (p<0.05) are observed in the left column, top one in the middle column and the bottom one of the right column. FIG. 11j shows that peptide fragments are more abundant at high severity. The number of peptides identified is represented on the y-axis and the partial Mayo clinical score is represented on the x-axis. Significance and Pearson correlation of the linear relationship is shown with a 95% CI drawn surrounding the best-fit line. FIG. 11k shows neutrophil proteases are significantly correlated to disease severity. The proteome relative abundance is illustrated on the y-axis with the partial Mayo scoring on the x-axis. *** Indicates a p<0.001 for the linear relationship between each protease. FIG. 11l shows that the metaproteome of patients with mucosal healing have large fluctuations associated with histological remission. A volcano plot depicting the Log 2(fold change) and log 10(p-value) for each protein in the metaproteome. Significance was determined by a |π|⁷⁰>1, and protein groups of interest are highlighted in the legend.

FIGS. 12a to 12f show that dipeptides and peptide fragments are increased in active UC patients and Bacteroides protease enriched patients. FIG. 12a shows that Bacteroides protease overproducers have increased average dipeptide abundance in their fecal samples. FIG. 12b shows significant cohort differences in the abundance of peptide identifications resulted in limited analysis of de novo peptides from UC cohort 2. Overproducers from cohort 1 had increased peptide fragments in comparison to the general UC cohort 1. FIG. 12c shows that peptide termini indicate unique proteolysis of human and microbial proteins. The frequency of each amino acid within the N and C terminus of human and de-novo peptides was compared to either the human proteome or the total amino acid content of de novo peptides. The Y-axis represents the percent difference of each residue and the letter indicates the amino acid associated with the difference. The N and C terminus are shown separately and each residue is colored by chemical property (Green=polar, Black=Hydrophobic, Red=Acidic, Blue=Basic, Purple=Neutral). FIG. 12d shows that highly active UC patients from cohort 1 had increased identification of peptide fragments by de novo sequencing of metabolomics data from fecal samples. FIG. 12e shows cohort differences in peptide identification abundance resulted in limited resolution between active and inactive UC patients, despite similar trends identified in cohort 1 and cohort 2. FIG. 12f shows that dipeptide and amino acids correlated with severity. Metabolite identifications for dipeptides and amino acids with a significant (p<0.05) association with partial Mayo severity are shown. The abundance of each peptide is plotted on the y-axis and the partial Mayo severity score is plotted on the x-axis. In addition, 95% confidence intervals are plotted around a best-fit line.

FIGS. 13a to 13c show that host protein networks highlight that host enzyme activity and regulation is associated with UC severity. FIG. 13a show that enzyme inhibition is enriched within serum proteins related to disease activity. A network is displayed showing connections of serum proteins correlated to partial Mayo disease activity (|r|>0.2), which were determined through String-DB. Edges are sized by combined confidence in the interaction, with only high confidence connections displayed (>0.7; theoretical max confidence=1). Nodes are sized and colored depending on the correlation to the partial Mayo disease activity score. A functional enrichment for enzyme inhibition activity was found and proteins annotated with this function are indicated by a diamond shape and a black border. The connecting edges of enzyme inhibitor proteins are colored dark gray while all other connections are colored light gray. FIG. 13b shows serum proteins associated to Calprotectin. Pearson correlations were performed comparing fecal calprotectin relative abundances to serum proteome relative abundances. The top 10 positively and negatively correlated serum proteins are plotted by their Pearson correlation coefficient (r). Positively associated proteins are shown in red while negatively associated proteins are shown in blue. FIG. 13c provides that peptidase related proteins are highly connected within fecal exosome proteins correlated to disease activity. Exosome proteins were highly enriched among the human fecal proteins correlated to Ulcerative Colitis disease activity. Displayed is a network of exosome proteins highly correlated with the partial Mayo severity score (r>0.5). Edges are sized by combined confidence in the interaction, with only high confidence connections displayed (>0.7; theoretical max confidence=1). Nodes are sized and colored depending on the correlation to the partial Mayo disease activity score. A functional enrichment for peptidase activity and peptidase regulator activity was found and a thickened border indicates proteins annotated with these functions. Edges connecting to proteins with peptidase related activity are colored dark gray while all other connections are colored light gray.

FIGS. 14a to 14b determines Bacteroides species effect in co-culture with Caco-2 cells, and protease inhibitor specificity. FIG. 14a shows that Bacteroides vulgatus and Bacteroides dorei, but not other Bacteroides species significantly decrease TEER after 38 hours co-culturing. Barplots showing the mean and standard deviation from 3 technical replicates of Caco-2 co-culturing with the 6 most abundant species identified in the metagenome of patients with UC. FIG. 14b provides growth curves of Bacteroides vulgatus with protease inhibitors under different growth conditions. OD600 was measured at indicated time points and a non-linear fit is shown from technical triplicates of each condition. Growth was measured under anaerobic conditions in BHI-S media and under aerobic conditions +5% CO₂ in DMEM.

FIGS. 15a to 15n show that protease inhibition protects from Bacteroides vulgatus and fecal transplant induced pathology in vitro and in vivo. FIG. 15a provides a schematic describing the in vitro studies using Caco-2 cell monolayers and Bacteroides spp. FIG. 15b provides that protease inhibitor cocktail significantly reduces the Caco-2 resistance reduction when co-culturing Bacteroides vulgatus and shows a significant improvement in epithelial barrier resistance when applying a protease inhibitor cocktail to epithelial cells cultured with B. vulgatus after 22 and 38 hours of incubation. Caco-2 cells were grown in monolayers on transwell for 2.5 weeks before inoculating Bacteroides vulgatus or Bacteroides thetaiotamicron at a multiplicity of infection (MOI) of ˜5. Transepithelial electrical resistance (TEER) was measured at the given hours post inoculation. A timeseries is plotted with the standard error of the mean (SEM) from 3 biological replicates containing 3 technical replicates within each experiment. FIG. 15c shows that protease inhibitor cocktail does not significantly influence the number of colony forming units during Caco-2 co-culturing with Bacteroides vulgatus. Colony forming units from above the transwell insert were estimated through serial dilution and plating onto BHI-S plates under anaerobic conditions. Plotted are the mean CFUs from each experimental condition from three biological replicates containing 3 technical replicates per experiment. FIG. 15d provides representative images from confocal microscopy of the transwell experiments. Following 38 hours of co-culturing, the Caco-2 transwell inserts were fixed and stained for immunofluorescence of tight junction proteins, Zo-1 and Occludin. A representative image from untreated Caco-2 cells, Caco-2 cells co-cultured with Bacteroides vulgatus, and Caco-2 cells co-cultured with Bacteroides vulgatus and a protease inhibitor cocktail are shown. FIG. 15e provides quantification of cell circularity in the images from FIG. 15d. FIG. 15f shows experimental design of monocolonized IL10−/− mouse study. Mice were inoculated with B. vulgatus. During 10-weeks of colonization, a protease inhibitor cocktail was continuously administered through the drinking water of one cage of B. vulgatus mice. FIG. 15g provides representative images from histological analysis of the colonic epithelium of monocolonized mice. FIG. 15h provides colitis scores from histological assessment of monocolonized mice. FIG. 15i provides crypt length of monocolonized mice. FIG. 15j shows experimental design of humanized IL10−/− mouse study. Fecal samples from three patients with abundant Bacteroides proteases and three patients without abundant Bacteroides proteases were transplanted into 6× gnotobiotic mice per patient sample. During 8-weeks of colonization, a protease inhibitor cocktail was continuously administered through the drinking water of 3 mice per patient sample. Mice were sacrificed after 8-weeks and macroscopic organ measurements were taken. FIGS. 15k-15l provide barplots showing the mean and standard error of the mean are shown for colon length (FIG. 15k), and spleen weight (FIG. 15l). FIG. 15m provides species representation of proteases in the fecal metaproteome of humanized mice. The fecal samples from one group of humanized mice with abundant or low Bacteroides proteases was subjected to LC-MS³ based metaproteomics. It shows a higher abundance of Bacteroides vulgatus proteases within the fecal samples of mice transplanted with patient sample H19 than in fecal samples from patient sample L3. The relative abundance from identified proteases is shown based on the species annotation of each protease. FIG. 15n provides cumulative protease comparisons. A venndiagram is shown comparing the protein names of B. vulgatus or B. dorei proteases from four proteomics experiments represented in this study; the significant findings from two UC cohorts, the proteases identified uniquely in the supernatant of B. vulgatus or B. dorei, and the proteases confirmed to be increased within the humanized mice experiments. A full list of the Bacteroides proteases identified in this study can be found in Table 4.

FIGS. 16a and 16b provide supplementary images from confocal microscopy of Caco-2 monolayer and Bacteroides co-culture experiments. FIG. 16a shows evaluation of specificity and optimum concentration of primary antibodies. Representative images from untreated Caco-2 cells after imaging preparation with only Zo-1 primary antibodies added (far left), only occluding antibodies added (second from left), both primary antibodies at the recommended dilution (second from right), and both primary antibodies diluted 5× from recommended dilution. The antibodies were determined to be specific and the 5× dilution of primary antibodies were used for remaining imaging experiments. FIG. 16b provides individual channels from imaging study displayed in FIG. 11c. Zo-1 is displayed in red in the far left, Occludin is shown second from the left, dapi is shown in the middle, phalloidin is shown second from the right, and the merge of all 4 channels is shown on the far right.

FIGS. 17a to 17v provide macroscopic measurements from germ-free mouse experiments. FIGS. 17a-17h provide barplots showing the mean and standard error of the mean are shown for macroscopic organ measurements from B. vulgatus monocolonized IL10^{-/-} mice with or without administration of a protease inhibitor cocktail in the drinking water over a 10-week colonization. Measurements include total weight (FIG. 17a), colon length (FIG. 17b), ratios of the colon weight to length (FIG. 17c), colon weight (FIG. 17d), caecum weight (FIG. 17e), fat pad weight (FIG. 17f), liver weight (FIG. 17g) and spleen weight (FIG. 17h). FIGS. 17i-17v provide barplots showing the mean and standard error of the mean for macroscopic organ measurements from IL10^{-/-} germ-free mice transplanted with fecal samples from UC patients. Each dot represents the average per cage with each cage having 2-3 mice for a particular donor and condition. Measurements include body weight (FIG. 17v), caecum weight (FIGS. 17i and 17u), fat pad weight (FIGS. 17j and 17s), liver weight (FIGS. 17k and 17r), spleen weight (FIG. 17t), body weight (FIG. 17l), colon weight (FIGS. 17m and 17o), colon length (FIG. 17p) and colon weight to length ratios (FIGS. 17n and 17q). * p-value<0.05, ** p-value<0.01, *** p-value<0.001, **** p-value<0.0001.

FIGS. 18a to 18e provide Bacteroides vulgatus and Bacteroides dorei protease abundances and correlations to peptide fragments. FIG. 18a shows per patient protease abundances in comparison to metagenomic abundance of B. vulgatus and B. dorei. The relative abundance of each protease identified with a taxonomic assignment to B. vulgatus or B. dorei were hierarchically clustered, and plotted in a heatmap. Z-scores calculated between samples are represented in each box with darker red representing a higher abundance within the patient. Plotted above the heatmap are the relative frequency of B. vulgatus and B. dorei reads within the metagenomes of each patient. FIGS. 18b and 18d show validation of Bacteroides proteases in severe UC. FIG. 18c shows bifidobacterium protease in ileocolonic CD. FIG. 18e shows correlation of each B. vulgatus or B. dorei protease to the abundance of high confidence peptide fragments (ALC >85%) identified in the metabolome. The pearson correlation coefficient (r-value) is plotted on the x-axis with each protease plotted on the y-axis. If multiple proteases with identical annotations were detected, they were plotted as individual dots within the same protease.

### DETAILED DESCRIPTION

It is to be understood that this invention is not limited to particular embodiments described, as such can, of course, vary. It is also to be understood that the terminology used herein is for the purpose of describing particular embodiments only, and is not intended to be limiting, since the scope of the present invention will be limited only by the appended claims. Throughout this disclosure, various technical publications are referenced by an Arabic numeral. The complete citations for these publications can be found immediately preceding the claims and are incorporated herein by reference.

Throughout and within this disclosure, reference is made to patent and technical literature, the bibliographic citation for which is provided in the text or by an Arabic numeral, the bibliographic citation for which is found in the Reference section, immediately preceding the claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All technical and patent publications cited herein are incorporated herein by reference in their entirety. Nothing herein is to be construed as an admission that the invention is not entitled to antedate such disclosure by virtue of prior invention.

The practice of the present technology will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration and molecular weight, including ranges, are approximations which are varied (+) or (−) by increments of 1.0 or 0.1, as appropriate, or alternatively by a variation of +/−15%, or alternatively 10%, or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a bacterium” includes a plurality of bacteria, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure. Embodiments defined by each of these transition terms are within the scope of this disclosure.

“Optional” or “optionally” means that the subsequently described circumstance can or cannot occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides, antibodies and proteins that are isolated from other cellular proteins or components and is meant to encompass both purified and recombinant ones. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide, polynucleotide or nucleic acid, intends those having minimal homology while still maintaining desired structure or functionality. Unless specifically recited herein, it is contemplated that any nucleic acid, polynucleotide, polypeptide or protein mentioned herein also includes equivalents thereof. For example, an equivalent intends at least about 70%, or alternatively 80% homology or identity and alternatively, at least about 85%, or alternatively at least about 90%, or alternatively at least about 95%, or alternatively 98% percent homology or identity across the protein or a particular fragment thereof, and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank +EMBL+DDBJ+PDB+GenBank CDS translations+SwissProtein+SPupdate+PIR. Details of these programs can be found at the following Internet address: ncbi.nlm.nih.gov/cgi-bin/BLAST.

“Homology” or “identity” or “similarity” refers to sequence similarity between two peptides or between two nucleic acid molecules. Homology can be determined by comparing a position in each sequence which can be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a peptide and/or protein from one sample can be directly compared to the expression level of that peptide and/or protein from a control or reference sample. In another aspect, the expression level of a peptide and/or protein from one sample can be directly compared to the expression level of that peptide and/or protein from the same sample following an administration as disclosed herein.

As used herein, the term “overexpress” with respect to a cell, a tissue, an organ, a sample, or a subject comprises and/or expresses a protein to an amount that is greater than the amount that is produced in a control cell, a control tissue, a control organ, a control sample, or a control subject. A protein that is overexpressed can be endogenous to the host cell or exogenous to the host cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the term “animal” refers to living multi-cellular vertebrate organisms, a category that includes, for example, mammals and birds. The term “mammal” includes both human and non-human mammals.

A “subject” or “patient” of diagnosis or treatment is a cell or an animal such as a mammal or a human. Non-human animals subject to diagnosis or treatment and are those subject to infections or animal models, for example, simians, murines, such as, rats, mice, chinchilla, canine, such as dogs, leporids, such as rabbits, livestock, sport animals and pets. The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human. In some embodiments, they refer to and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult.

In some embodiments, the subject is at risk of a disease as disclosed herein. In some embodiments, the subject is suspect of having a disease as disclosed herein. In some embodiments, the subject is pre-symptomatic, such as a pre-symptomatic mammal or human. In some embodiments, the subject has minimal clinical symptoms of a disease as disclosed herein. The subject can be a male or a female, adult, an infant or a pediatric subject. In an additional aspect, the subject is an adult. In some instances, the adult is an adult human, e.g., an adult human greater than 18 years of age.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein. A signal generated by a dateable label is referred to herein as a detectable signal.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection can be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. The term also includes sequences conjugated to the polynucleotide that will provide a signal upon expression of the inserted sequences, such as green fluorescent protein (GFP) and the like. The label can be detectable by itself (e.g., radioisotope labels or fluorescent labels) or, in the case of an enzymatic label, can catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label can be simply detected or it can be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response can be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue®, and Texas Red. Other suitable optical dyes are described in the Haugland, Richard P. (1996) Handbook of Fluorescent Probes and Research Chemicals (6th ed.).

As used herein, the terms “treating,” “treatment” and the like are used herein to mean obtaining a desired pharmacologic and/or physiologic effect. The effect can be prophylactic in terms of completely or partially preventing a disorder or sign or symptom thereof and/or can be therapeutic in terms of a partial or complete cure for a disorder and/or adverse effect attributable to the disorder. In one aspect, the term “treatment” excludes prophylaxis. Examples of “treatment” include but are not limited to: preventing a disorder from occurring in a subject that can be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder. In some embodiments, it refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods are intended to be used as a sole therapy or in combination with other appropriate therapies.

In some embodiments, the term “disease” or “disorder” as used herein refers to an inflammation of the gastrointestinal (GI) tract (which are used interchangeably), a status of being diagnosed with such disease, a status of being suspect of having such disease, or a status of at high risk of having such disease. Additionally or alternatively, the term “disease” or “disorder” refers to a disease that is caused by pathogenic bacteria displacing healthy bacteria in the gut or digestive tract. In a further embodiment, the disease is one or both of chronic or recurrent. In one embodiment, the disease is selected from one or more of: lack of or a reduce in anti-bacterial immunity, dysbiosis, abnormal gastrointestinal barrier, reduced or increased gastrointestinal motility, bacterial infections (such as disease-related). Additionally or alternatively, the disease is selected from one or more of: inflammatory bowel disease (IBD), irritable bowel syndrome (IBS), colorectal cancer, chronic inflammation of the colon, colectomy, dysbiosis, colitis, ulcerative colitis (UC), Crohn's disease (CD), enteric infectious disease, diarrheal illness, vaginosis, wound, burns, psoriasis, dermatitis, tooth decay, periodontitis, sinusitis, infection-induced colitis, traveler's diarrhea, psychological stress, psychological disorders, or any disease (such as chronic and/or recurrent) that is caused by pathogenic bacteria displacing healthy bacteria in the gut or digestive tract. In some embodiments, a disease as disclosed herein is resistant to a conventional treatment. In some embodiments, a disease as used herein refers to one or more of those correlated to and/or caused by an increased level (such as expression and/or activity) of a protease, such as those as disclosed herein, for example IBD, UC or CD. In some embodiments, the disease is IBD. In some embodiments, the disease is ulcerative colitis (UC). In some embodiments, the disease is Crohn's disease (CD). In further embodiments, the diseases is one or more of: colonic CD, ileal CD or ileocolonic CD.

To “prevent” intends to prevent a disorder or effect in vitro or in vivo in a system or subject that is predisposed to the disorder or effect.

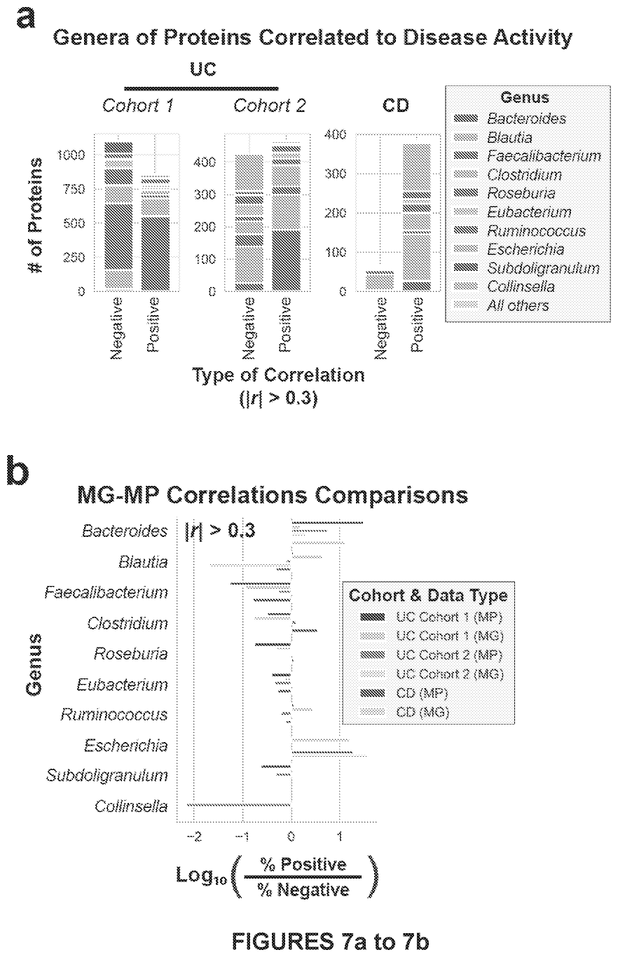

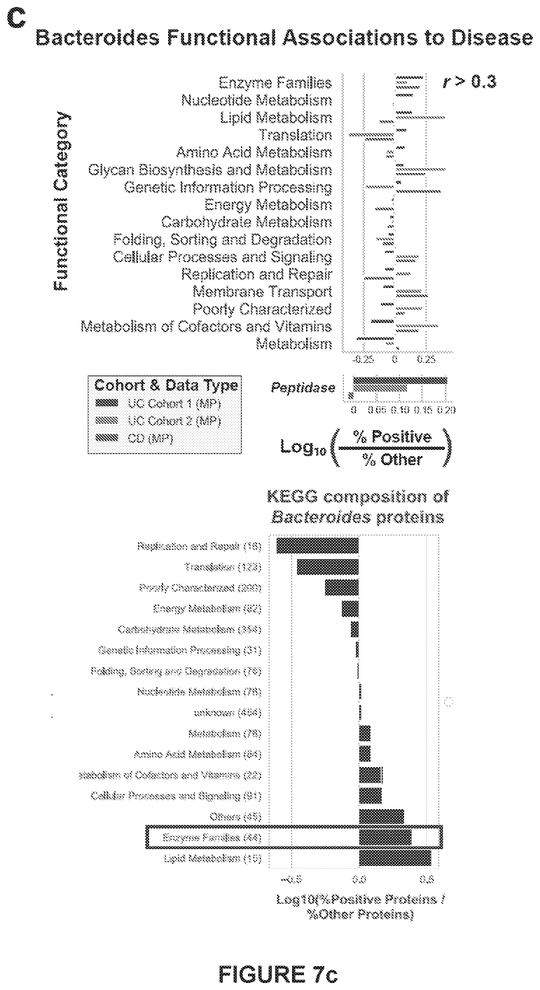

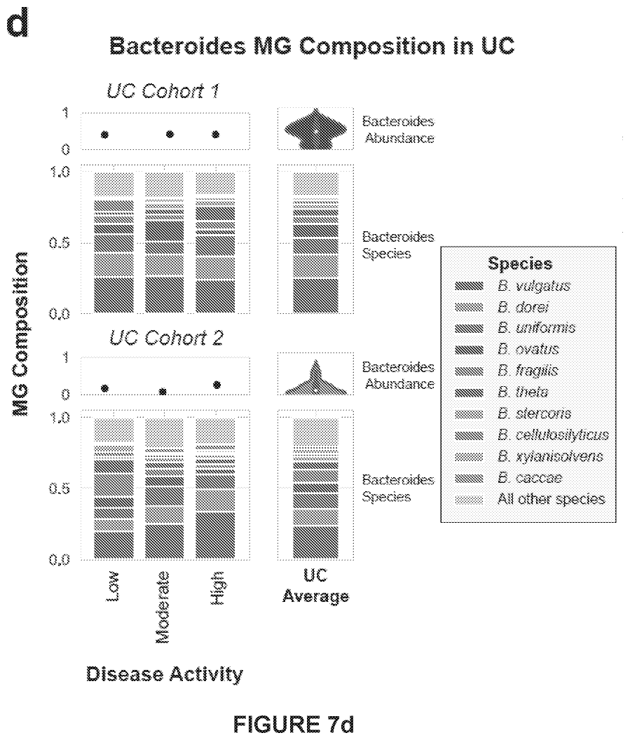

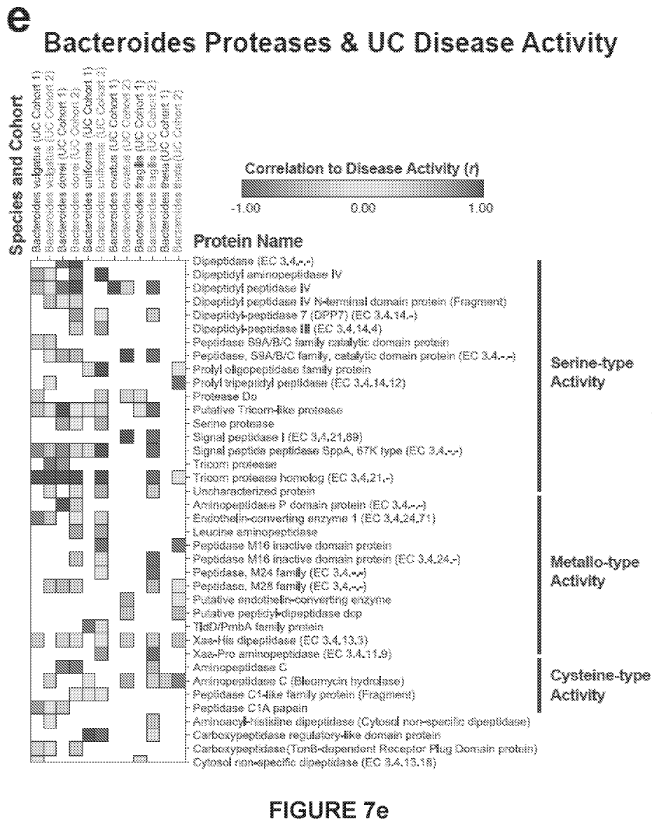

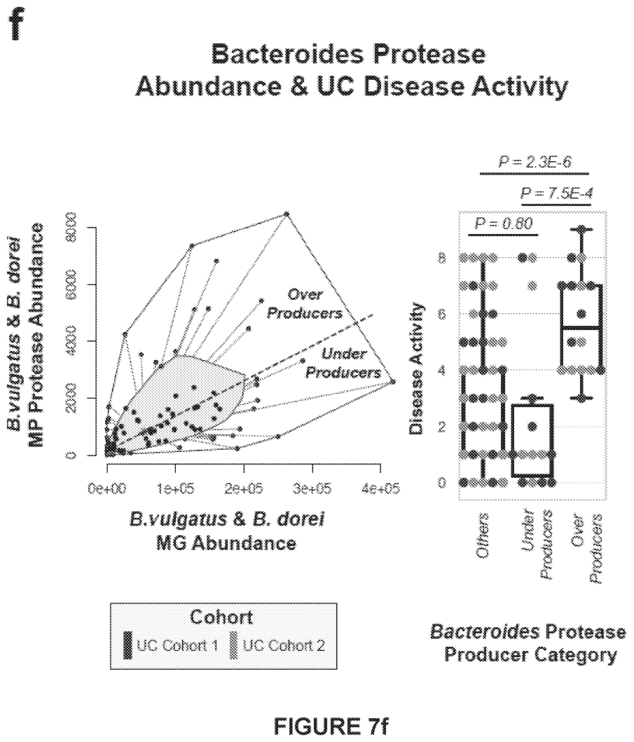

A pathogenic bacterium, which is also referred to as a pathologenic bacterium, is an organism that can perform one or two or all three of the following: disrupting intestinal epithelial permeability, and causing associated inflammatory conditions as well as related disease, for example as a disease as disclosed herein. A non-limiting example of a pathogenic bacterium is Bacteroides. In a further embodiment, the pathogenic bacterium is a bacterial species selected from one or more of: Bacteroides vulgatus, Bacteroides dorei, Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, any other Bacteroides species, and/or a Bacteroides species identified in FIG. 7d and/or Table 3. In some embodiments, the pathogenic bacterium is selected from one or both of Bacteroides vulgatus or Bacteroides dorei. In some embodiments, the pathogenic bacterium is Bacteroides vulgatus. In some embodiments, the pathogenic bacterium does not exist in healthy subjects and/or subjects free of a disease as disclosed herein. In some embodiments, the pathogenic bacterium exists in healthy subjects and/or subjects free of a disease but is pathogenic due to its abnormal amount, such as occupying a larger proportion of the microbiota than in healthy subjects and/or subjects free of a disease.

The term “culturing” refers to the in vitro propagation of cells or organisms on or in media of various kinds. It is understood that the descendants of a cell grown in culture cannot be completely identical (i.e., morphologically, genetically, or phenotypically) to the parent cell. By “expanded” is meant any proliferation or division of cells.

The term “protease” refers to an enzyme that catalyzes (increases the rate of) proteolysis, the breakdown of proteins into smaller polypeptides or single amino acids. In some embodiments, a protease performs its function by cleaving the peptide bonds within proteins by hydrolysis. Proteases can be classified into seven broad groups based on catalytic residue: Serine proteases—using a serine alcohol; Cysteine proteases—using a cysteine thiol; Threonine proteases—using a threonine secondary alcohol; Aspartic proteases—using an aspartate carboxylic acid; Glutamic proteases—using a glutamate carboxylic acid; Metalloproteases—using a metal, such as zinc; and asparagine peptide lyases—using an asparagine to perform an elimination reaction (not requiring water). In some embodiments, the term “protease” as used herein can refer to any one or more protease(s), such as those discussed herein, e.g., in Experimental Examples, Figures, and/or Tables. In some embodiments, the protease is a peptidase, such as a bipeptidase. In some embodiments, the protease is selected from one or more of the following: a serine-type endopeptidase, a serine-type peptidase, a metalloendopeptidase, a hydrolase, a dipeptidyl-peptidase, a serine-type aminopeptidase, an aminopeptidase activity, and/or a metallopeptidase. In some embodiments, the protease is one or more of the following: a serine-protease, a metalloproteinase, an aspartyl protease and a cysteine-protease. In some embodiments, the protease is one or more of the following: a serine-protease and a cysteine-protease. In yet further embodiments, the protease refers to a serine protease.

In further embodiments, the term “protease” as used herein refer to any one or more bacterial protease(s). In yet further embodiments, the term “protease” as used herein refer to any one or more bacteroids protease(s). In some embodiments, the term “protease” as used herein refer to any one or more protease(s) expressed by a species of Bacteroides, such as one or more of Bacteroides vulgatus, Bacteroides dorei, Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, any other Bacteroides species. In one embodiment, the term “protease” as used herein refer to any one or more protease(s) expressed by Bacteroides vulgatus and/or Bacteroides dorei. In some embodiments, the protease is a host protease (i.e., a protease expressed by a subject but not the bacteria in the subject), such as trypsin.

As used herein, a target of a protease refer to a protein, a peptide, a fragment of each thereof, or a molecule comprising such a protein, peptide or fragment, which can be broken-down by the protease into smaller fragments and/or single amino acids. Such target can be identified and quantified by a method as disclosed herein, such as a metabolome method as disclosed in the Experimental Example No. 2, thus resulting in a number of various targets and abundance of one or more or all of targets, respectively. In some embodiments, a target can be a peptide or a fragment thereof, such as a dipeptide.

The term “protein”, “peptide” and “polypeptide” are used interchangeably and in their broadest sense to refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide must contain at least two amino acids and no limitation is placed on the maximum number of amino acids which can comprise a protein's or peptide's sequence. As used herein the term “amino acid” refers to either natural and/or unnatural or synthetic amino acids, including glycine and both the D and L optical isomers, amino acid analogs and peptidomimetics. In some embodiments, the peptide comprises or consists essentially of, or yet further consists of a dipeptide. In some embodiments, the peptide comprises or consists essentially of, or yet further consists of an oligopeptide.

As used herein, the term “dipeptide” refers to an organic compound derived from two amino acids, such as a peptide comprising or consisting essentially of or yet further consisting of two amino acid residues. Non-limiting examples include Aspartame (N-L-a-aspartyl-L-phenylalanine 1-methyl ester), Carnosine (beta-alanyl-L-histidine), Anserine (beta-alanyl-N-methyl histidine), Acetylcarnosine, L-Tryptophan, Phe-Ile, Phe-Pro, Tyr-Pro, Glu-Phe, Ala-Gln and/or Gly-Tyr, Val-Tyr, Homoanserine (N-(4-aminobutyryl)-L-histidine), Kyotorphin (L-tyrosyl-L-arginine), Balenine [ja] (or ophidine) (beta-alanyl-N tau-methyl histidine), Glorin (N-propionyl-γ-L-glutamyl-L-ornithine-δ-lac ethyl ester), Barettin (cyclo-[(6-bromo-8-en-tryptophan)-arginine]), Pseudoproline, and/or Dialanine. The constituent amino acids can be the same or different. When different, two isomers of the dipeptide are possible, depending on the sequence. In some embodiments, the dipeptide is selected from one or more of: L-Tryptophan, Phe-Ile, Phe-Pro, Tyr-Pro, and/or Glu-Phe.

As used herein, the term “oligopeptide” refers to a peptide comprising or consisting essentially of, or yet further consisting of about two to about fifty (such as about two to about twenty) amino acid residues, such as a dipeptide, a tripeptide, a tetrapeptides and a pentapeptide.

An activity of one or more protease can be measured, for example via applying the protease to its target(s) and measuring abundance changes of the target(s). In some embodiments, breaking down the target(s) using a method as disclosed herein generates a detectable signal or removes a detectable signal. More details can be found in the Experimental Examples as disclosed herein. Such protease activity can be one or more of the following: a serine-type endopeptidase activity, a serine-type peptidase activity, a metalloendopeptidase activity, a metal ion binding activity, a hydrolase activity, a dipeptidyl-peptidase activity, a serine-type aminopeptidase activity, a zinc ion binding activity, an aminopeptidase activity, and/or a metallopeptidase activity.

As used herein, the term abundance of a molecule, such as a peptide, refers to quantity of such molecule. Such quantity can be illustrated in the context of a sample, as a concentration of the molecule in the sample and/or a total number and/or weight of the molecule in the sample. Additionally or alternatively, such quantity can have be normalized, such as by a quantity of another molecule in the same sample or a different sample, or by a physical property of the sample (e.g., weight). In one non-limiting example, the abundance of a target and/or a protease can be the quantity of the target and/or protein in a sample (such as a fecal sample) normalized by a protein in the same or another sample (e.g., a serum sample), such as calprotectin. In another non-limiting example, the abundance of a target and/or a protease can be the quantity of the target and/or protein in a sample normalized by weight of the sample. In yet another non-limiting example, the abundance of a target and/or a protease can be the quantity of the target and/or protein in a sample normalized by another target and/or protein in the same sample, such as a quantity of a Bacteroides vulgatus protease normalized by a quantity of a Bacteroides theta protease or a quantity of a target or protein in a fecal sample normalized by the calprotectin quantity in the same sample.

A protease inhibitor decreases, reduces or inhibits an activity of one or more protease(s). Such inhibitors can be classed based on the proteases, such as a serine protease inhibitor, a cysteine protease inhibitor, a metalloprotease inhibitor, an aspartic protease inhibitor, a threonine protease inhibitor, a trypsin inhibitor, and/or an aspartyl protease inhibitor. Several families of inhibitors have been identified, such as Inhibitor 14, 19, I10, 124, 129, 134, 136, 142, 148, 153, 167, 168, and/or 178. An inhibitor as used herein can refer to any one or more of the class(es) and/or families. Further, an inhibitor can be a naturally occurring protease inhibitor, such as SerpinA1 and SerpinA3, or an artificial one, such as water-solubilized 4(2-Aminoethyl)benzenesulfonyl Fluoride (AEBSF, MP Biomedicals), water-solubilized E-64 (Sigma), DMSO-solubilized GM6001 (EMD Millipore), DMSO-solubilized Pepstatin A (MP Biomedicals), and one or more or all inhibitors in Roche cOmplete EDTA-free protease inhibitor cocktail (Sigma, see, for example, www.sigmaaldrich.com/catalog/product/roche/coedtafro?lang=en&region=US, which is enclosed herein by reference in its entirety). More examples can be found at, e.g., www.drugs.com/drug-class/protease-inhibitors.html, go.drugbank.com/unearth/q?utf8=%E2%9C%93&searcher=drugs&query=Protease+inhibitor, and www.sigmaaldrich.com/life-science/biochemicals/biochemical-products.html?TablePage=15649049.

AEBSF, also referred to as 4-(2-aminoethyl)benzenesulfonyl fluoride hydrochloride is a water-soluble, irreversible serine protease inhibitor with a molecular weight of 239.5 Da. It inhibits proteases like chymotrypsin, kallikrein, plasmin, thrombin, and trypsin. The specificity is similar to the inhibitor PMSF, nevertheless AEBSF is more stable at low pH values. Typical usage is 0.1-1.0 mM.

E-64 is an epoxide which can irreversibly inhibit a wide range of cysteine peptidases, such as papain, cathepsin B, cathepsin L, calpain and staphopain. The low toxic effects of the inhibitor, in addition to its effective mechanism of action, makes E-64 a potential template for drugs to treat diseases where high levels of a cysteine proteases are the primary cause. See its structure below.

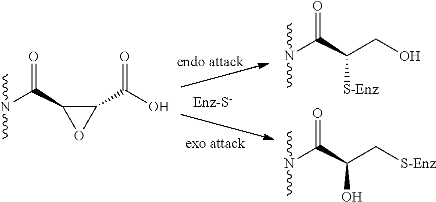

GM6001, also referred to as Ilomastat, is a broad-spectrum matrix metalloproteinase inhibitor, a structure of which is provided below. It is a member of the hydroxamic acid class of reversible metallopeptidase inhibitors. Examples of enzymes that ilomastat inhibit include rabbit MMP9, thermolysin, peptide deformylase, and anthrax lethal factor endopeptidase (LF) produced by the bacterium Bacillus anthracis.

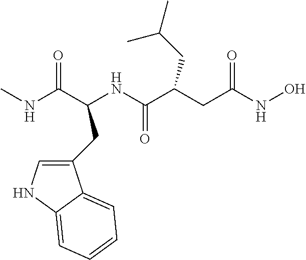 [SMILES: CNC(=O)[C@H](Cc1c[nH]c2ccccc12)NC(=O)[C@@H](CC(=O)NO)CC(C)C]

Pepstatin is a potent inhibitor of aspartyl proteases. It is a hexa-peptide containing the unusual amino acid statine (Sta, (3S,4S)-4-amino-3-hydroxy-6-methylheptanoic acid), having the sequence Isovaleryl-Val-Val-Sta-Ala-Sta (Iva-Val-Val-Sta-Ala-Sta, SEQ ID NO: 3). It was originally isolated from cultures of various species of Actinomyces due to its ability to inhibit pepsin at picomolar concentrations. Pepstatin A is well known to be an inhibitor of aspartic proteinases such as pepsin, cathepsins D and E. Except for its role as a proteinase inhibitor, however, the pharmacological action of pepstatin A upon cells remain unclear. Pepstatin is practically insoluble in water, chloroform, ether, and benzene, however it can be dissolved in methanol, ethanol, and DMSO with acetic acid, to between 1 and 5 mg/ml.

A conventional treatment refers to one or more treatment(s) known and/or used and/or accepted by a health professional. In some embodiments, it can comprise or consist essentially of, or yet further consist of a treatment widely used and accepted by most health professional. Additionally or alternatively, it can comprise or consist essentially of, or yet further consist of an alternative treatment, which are not as widely used. In some embodiments, a conventional treatment for a disease as disclosed herein can comprise or consist essentially of, or yet further consist of a non-specific immunosuppressive (IS) agent, optionally selected from a seroid or a thiopurine. Additionally or alternatively, a conventional treatment for a disease as disclosed herein can comprise or consist essentially of, or yet further consist of treatment with one or more of the following: 5-ASA (also referred to as 5 aminosalicylate therapy, widely used in the management of mild to moderate IBD), an IS (for example, azathioprine, 6-mercaptopurine, methotrexate, cyclosporine A, tacrolimus), a biologic (for example, a TNF antagonist, Vedolizumab (n=3), and/or tofacitinib). See, more details at for example, www.crohnscolitisfoundation.org/sites/default/files/legacy/assets/pdfs/immunomodulators.pdf, which is enclosed herein by reference in its entirety. Additionally or alternatively, a conventional treatment can comprise, or consist essentially of, or yet further consist of one or more of antibiotic (such as one killing a pathogenic bacteria) and/or microbiota not comprising a pathogenic bacteria, such as via a fecal transplantation and/or transplanting a composition comprising the microbiota.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As used herein, the tern alter or any grammatical variation thereof means that a change can be identified. In some embodiments, such change can comprise or consist essentially of, or yet further consist of a change in abundance (increased or decreased). Additionally or alternatively, such change can comprise or consist essentially of, or yet further consist of a change in one or more of the following: location (such as in a cell and/or a tissue and/or an organ and/or a subject), shape, and/or function (such as breaking down a protease target, preventing leakage, and/or sealing paracellular pathway).

Tight junctions, also known as occluding junctions or zonulae occludentes (singular, zonula occludens) are multiprotein junctional complexes whose general function is to prevent leakage of transported solutes and water and seals the paracellular pathway. Tight junctions can also serve as leaky pathways by forming selective channels for small cations, anions, or water. Tight junctions are present mostly in vertebrates (with the exception of Tunicates). The corresponding junctions that occur in invertebrates are septate junctions. As used herein, a tight junction protein refers to a protein, a peptide, and/or a fragment thereof forming a tight junction. Non-limiting examples of tight junction proteins include occludin, zonula occludens-1 (ZO-1), claudin, and Junction Adhesion Molecules (JAM). Occludin is an enzyme (EC 1.6) that oxidizes NADH. It was first identified in epithelial cells as a 65 kDa integral plasma-membrane protein localized at the tight junctions, and together with Claudins, and zonula occludens-1 (ZO-1), has been considered a staple of tight junctions. Claudins were discovered after occludin and are a family of 24 different mammalian proteins. They have a molecular weight of ˜20 kDa. They have a structure similar to that of occludin in that they have four transmembrane domains and similar loop structure. They are understood to be the backbone of tight junctions and play a significant role in the tight junction's ability to seal the paracellular space. Different claudins are found in different locations throughout the human body. Junction Adhesion Molecules (JAM) are part of the immunoglobulin superfamily. They have a molecular weight of ˜40 kDa. Their structure differs from that of the other integral membrane proteins in that they only have one transmembrane protein instead of four. It helps to regulate the paracellular pathway function of tight junctions and is also involved in helping to maintain cell polarity.

The gastrointestinal wall of the gastrointestinal tract is made up of four layers of specialized tissue. From the inner cavity of the gut (the lumen) outwards, these are: Mucosa, Submucosa, Muscular layer, and Serosa or adventitia. The mucosa is the innermost layer of the gastrointestinal tract. It surrounds the lumen of the tract, and comes into direct contact with digested food (chyme). The mucosa itself is made up of three layers: the epithelium, where most digestive, absorptive and secretory processes occur; the lamina propria, a layer of connective tissue, and the muscular is mucosae, a thin layer of smooth muscle. The epithelial cell layer is selectively permeable to bacterial metabolites and digested nutrients allowing regulated transport of soluble molecules through the paracellular space between the epithelial cells. Such selectively permeable ability is referred to herein as permeability. Paracellular transport is controlled by intercellular tight junction (TJ) structures. Disruption of the intestinal TJ barrier, followed by permeation of lumenal noxious molecules, induces a perturbation of the mucosal immune system and inflammation, and can act as a trigger for the development of intestinal and systemic diseases. Further, the shape of epithelial cell, for example, in a planar epithelial monolayer can be used as an indicator of one or both of: intactness of the epithelium and/or its permeability. In some embodiment, a higher circularity of a cell (i.e., a rounder cell) indicates a better intactness of the epithelium and/or a lower permeability of the epithelial cell layer.

As used herein, the term “sample” and “biological sample” are used interchangeably, referring to sample material isolated from or derived from a subject. Biological samples can include tissues, cells, protein or membrane extracts of cells, and biological fluids (e.g., ascites fluid or cerebrospinal fluid (CSF)) isolated from the subject, e.g., the GI of the subject, as well as tissues, cells and fluids present within a subject. Biological samples can include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the rectum, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, tears and stool. In some embodiments, the sample is a serum sample. In some embodiments, the sample is a fecal sample. In some embodiments, the sample is a mucosal and/or GI biopsy, optionally from the colon. Additional examples of samples are provided in the Experimental Examples, infra. In some embodiments, the sample is collected from a subject. In further embodiments, such collected sample can be further processed to generate a sample for use. Non-limiting examples of such processing include one or more of: purification, isolation, concentration and/or removal of certain components.

A “composition” is intended to mean a combination of active agent, such as a protease inhibitor as disclosed herein, and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. For examples of carriers, stabilizers and adjuvants, see Martin (1975) Remington's Pharm. Sci., 15th Ed. (Mack Publ. Co., Easton).

“Pharmaceutically acceptable carriers” refers to any diluents, excipients or carriers that can be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like and consistent with conventional pharmaceutical practices.

A “biocompatible scaffold” refers to a scaffold or matrix. In other embodiments, a biocompatible scaffold is a precursor to an implantable device which has the ability to perform its intended function, with the desired degree of incorporation in the host, without eliciting an undesirable local or systemic effects in the host. Biocompatible scaffolds are described in U.S. Pat. Nos. 6,638,369 and 8,815,276.

“Administration” intends the delivery of a substance to a subject such as an animal or human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration is local or systemical, including oral administration, enteral administration, rectal administration, urogenital (such as vaginal) administration, nasal administration (inhalation), injection (such as intravenous or intramuscular), topical application, by suppository, as a spray (aerosol administration), dry application, or as a solute (for admixing with an aqueous environment).

The terms or “acceptable,” “effective,” or “sufficient” when used to describe the selection of any components, ranges, dose forms, etc. disclosed herein intend that said component, range, dose form, etc. is suitable for the disclosed purpose.

The term “effective amount” refers to a quantity sufficient to achieve a beneficial or desired result or effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of a therapeutic composition, in some embodiments the effective amount is the amount sufficient to result in a protective response against a pathogen or alternatively to support a healthy state of being. In some embodiments, the amount is sufficient to accomplish one or more of 1) clear pathogen; 2) restore healthy microbiota; 3) modulate the immune system; 4) maintain metabolism and metabolic pathways; and 5) reduce toxic compounds in the environment (toxic compounds in water, soil, air, and compounds such as heavy metals (e.g., chromium, arsenic, mercury, radioactive actinides, uranium, plutonium, thorium, polycyclic aromatic hydrocarbons (PAH), petroleum hydrocarbon, crude oil, refined oil, herbicide contamination or pesticide contamination).

In some embodiments, the amount is sufficient to accomplish one or more of a) reduce level (such as expression level) of a protease peptides expressed by a pathogenic bacteria; b) reduce an activity of a protease expressed by a pathogenic bacteria; c) reduce a level (such as expression level, abundance, total amount, and/or total numbers of those having sequences different from each other) of a peptide as disclosed herein; d) correcting expression (such as expression level and/or expression pattern) of a tight junction protein; e) increase epithelial cell circularity; f) decrease permeability of an epithelial cell layer; g) reduce an inflammation (such as a chronic and/or recurrent inflammation; and/or h) treating or preventing a disease as disclosed herein, such as those resistant to a conventional treatment.

In the case of an in vitro or ex vivo applications, in some embodiments the effective amount will depend on the size and nature of the application in question. It will also depend on the nature and sensitivity of the in vitro target and the methods in use. The skilled artisan will be able to determine the effective amount based on these and other considerations. Suitable in vivo dosages and effective amount can be determined using techniques that convert in vitro or dosages in a suitable animal model to use in an animal. The effective amount can comprise one or more administrations of a composition depending on the embodiment.

The agents and compositions can be used in the manufacture of medicaments and for the treatment of humans and other animals by administration in accordance with conventional procedures, such as an active ingredient in pharmaceutical compositions.

An agent or composition of the present disclosure can be administered for therapy by any suitable route of administration. It will also be appreciated that the preferred route will vary with the condition and age of the recipient and the disease being treated.

Exosomes are membrane-bound extracellular vesicles (EVs) that are produced in the endosomal compartment of most eukaryotic cells. In multicellular organisms, exosomes and other EVs were discovered in biological fluids including blood, urine, cerebrospinal fluid, and stool⁸⁵. Since the size of exosomes is limited by that of the parent multivesicular body (MVB), exosomes are generally thought to be smaller than most other EVs, from about 30 to 150 nanometres (nm) in diameter: around the same size as many lipoproteins but much smaller than cells.

### MODES FOR CARRYING OUT THE DISCLOSURE

Ulcerative colitis (UC) has a significant global burden¹, and is characterized by an aberrant immune response directed towards the gut microbiota². Current treatment options exclusively target host inflammatory pathways and are often ineffective in managing disease³. Genomic technologies have identified associations between microbial dysbiosis, or temporal shifts in composition, and UC severity^{2,4,5}. Associations exist between the microbiome and Ulcerative Colitis^{81,82}. Targeting the microbiome through fecal material transplant has been demonstrated to be effective in inducing clinical and endoscopic remission. These strategies all approach the broad targeting of the microbial community. Broadly targeting the microbial community is then associated with risk (obesity, diabetes, infections). It is also demonstrated that specific bacteria and metabolites drive efficacy of fecal transplant⁸³. While recent efforts extended profiling of microbiota in UC beyond genomics⁶, it remains poorly understood if these shifts are causal or associative in nature, and which mechanisms govern pathogenic roles of the microbiome in UC. Metaproteomics is a developing mass spectrometry (MS) method for the comprehensive analysis of the proteins expressed by a community of organisms⁷. Applicant has identified specific bacterial sources of persistent disease activity in ulcerative colitis. Applicant's integration of a contemporary metaproteomics platform with other technologies allowed for a more in-depth understanding of host-microbiome interactions governing UC severity and the identification of novel microbial therapeutic targets⁸⁻¹¹.

In line with this studies, Applicant collected and analyzed six fecal or serum based—omic datasets from 40 UC patients displaying a wide range of clinical, endoscopic, and histologic disease activity. Additionally, meta-omics were collected on a second cohort of 210 patients including 73 UC, 117 Crohn's disease (31 colonic, 32 Ileal and 34 Ileocolonic), and 20 healthy controls. Several multi-omics analyses were performed, including, for example, 16S gene amplicon sequencing, metagenomics, metabolomics, metaproteomics (such as using serum and/or fecal samples), and metapeptidomics. All meta-omics displayed large-scale shifts related to disease activity, with the metabolome and metaproteome best predicting disease severity. Overproduction of Bacteroides vulgatus peptidases correlates to increased UC disease activity. Over producers show more peptidases than expected from DNA while under producers display less peptidases than expected from DNA. Leveraging multiple data layers, a subset of ˜40% of clinically active UC patients were identified as having unexpected over-abundance of Bacteroides proteases. The supernatant of Bacteroides vulgatus, prominent in the metagenome of the UC patients, contained active serine proteases. Metabolomic support for proteolysis hypothesis. Protease inhibition was shown to improve Bacteroides vulgatus induced reductions in intestinal epithelial permeability in vitro, and prevent histological colitis in Bacteroides vulgatus monocolonized mice. Colonic cell monolayers are disrupted by Bacteroides vulgatus. Protease inhibition prevents Bacteroides vulgatus penetration of colonic cell monolayers. Protease inhibition prevents B. vulgatus driven colitis in mice. Furthermore, transplantation of fecal material from UC patients into germ-free mice resulted in colitis, and oral administration of protease inhibitors attenuated disease severity. As further detailed in the Experimental Examples, Applicant showed that Bacteroides proteases are a source of persistent mucosal inflammation in Ulcerative Colitis. Blocking these proteases prevented and attenuated: Bacteroides induced disruption of epithelial barrier function in Caco-2 cells; Bacteroides induced microscopic colitis in monocolonization of gnotobiotic mice; Fecal transplant induced macroscopic colitis in gnotobiotic mice. Excessive protease production by Bacteroides is seen in a sub-set of patients with persistent disease activity despite conventional medical therapy (40-45% of population). Thus, targeting Bacteroides proteases can represent a novel personalized therapeutic target for Ulcerative Colitis patients not responding to conventional medical therapy or a primary therapy in preventing the development of colitis for at-risk individuals.

In sum, Applicant's findings indicate that serine-protease inhibition of Bacteroides vulgatus proteases represents a novel therapeutic approach for UC and related disorders and diseases. 3. The most significant changes implemented were the depth of proteomics sequencing and analyses we did compared to those prior to us, and the specific manner in which we used computational tools and analyses to identify this discovery

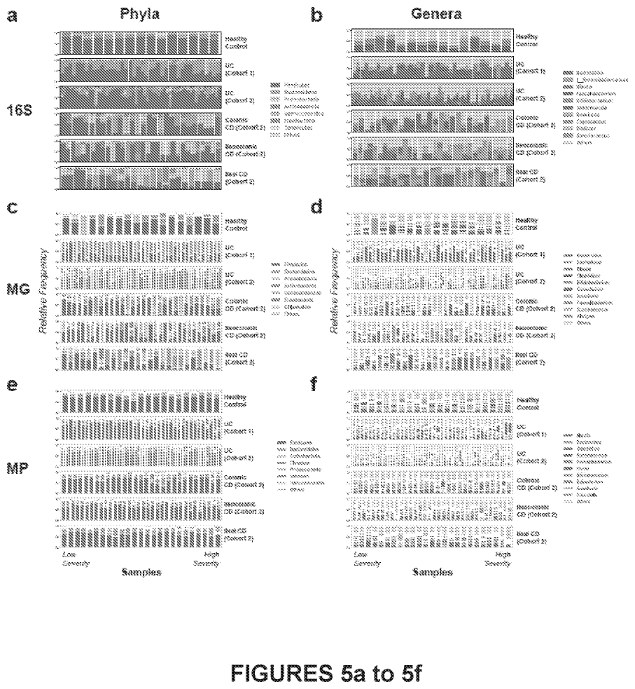

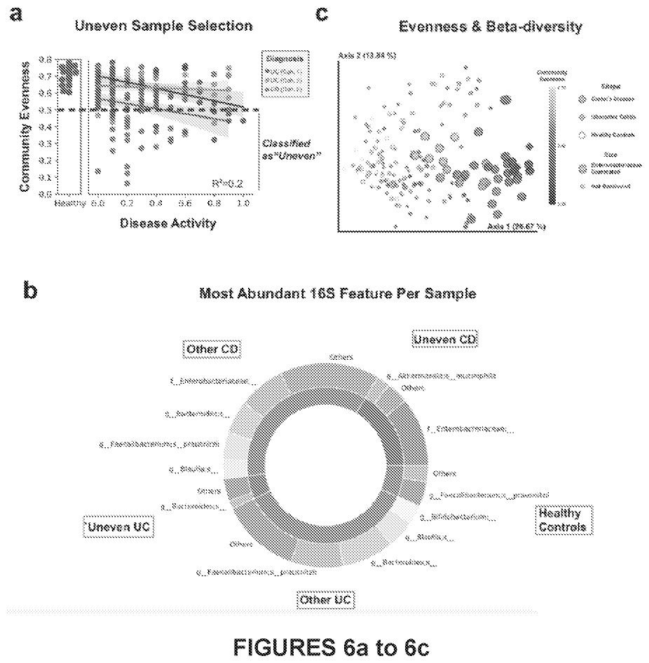

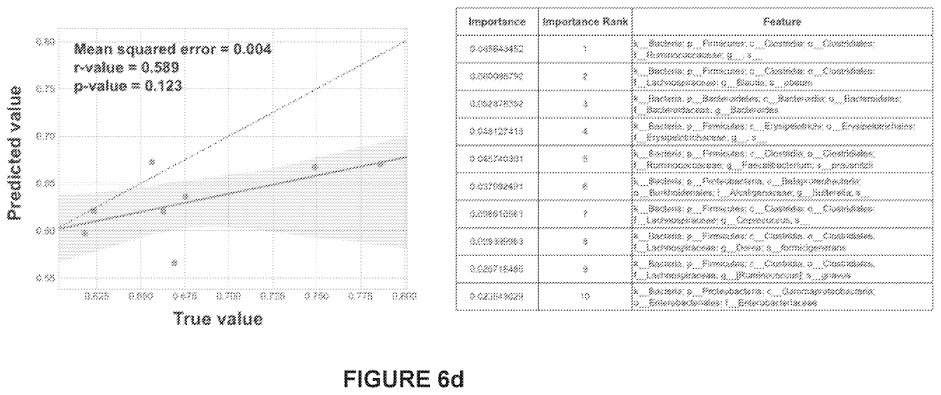

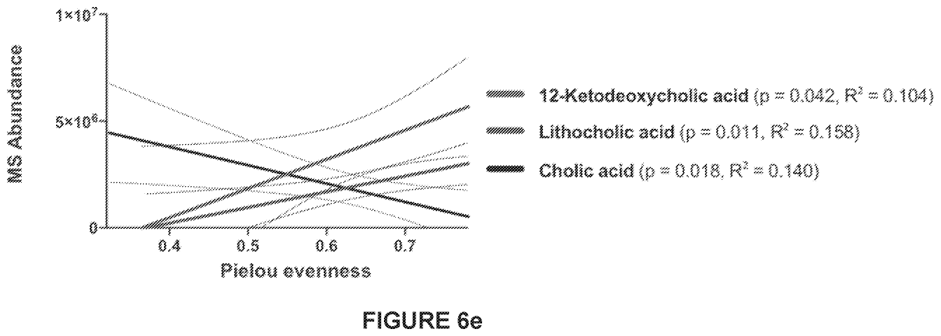

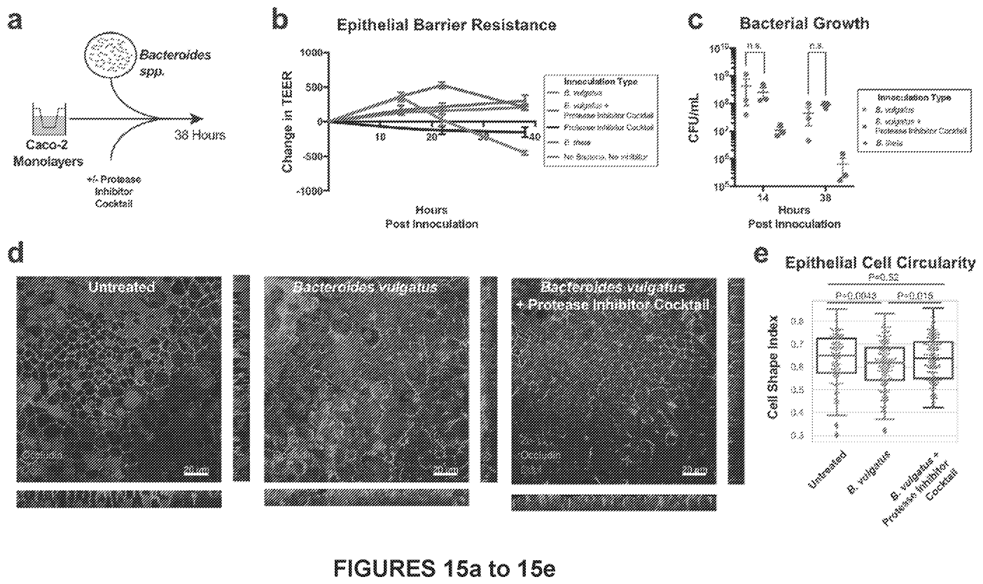

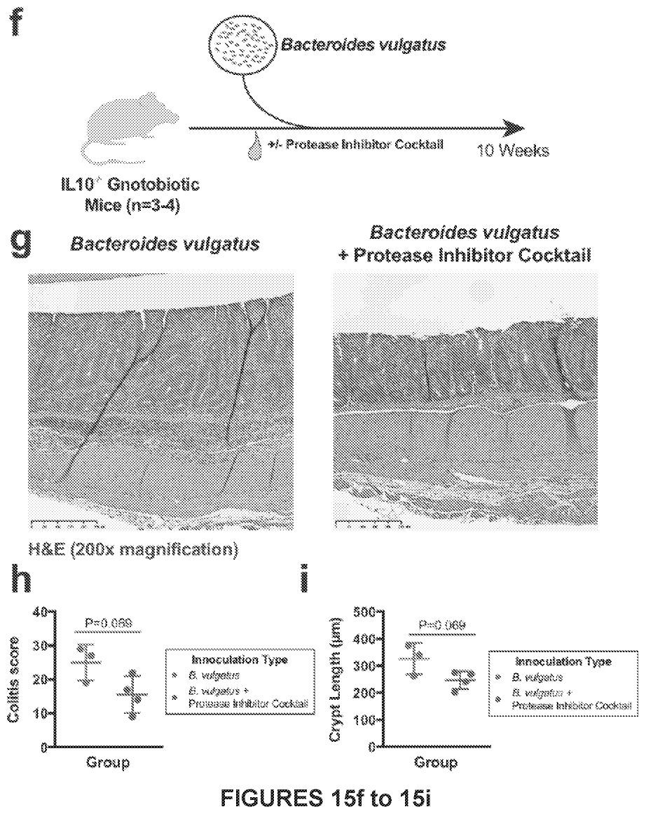

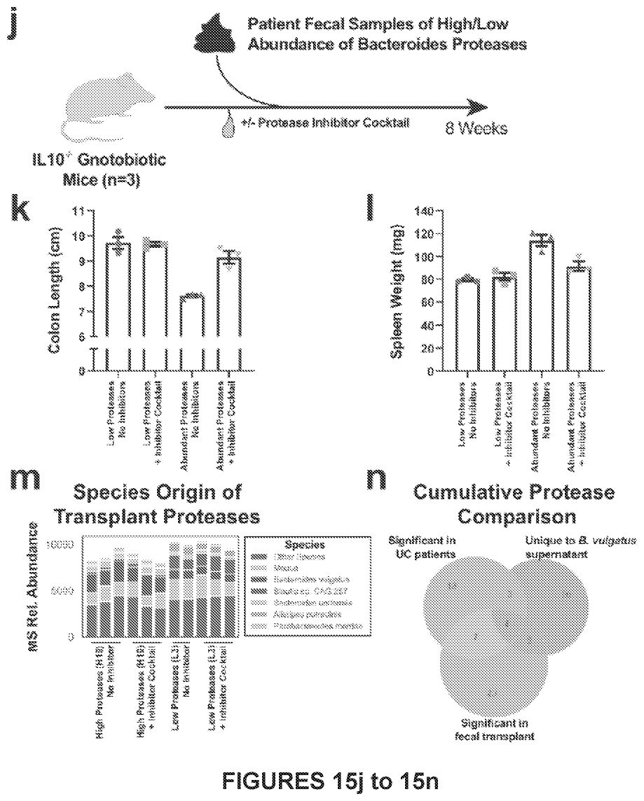

In some embodiments, the proteases are expressed by one or more of the organisms as identified herein, such as those listed in FIGS. 5a to 5f, 6b, 6d 7a, 7b, 7d, 8a-8e, 9a, 9b, 14a, and 15m and/or Table 3. In some embodiments, the organism is a Bacteroides organism, e.g., one or more of: Bacteroides vulgatus, Bacteroides dorei, Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, or any other Bacteroides species. In some embodiments, the organism is Bacteroides vulgatus and Bacteroides dorei. In some embodiments, the organism is Bacteroides vulgatus. In some embodiments, the organism is Bacteroides dorei. In some embodiments, the organism does not comprise one or more of: Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, or any other Bacteroides species except Bacteroides vulgatus or Bacteroides dorei. In some embodiments, the organism does not comprise Bacteroides theta. Additionally or alternatively, the organism does not comprise Bacteroides fragilis. In some embodiments, the protease may be expressed by other organisms, such as those not expressed by the pathogenic organism(s) but expressed by a subject who does or does not comprises the pathogenic organism(s), and/or those expressed by an organisms as disclosed in FIG. 15m. In other embodiments, the protease is not either or both of: a protease not expressed by the pathogenic organism(s) but expressed by a subject who does or does not comprises the pathogenic organism(s), and/or those expressed by an organisms as disclosed in FIG. 15m.

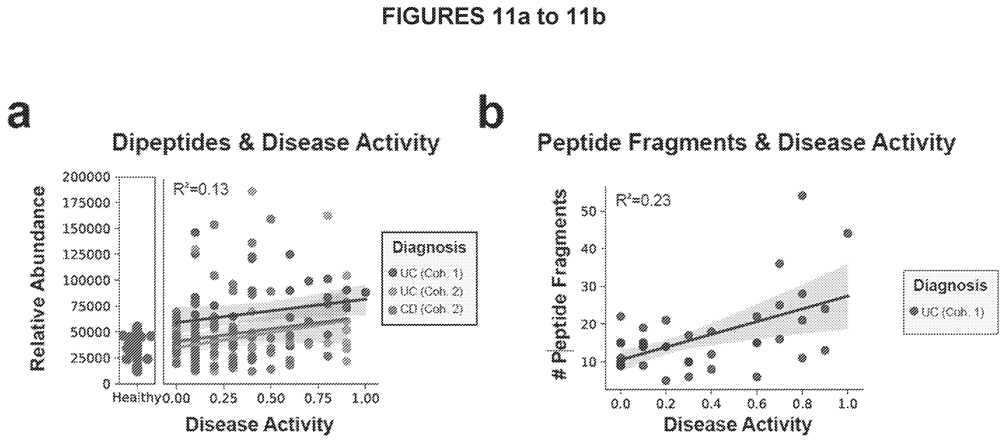

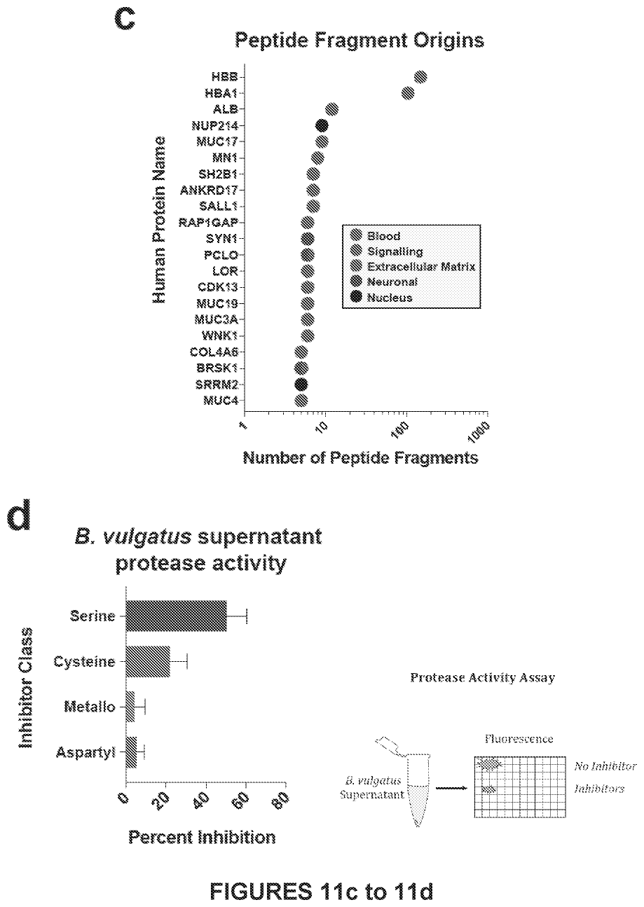

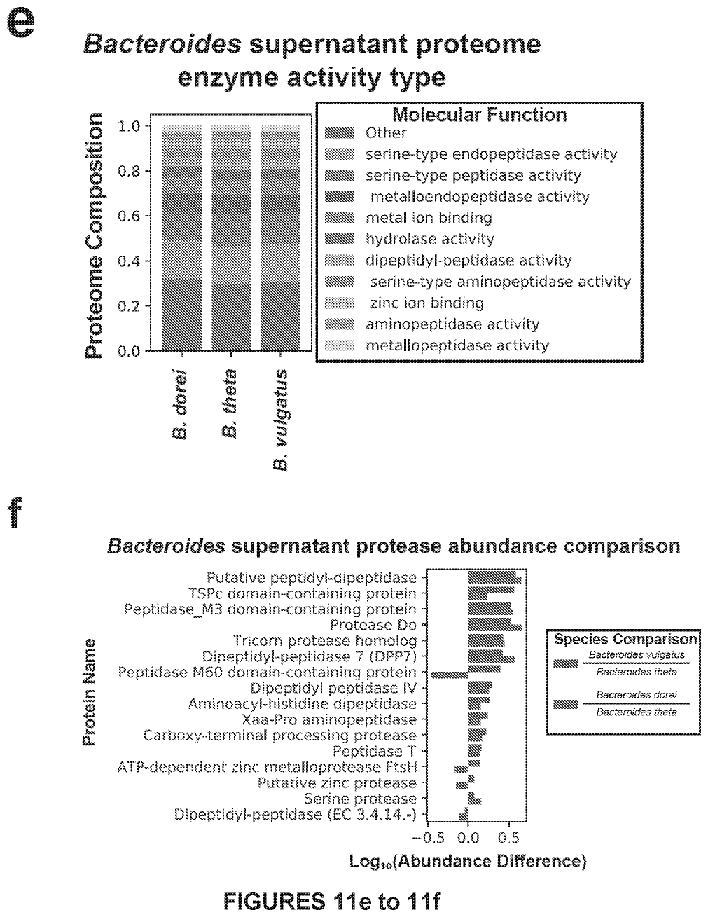

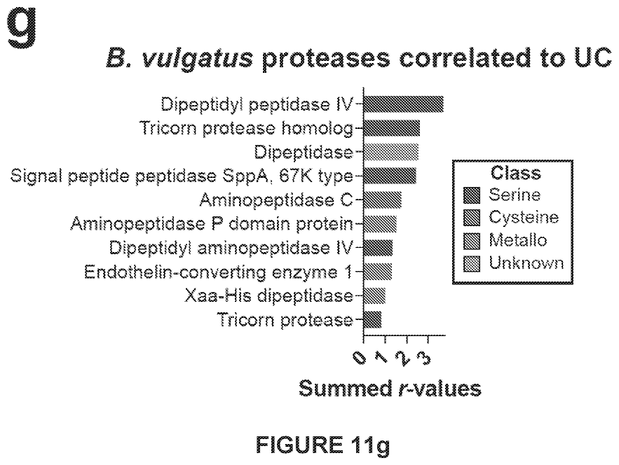

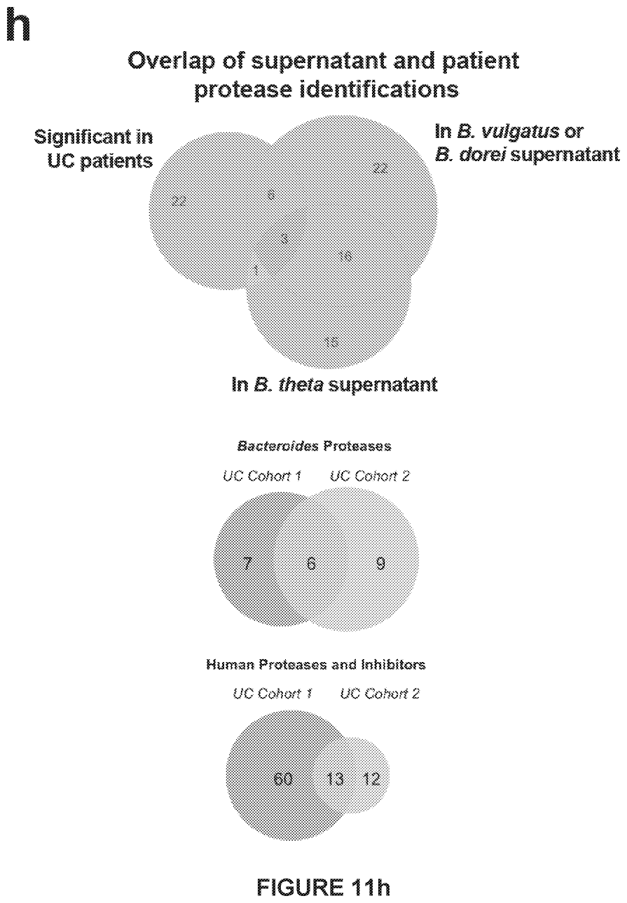

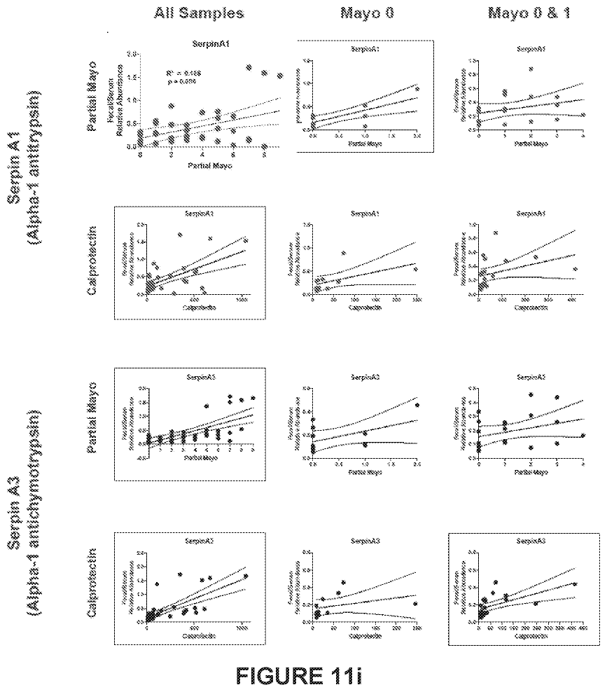

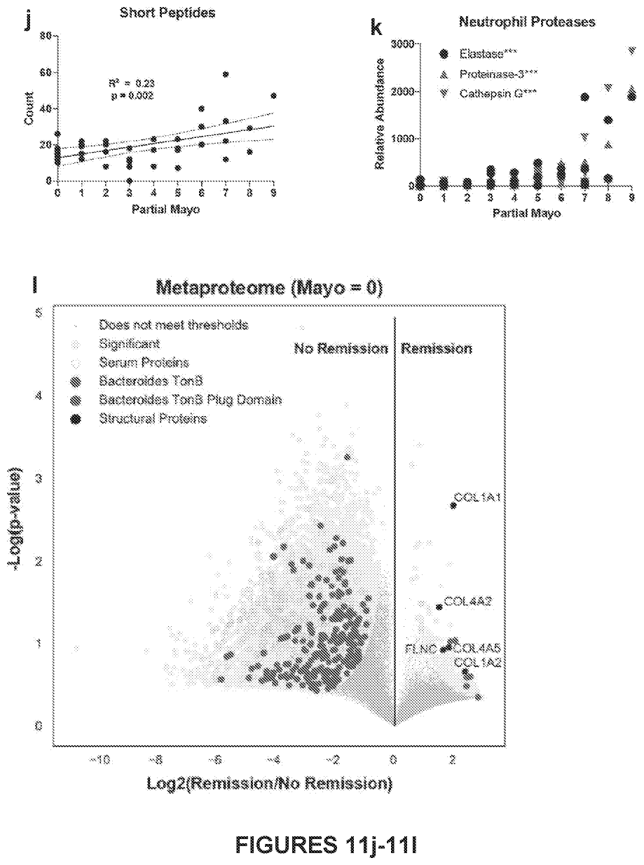

In some embodiments, the protease is selected from one or more of those as disclosed herein, such as one or more of those in FIGS. 7e, 11e, 11f, 11g, and/or 18a-18e and/or those identified in Table 4. In further embodiments, the protease is selected from those disclosed more than once in these Figures and Table of this disclosure. In some embodiments, the protease is a Bacteroides bacterium protease. Additionally or alternatively, the protease is selected from one or more of a serine protease, a metalloproteinase, an aspartyl protease and a cysteine-protease. In some embodiments, the protease is selected from one or both of serine protease and/or cysteine protease. In some embodiments, the protease is a serine protease. In other embodiments, the protease is a cysteine protease. In some embodiment, the protease is selected from one or more of the following: one or more serine protease(s) including but not limited to dipeptidase (EC 3.4.-.-), dipeptidyl aminopeptidase IV, dipeptidyl peptidase IV, dipeptidyl peptidase IV N-terminal domain protein (fragment), dipeptidyl peptidase 7 (DPP7) (EC 3.4.14.-), dipeptidyl peptidase III (EC 3.4.14.4), peptidase S9A/B/C family catalytic domain protein, peptidase S9A/B/C family catalytic domain protein (EC 3.4.-.-), prolyl oligopeptidase family protein, prolyl tripeptidyl peptidase (EC 3.4.14.12), Protease Do, putative tricorn-like protease, serine protease, signal peptidase I (EC 3.4.21.89), Signal peptide peptidase SppA, 67K type (EC 3.4.-.-), tricorn protease, and tricorn protease homolog (EC 3.4.21.-); one or more metalloprotease(s), including but not limited to aminopeptidase P domain protein (EC 3.4.-.-), endothelin-converting enzyme 1 (EC 3.4.24.71), leucine aminopeptidase, peptidase M16 inactive domain protein, peptidase M16 inactive domain protein (EC 3.4.24.-), peptidase M24 family (EC 3.4.-.-), peptidase M28 family (EC 3.4.-.-), putative endothelin-converting enzyme, putative peptidyl-dipeptidase dcp, TldD/PmbA family protein, Xaa-His dipeptidase (EC 3.4.13.3), Xaa-Pro aminopeptidase (EC 3.4.11.9); and/or one or more cysteine protease(s), including but not limited to aminopeptidase C, aminopeptidase C (Bleomycin hydrolase), peptidase C1-like family protein (Fragment), peptidase C1A papain, aminoacyl-histidine dipeptidase (Cytosol non-specific dipeptidase), carboxypeptidase regulatory-like domain protein, carboxypeptidase (TonB-dependent receptor plug domain protein) and cytosol non-specific dipeptidase (EC 3.4.13.18).

In some embodiments, the protease is selected from one or more of the following: one or more or all of putative peptidyl-dipeptidase, TSPc domain-containing protein, peptidase M3 domain-containing protein, protease Do, tricorn protease homolog, dipeptidyl-peptidase 7 (DPP7), peptidase M60 domain-containing protein, dipeptidyl peptidase IV, aminoacyl-histidine dipeptidase, Xaa-Pro aminopeptidase, Carboxy-terminal processing protease, peptidase T, ATP-dependent zinc metalloprotease FtsH, putative zinc protease, serine protease, dipeptidyl-peptidase (EC 3.4.14.-); and/or one or more or all of dipeptidyl peptidase IV, tricorn protease homolog, dipeptidase, signal peptide peptidase SppA 67K type, aminopeptidase C, aminopeptidase P domain protein, Dipeptidyl aminopeptidase IV, endothelin-converting enzyme 1, Xaa-His dipeptidase, tricorn protease. In some embodiments, the protease is selected from one or more of dipeptidyl peptidases (e.g. DPPIV, and/or DPPVII).

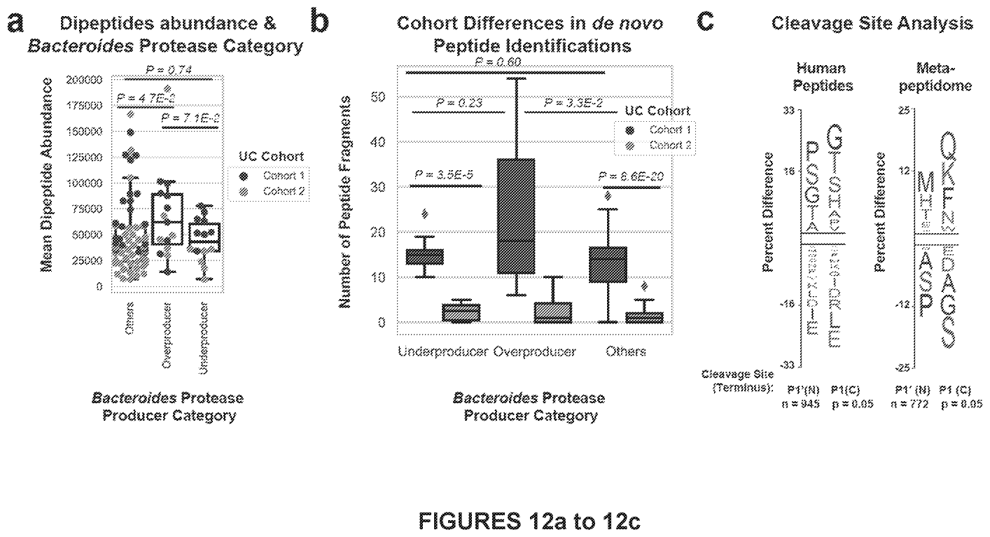

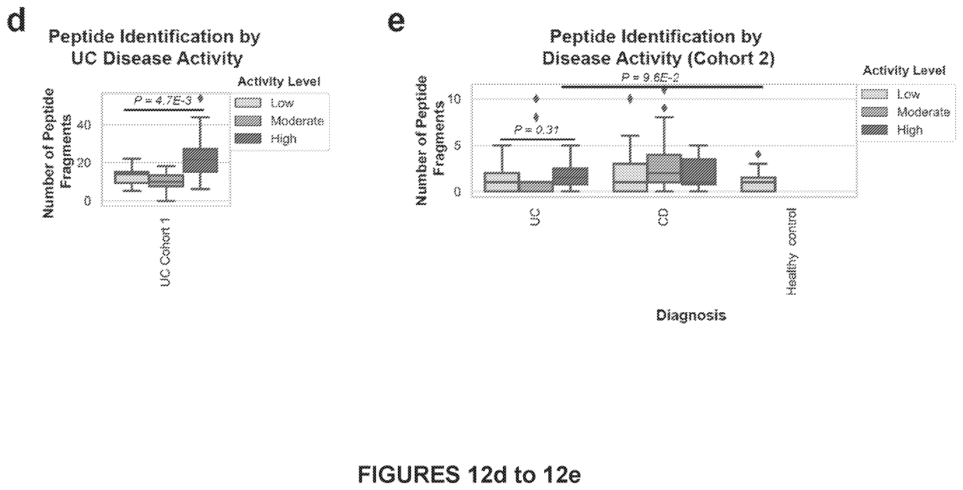

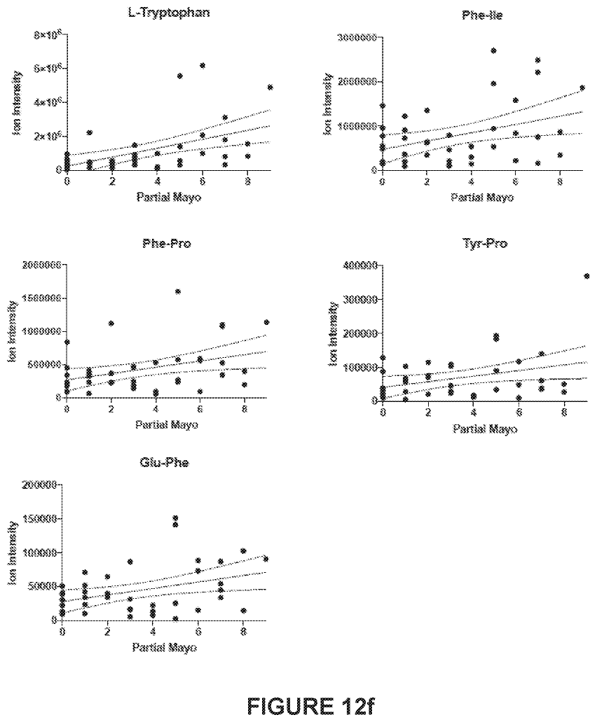

In some embodiments, the protease of the disclosure targets a peptide target and generates fragment(s) of the target after the proteolysis, for example, dipeptide or an oligopeptide. In some embodiments, the target is selected from one or more of a collagen, a mucin, or a peptide identified in FIGS. 11c and 12f, such as HBB (Hemoglobin subunit beta, see, e.g., www.uniprot.org/uniprot/P68871), HBA1 (Hemoglobin subunit alpha 1, see, e.g., www.uniprot.org/uniprot/P69905), ALB (Albumin, see, e.g., www.uniprot.org/uniprot/P02768), NUP214 (Nuclear pore complex protein Nup214, see, e.g., www.uniprot.org/uniprot/P35658), MUC17 (Mucin-17, see, e.g., www.uniprot.org/uniprot/Q685J3), MN1 (Transcriptional activator MN1, see, e.g., www.uniprot.org/uniprot/Q10571), SH2B1 (SH2B adapter protein 1, see, e.g., www.uniprot.org/uniprot/Q9NRF2), ANKRD17 (Ankyrin repeat domain-containing protein 17, see, e.g., www.uniprot.org/uniprot/075179), SALL1 (Sal-like protein 1, see, e.g., www.uniprot.org/uniprot/Q9NSC2), RAP1GAP (Rap1 GTPase-activating protein 1, see, e.g., www.uniprot.org/uniprot/P47736), SYN1 (Synapsin-1, see, e.g., www.uniprot.org/uniprot/P17600), PCLO (Protein piccolo, see, e.g., www.uniprotorg/uniprot/Q9Y6V0), LOR (Loricrin, see, e.g., www.uniprot.org/uniprot/P23490), CDK13 (Cyclin-dependent kinase 13, see, e.g., www.uniprot.org/uniprot/Q14004), MUC19 (Mucin-19, see, e.g., www.uniprot.org/uniprot/Q7Z5P9), MUC3A (Mucin-3A, see, e.g., www.uniprot.org/uniprot/Q02505), WNK1 (Serine/threonine-protein kinase WNK1, see, e.g., www.uniprot.org/uniprot/Q9H4A3), COL4A6 (Collagen alpha-6(IV) chain, see, e.g., www.uniprot.org/uniprot/Q14031), BRSK1 (Serine/threonine-protein kinase BRSK1, see, e.g., www.uniprot.org/uniprot/Q8TDC3), SRRM2 (Serine/arginine repetitive matrix protein 2, see, e.g., www.uniprotorg/uniprot/Q9UQ35), MUC4 (Mucin-4, see, e.g., www.uniprot.org/uniprot/Q99102), L-Tryptopha, Phe-Ile, Phe-Pro, Tyr-Pro, and Glu-Phe. In some embodiments, a target or fragment thereof is selected from one or more of the protein and/or peptide identified in a sample using a method of Metabolomics as disclosed herein, or a fragment thereof. In some embodiments, the target, fragment thereof and/or peptide can be quantified using metapeptidomics.

In some embodiments, protease inhibitor(s) is selected from one or more of those as disclosed herein. In some embodiments, the inhibitor comprises or consists essentially of, or yet further consists of one or more of a serine protease inhibitor, a cysteine protease inhibitor, a metallo protease inhibitor, and/or an aspartyl protease inhibitor. In some embodiments, the inhibitor is selected from the group of: Roche complete EDTA-free protease inhibitor cocktail (Sigma), 4(2-Aminoethyl)benzenesulfonyl Fluoride (MP Biomedical s), Pepstatin A (MP Biomedicals), GM6001 (EMD Millipore) and E-64 (Sigma). Additionally or alternatively, the inhibitor is water-solubilized and/or DMSO-solubilized, such as water-solubilized 4(2-Aminoethyl)benzenesulfonyl Fluoride (AEBSF, MP Biomedicals), water-solubilized E-64 (Sigma), DMSO-solubilized GM6001 (EMD Millipore), and DMSO-solubilized Pepstatin A (MP Biomedicals). In some embodiments, the inhibitor is one or more or all of those in Roche cOmplete EDTA-free protease inhibitor cocktail (Sigma). Additionally or alternatively, the inhibitor may comprise or consist essentially of, or yet further consist of one or more of a naturally occurring inhibitor, such as SerpinA1 and SerpinA3. Additionally or alternatively, the inhibitor reduces and/or inhibits one or more of the following protease activities: a serine-type endopeptidase activity, a serine-type peptidase activity, a metalloendopeptidase activity, a metal ion binding activity, a hydrolase activity, a dipeptidyl-peptidase activity, a serine-type aminopeptidase activity, a zinc ion binding activity, an aminopeptidase activity, and/or a metallopeptidase activity. More examples of inhibitor can be found at, e.g., www.drugs.com/drug-class/protease-inhibitors.html, go.drugbank.com/unearth/q?utf8=%E2%9C%93&searcher=drugs&query=Protease+inhibitor, and www.sigmaaldrich.com/life-science/biochemicals/biochemical-products.html?TablePage=15649049.

In some embodiments, a conventional treatment is a non-specific immunosuppressive agent. In some embodiments, the conventional treatment is selected from one or more of the following: 5-ASA (also referred to as 5 aminosalicylate therapy, widely used in the management of mild to moderate IBD), an IS (for example, azathioprine, 6-mercaptopurine, methotrexate, cyclosporine A, tacrolimus), a biologic (for example, a TNF antagonist, Vedolizumab (n=3), and/or tofacitinib), a seroid or a thiopurine. In some embodiments, the non-specific immunosuppressive agent targeting the host is selected from one or more of, e.g., steroids, thiopurines, and/or biologics.

In some embodiments, a subject as disclosed herein is at risk of having a disease as disclosed herein and/or diagnosed with a disease. Additionally or alternatively, the subject comprises one or more of the protease(s) as disclosed herein, the protease target(s) as disclosed herein, or fragment(s) of each thereof in a sample isolated from the subject. In some embodiments, the sample is a fecal sample. In some embodiments, the subject comprises a higher abundance (such as higher in one or more of: absolute total number, relative total number, and/or number of different peptides) of one or more of the protease(s) as disclosed herein, the protease target(s) as disclosed herein, or fragment(s) of each thereof. In some embodiments, the relative number is calculated as abundance of the protease(s) as disclosed herein, the protease target(s) as disclosed herein, or fragment(s) of each thereof of Bacteroides vulgatus over abundance of the protease(s) as disclosed herein, the protease target(s) as disclosed herein, or fragment(s) of each thereof of Bacteroides theta. In some embodiments, the subject comprises (such as has and/or expresses and/or is detected with) one or more of protein(s) as identified in Table 7, optionally in a sample and further optionally in exosomes of a sample, such as a fecal sample and/or a fecal exosome sample. In some embodiments, the subject has (such as comprises, displays, shows, and/or expresses) one or more of, optionally in a sample as disclosed herein: a high level of a protease as disclosed herein, such as one expressed by the pathogenic bacteria; a high activity of a protease as disclosed herein, such as one expressed by the pathogenic bacteria; a high level of a target or a fragment thereof and/or a peptide; an altered expression of a tight junction protein, such as ZO-1 and/or occludin; a decrease in epithelial cell circularity; an increased permeability of an epithelial cell layer; or resistance to a conventional treatment. In some embodiments, the target or a fragment thereof is a peptide, optionally selected from a dipeptide or an oligopeptide. In some embodiments, the peptide is selected from a target of the protease or a fragment thereof. Additionally or alternatively, the target is selected from one or more of a collagen, a mucin or a peptide as disclosed herein. In some embodiments, the reference relating to the relative terms here is the corresponding level, activity, expression, circularly, permeability and/or resistance of a healthy subject or a subject free of the disease. In some embodiments, the subject has been treated with a conventional treatment. In further embodiments, no disease remission and/or recovery was observed in the subject treated with a conventional treatment (i.e., the subject is resistant to a conventional treatment).

In some embodiments, the sample as used herein is taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the rectum, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, tears and stool. In some embodiments, the sample is a serum sample. In some embodiments, the sample is a fecal sample. In some embodiments, the sample is a mucosal and/or GI biopsy, optionally from the colon, small intestine, rectum or other parts of GI. Additional examples of samples are provided in the Experimental Examples, infra. In some embodiments, the sample is collected from a subject. In further embodiments, such collected sample can be further processed to generate a sample for use. Non-limiting examples of such processing include one or more of: purification, isolation, concentration and/or removal of certain components.

### Compositions

This disclosure provides a composition comprising, or consisting essentially of, or consisting of one or more protease inhibitors, selected to target a protease expressed by an organism (e.g., a pathogenic organism) related to IBD, IFB, UC, CD or a related disease or disorder, alone or in combination with a carrier, e.g., a pharmaceutically acceptable carrier or a biocompatible scaffold. In one aspect, the composition further comprises, or consists essentially thereof, or consists of, a stabilizer or preservative for ease of storage (e.g., freeze and thaw) or administration. In one aspect, the protease inhibitors are optionally combined with one or more of other embodiment(s) and/or aspect(s) as disclosed herein.

The proteases of the compositions are expressed by one or more of the organisms as identified herein, such as those in FIGS. 5a to 5f, 6b, 6d 7a, 7b, 7d, 7g, 7k, 8a-8e, 9a, 9b, 14a, and 15m and/or Table 3. In some embodiments, the organism is a Bacteroides organism, e.g., one or more of: Bacteroides vulgatus, Bacteroides dorei, Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, or any other Bacteroides species. In some embodiments, the organism is Bacteroides vulgatus and Bacteroides dorei. In some embodiments, the organism is Bacteroides vulgatus. In some embodiments, the organism is Bacteroides dorei. In some embodiments, the organism does not comprise one or more of: Bacteroides uniformis, Bacteroides ovatus, Bacteroides fragilis, Bacteroides theta, Bacteroides stercoris, Bacteroides cellulosilyticus, Bacteroides xylanisolvens, Bacteroides caccae, or any other Bacteroides species except Bacteroides vulgatus or Bacteroides dorei. In some embodiments, the organism does not comprise Bacteroides theta. Additionally or alternatively, the organism does not comprise Bacteroides fragilis. In some embodiments, the composition further comprises a protease expressed by another organisms, such as those not expressed by the pathogenic organism(s) but expressed by a subject who does or does not comprises the pathogenic organism(s), and/or those expressed by an organisms as disclosed in FIG. 15m. In other embodiments, the composition does not comprise either or both of: a protease not expressed by the pathogenic organism(s) but expressed by a subject who does or does not comprises the pathogenic organism(s), and/or those expressed by an organisms as disclosed in FIG. 15m.

In another aspect, a protease is disclosed herein, such as one or more of those in FIGS. 7e, 11e, 11f, 11g, and/or 18a-18e and/or those identified in Table 4. In further embodiments, the protease is selected from those disclosed more than once in these Figures and Table of this disclosure. In some embodiments, the protease is a Bacteroides bacterium protease. Additionally or alternatively, the protease is selected from one or more of a serine protease, a metalloproteinase, an aspartyl protease and a cysteine-protease. In some embodiments, the protease is selected from one or both of serine protease and/or cysteine protease. In some embodiments, the protease is a serine protease. In other embodiments, the protease is a cysteine protease. In some embodiment, the protease is selected from one or more of the following: one or more serine protease(s) including but not limited to dipeptidase (EC 3.4.-.-), dipeptidyl aminopeptidase IV, dipeptidyl peptidase IV, dipeptidyl peptidase IV N-terminal domain protein (fragment), dipeptidyl peptidase 7 (DPP7) (EC 3.4.14.-), dipeptidyl peptidase III (EC 3.4.14.4), peptidase S9A/B/C family catalytic domain protein, peptidase S9A/B/C family catalytic domain protein (EC 3.4.-.-), prolyl oligopeptidase family protein, prolyl tripeptidyl peptidase (EC 3.4.14.12), Protease Do, putative tricorn-like protease, serine protease, signal peptidase I (EC 3.4.21.89), Signal peptide peptidase SppA, 67K type (EC 3.4.-.-), tricorn protease, and tricorn protease homolog (EC 3.4.21.-); one or more metalloprotease(s), including but not limited to aminopeptidase P domain protein (EC 3.4.-.-), endothelin-converting enzyme 1 (EC 3.4.24.71), leucine aminopeptidase, peptidase M16 inactive domain protein, peptidase M16 inactive domain protein (EC 3.4.24.-), peptidase M24 family (EC 3.4.-.-), peptidase M28 family (EC 3.4.-.-), putative endothelin-converting enzyme, putative peptidyl-dipeptidase dcp, TldD/PmbA family protein, Xaa-His dipeptidase (EC 3.4.13.3), Xaa-Pro aminopeptidase (EC 3.4.11.9); and/or one or more cysteine protease(s), including but not limited to aminopeptidase C, aminopeptidase C (Bleomycin hydrolase), peptidase C1-like family protein (Fragment), peptidase C1A papain, aminoacyl-histidine dipeptidase (Cytosol non-specific dipeptidase), carboxypeptidase regulatory-like domain protein, carboxypeptidase (TonB-dependent receptor plug domain protein) and cytosol non-specific dipeptidase (EC 3.4.13.18).

Additionally or alternatively, the composition comprises, or consists essentially of, or yet further consists of a protease selected from one or more of the following: one or more or all of putative peptidyl-dipeptidase, TSPc domain-containing protein, peptidase M3 domain-containing protein, protease Do, tricorn protease homolog, dipeptidyl-peptidase 7 (DPP7), peptidase M60 domain-containing protein, dipeptidyl peptidase IV, aminoacyl-histidine dipeptidase, Xaa-Pro aminopeptidase, Carboxy-terminal processing protease, peptidase T, ATP-dependent zinc metalloprotease FtsH, putative zinc protease, serine protease, dipeptidyl-peptidase (EC 3.4.14.-); and/or one or more or all of dipeptidyl peptidase IV, tricorn protease homolog, dipeptidase, signal peptide peptidase SppA 67K type, aminopeptidase C, aminopeptidase P domain protein, Dipeptidyl aminopeptidase IV, endothelin-converting enzyme 1, Xaa-His dipeptidase, tricorn protease. In some embodiments, the protease in the composition comprises or consists essentially of, or yet further consists of one or more of dipeptidyl peptidases (e.g. DPPIV, and/or DPPVII).

In yet another aspect, the protease of the disclosure targets a peptide and generates fragment(s) of the target after the proteolysis, for example, dipeptide or an oligopeptide. In some embodiments, the target is selected from one or more of a collagen, a mucin, or a peptide identified in FIGS. 11c and 12f, such as HBB (Hemoglobin subunit beta, see, e.g., www.uniprot.org/uniprot/P68871), HBA1 (Hemoglobin subunit alpha 1, see, e.g., www.uniprot.org/uniprot/P69905), ALB (Albumin, see, e.g., www.uniprot.org/uniprot/P02768), NUP214 (Nuclear pore complex protein Nup214, see, e.g., www.uniprot.org/uniprot/P35658), MUC17 (Mucin-17, see, e.g., www.uniprot.org/uniprot/Q685J3), MN1 (Transcriptional activator MN1, see, e.g., www.uniprot.org/uniprot/Q10571), SH2B1 (SH2B adapter protein 1, see, e.g., www.uniprot.org/uniprot/Q9NRF2), ANKRD17 (Ankyrin repeat domain-containing protein 17, see, e.g., www.uniprot.org/uniprot/075179), SALL1 (Sal-like protein 1, see, e.g., www.uniprot.org/uniprot/Q9NSC2), RAP1GAP (Rap1 GTPase-activating protein 1, see, e.g., www.uniprot.org/uniprot/P47736), SYN1 (Synapsin-1, see, e.g., www.uniprot.org/uniprot/P17600), PCLO (Protein piccolo, see, e.g., www.uniprot.org/uniprot/Q9Y6V0), LOR (Loricrin, see, e.g., www.uniprot.org/uniprot/P23490), CDK13 (Cyclin-dependent kinase 13, see, e.g., www.uniprot.org/uniprot/Q14004), MUC19 (Mucin-19, see, e.g., www.uniprot.org/uniprot/Q7Z5P9), MUC3A (Mucin-3A, see, e.g., www.uniprot.org/uniprot/Q02505), WNK1 (Serine/threonine-protein kinase WNK1, see, e.g., www.uniprot.org/uniprot/Q9H4A3), COL4A6 (Collagen alpha-6(IV) chain, see, e.g., www.uniprot.org/uniprot/Q14031), BRSK1 (Serine/threonine-protein kinase BRSK1, see, e.g., www.uniprot.org/uniprot/Q8TDC3), SRRM2 (Serine/arginine repetitive matrix protein 2, see, e.g., www.uniprot.org/uniprot/Q9UQ35), MUC4 (Mucin-4, see, e.g., www.uniprot.org/uniprot/Q99102), L-Tryptopha, Phe-Ile, Phe-Pro, Tyr-Pro, and Glu-Phe. In some embodiments, a target or a fragment thereof is selected from one or more of the protein and/or peptide identified in in a sample using a method of Metabolomics as disclosed herein, or a fragment thereof. In some embodiments, the target, fragment thereof and/or peptide can be quantified using metapeptidomics.

In a further aspect, the composition comprises or consists essentially of, or yet further consists of one or more inhibitors as disclosed herein. In some embodiments, the inhibitor comprises or consists essentially of, or yet further consists of one or more of a serine protease inhibitor, a cysteine protease inhibitor, a metallo protease inhibitor, and/or an aspartyl protease inhibitor. In some embodiments, the inhibitor is selected from the group of: Roche complete EDTA-free protease inhibitor cocktail (Sigma), 4(2-Aminoethyl)benzenesulfonyl Fluoride (MP Biomedical s), Pepstatin A (MP Biomedical s), GM6001 (EMD Millipore) and E-64 (Sigma). Additionally or alternatively, the inhibitor is water-solubilized and/or DMSO-solubilized, such as water-solubilized 4(2-Aminoethyl)benzenesulfonyl Fluoride (AEBSF, MP Biomedicals), water-solubilized E-64 (Sigma), DMSO-solubilized GM6001 (EMD Millipore), and DMSO-solubilized Pepstatin A (MP Biomedicals). In some embodiments, the inhibitor is one or more or all of those in Roche cOmplete EDTA-free protease inhibitor cocktail (Sigma). Additionally or alternatively, the inhibitor may comprise or consist essentially of, or yet further consist of one or more of a naturally occurring inhibitor, such as SerpinA1 and SerpinA3. Additionally or alternatively, the inhibitor reduces and/or inhibits one or more of the following protease activities: a serine-type endopeptidase activity, a serine-type peptidase activity, a metalloendopeptidase activity, a metal ion binding activity, a hydrolase activity, a dipeptidyl-peptidase activity, a serine-type aminopeptidase activity, a zinc ion binding activity, an aminopeptidase activity, and/or a metallopeptidase activity. More examples of inhibitor can be found at, e.g., www.drugs.com/drug-class/protease-inhibitors.html, go.drugbank.com/unearth/q?utf8=%E2%9C%93&searcher=drugs&query=Protease+inhibitor, and www.sigmaaldrich.com/life-science/biochemicals/biochemical-products.html?TablePage=15649049.

In yet a further aspect, the composition further comprises a conventional treatment, such as a non-specific immunosuppressive agent. In some embodiments, the conventional treatment is selected from one or more of the following: 5-ASA (also referred to as 5 aminosalicylate therapy, widely used in the management of mild to moderate IBD), an IS (for example, azathioprine, 6-mercaptopurine, methotrexate, cyclosporine A, tacrolimus), a biologic (for example, a TNF antagonist, Vedolizumab (n=3), and/or tofacitinib), a seroid or a thiopurine. In some embodiments, the compositions further comprise a non-specific immunosuppressive agent targeting the host, e.g., steroids, thiopurines, and/or biologics.

In one aspect that is optionally be combined with one or more of other embodiment(s) and/or aspect(s) as disclosed herein, a composition as disclosed herein further comprises one or more of a carrier (optionally a pharmaceutically acceptable carrier), stabilizer, preservative, and/or a biocompatible scaffold.

Non-limiting examples pharmaceutically acceptable carriers include diluents, excipients or carriers that may be used in the compositions of the disclosure. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Non-limiting examples of biocompatible scaffolds, include a scaffold or matrix with the ability to upon administration to deliver the compositions to a subject or an environment to be treated.

The compositions can be formulated or processed for ease of administration, storage and application, e.g., frozen, lyophilized, suspended (suspension formulation) or powdered; and processed as a suppository, tablet, solution, suspensions, pills, capsules, sustained release formulation.

In some embodiments, the composition is formulated or processed for use in a subject. In one aspect, the subject is at risk of having a disease as disclosed herein and/or diagnosed with a disease. Additionally or alternatively, the subject comprises one or more of the protease(s) as disclosed herein, the protease target(s) as disclosed herein, or fragment(s) of each thereof in a sample isolated from the subject. In some embodiments, the sample is a fecal sample. In some embodiments, the subject comprises a higher abundance (such as higher in one or more of: absolute total number, relative total number, and/or number of different peptides) of one or more of the protease(s) as disclosed herein, the protease target(s) as disclosed herein, or fragment(s) of each thereof. In some embodiments, the relative number is calculated as abundance of the protease(s) as disclosed herein, the protease target(s) as disclosed herein, or fragment(s) of each thereof of Bacteroides vulgatus over abundance of the protease(s) as disclosed herein, the protease target(s) as disclosed herein, or fragment(s) of each thereof of Bacteroides theta. In some embodiments, the subject comprises (such as has and/or expresses and/or is detected with) one or more of protein(s) as identified in Table 7, optionally in a sample and further optionally in exosomes of a sample, such as a fecal sample and/or a fecal exosome. In some embodiments, the subject has been treated with a conventional treatment. In further embodiments, no disease remission and/or recovery was observed in the subject treated with a conventional treatment (i.e., the subject is resistant to a conventional treatment.

The compositions are formulated for in vivo or ex vivo use. For use in vivo, the compositions are formulated for administration locally or systemically, such as orally, enterally, rectally, urogenitally, vaginally, nasally (inhalation), intravenously or intramuscularly (injectable), topically, as a suppository, as a spray (aerosol administration), dry application by admixing in the soil, as a solute (for admixing with an aqueous environment). In one aspect, they are formulated in a dosage form. Suitable dosage forms include, but are not limited to a suppository, a powder, a liquid, a capsule, a chewable tablet, a swallowable tablet, a buccal tablet, a troche, a lozenge, a soft chew, a solution, a suspension, a spray, a tincture, a decoction, an infusion, and combinations thereof. For in vitro use, the compositions are formulated for use in assays to test efficacy and combination therapies, for example. As is apparent to the skilled artisan, the composition can be combined with the supernatant, or solution.

### Applications and Uses

In general, the compositions of this disclosure find use in therapeutic, agricultural and industrial microbial support, the components of the compositions and the carriers and additional agents are selected for the specified use. In one aspect, the composition is for the treatment of a farm animal or pet. In another aspect, the composition is selected for the treatment of human patients, e.g., adults, juveniles and fetus in utero.

In one aspect, the compositions provide one or more of supporting anti-bacterial immunity, enhancing or supporting the gastrointestinal barrier, or antagonizing disease-related bacterial infections. In another aspect, the compositions prevents pathogen colonization and/or limits excessive inflammatory responses by down-regulating cytokine and chemokine production. In yet another aspect, the compositions as provided herein are useful in preventing and/or treating a disease as disclosed herein.

In one aspect, the compositions are useful for the treatment of a mammal such as a human; simians, murines, such as, rats, mice, chinchilla, canine, such as dogs, leporids, such as rabbits, livestock, sport animals and pets. In another aspect, they are useful to treat agricultural crops such as corn, wheat, soybeans, and potatoes; domestic garden plants such as tomatoes, peppers, spinach, and beans. In a yet further aspect, they are useful for the treatment of contaminated water or soil, machinery and manmade structures.

The indications and uses vary with the environment. The compositions can be used in the treatment or prevention of a disease, e.g., psychological disorders, such as depression or anxiety, enteric infectious disease, infection-induced colitis, traveler's diarrhea, inflammatory bowel disease (IBD), Crohn's disease (CD), colitis, ulcerative colitis (UC), colorectal cancer, diarrheal illness, vaginosis, wound, burns, psoriasis, dermatitis, tooth decay, periodontitis, sinusitis, or any of chronic and/or recurrent disease that is caused by pathogenic bacteria displacing healthy bacteria to support anti-bacterial immunity, enhancing or supporting the gastrointestinal barrier, correcting or supporting dysbiotic gut flora (and even in the absence of diseases), disease or disorders involving intestinal dysmobility, enhancing or supporting the gastrointestinal mobility, or antagonizing disease-related bacterial infection; UC, CD, colitis or traveler's diarrhea, peritonitis, post-operative ileus, irritable bowel syndrome (IBS), inflammatory bowel disease, intestinal pseudo-obstruction, and/or constipation.

Thus, in one aspect, this disclosure provides method for treating or preventing a disease or disorder as disclosed herein in a subject in needed thereof. In some embodiments, the disease or disorder is suitably related to an aberrant immune response directed toward the gut microbiota in a subject in need thereof. The method comprises or consists essentially of, or yet further consists of administering to the subject, for example, an effective amount of, the composition as disclosed herein, having the components selected for the particular therapy. Non-limiting examples of diseases include psychological disorders, such as depression or anxiety, enteric infectious disease, infection-induced colitis, traveler's diarrhea, inflammatory bowel disease (IBD), UC, CD, colitis, colorectal cancer, diarrheal illness, vaginosis, wound, burns, psoriasis, dermatitis, tooth decay, periodontitis, sinusitis, or any of chronic and/or recurrent disease that is caused by pathogenic bacteria displacing healthy bacteria, and to support anti-bacterial immunity, enhancing or supporting the gastrointestinal barrier, correcting or supporting dysbiotic gut flora (and even in the absence of diseases), disease or disorders involving intestinal dysmobility, enhancing or supporting the gastrointestinal mobility, or antagonizing disease-related bacterial infection; vaginosis; peritonitis, post-operative ileus, irritable bowel syndrome (IBS), colorectal cancer, intestinal pseudo-obstruction, and/or constipation. Additionally, the compositions are useful to promote health and/or to maintain gut homeostasis.

In some embodiments, a disease as disclosed herein is resistant to a conventional treatment. In some embodiments, the disease is ulcerative colitis (UC). In some embodiments, the disease is Crohn's disease (CD). In further embodiments, the diseases is one or more of: colonic CD, ileal CD or ileocolonic CD. In some embodiments, the method comprises or consists essentially of, or yet further consists of administering to a subject in need thereof, for example an effective amount of, a composition as disclosed herein.

In one aspect, provided is a method for one or more of: supporting anti-bacterial immunity, correcting dysbiosis, enhancing or supporting the gastrointestinal barrier, supporting or enhancing gastrointestinal motility, localized release of antibiotic compositions, or antagonizing disease-related bacterial infections; or treating one or more of: inflammatory bowel disease (IBD), irritable bowel syndrome (IBS), colorectal cancer, chronic inflammation of the colon, colectomy, dysbiosis, colitis, ulcerative colitis (UC), Crohn's disease (CD), enteric infectious disease, diarrheal illness, vaginosis, wound, burns, psoriasis, dermatitis, tooth decay, periodontitis, sinusitis, infection-induced colitis, traveler's diarrhea, psychological stress, psychological disorders, or any of chronic or recurrent disease that is caused by pathogenic bacteria displacing healthy bacteria in the gut or digestive tract. The method comprises or consists essentially of, or yet further consists of administering to a subject in need thereof, for example an effective amount of, a protease inhibitor that targets a protease expressed by a pathogenic bacterium. In some embodiments, the method comprises or consists essentially of, or yet further consists of administering to a subject in need thereof, for example an effective amount of, a composition as disclosed herein.

In one aspect, provided is a method for preventing one or more of: inflammatory bowel disease (IBD), irritable bowel syndrome (IBS), colorectal cancer, chronic inflammation of the colon, colectomy, dysbiosis, colitis, ulcerative colitis (UC), Crohn's disease (CD), enteric infectious disease, diarrheal illness, vaginosis, wound, burns, psoriasis, dermatitis, tooth decay, periodontitis, sinusitis, infection-induced colitis, traveler's diarrhea, psychological stress, psychological disorders, or any of chronic or recurrent disease that is caused by pathogenic bacteria displacing healthy bacteria in the gut or digestive tract. The method comprises or consists essentially of, or yet further consists of administering to a subject in need thereof, for example an effective amount of, a protease inhibitor that targets a protease expressed by a pathogenic bacterium. In some embodiments, the method comprises or consists essentially of, or yet further consists of administering to a subject in need thereof, for example an effective amount of, a composition as disclosed herein.

In some embodiments, the pathological bacteria comprises or consists essentially of, or yet further consists of a Bacteroides bacterium. In further embodiments, the Bacteroides is one or more of a Bacteroides identified in FIG. 7d. In yet further embodiments, the Bacteroides is one or more of a Bacteroides vulgatus, Bacteroides dorei, Bacteroides theta or Bacteroides uniformis.

In some embodiments, the protease is selected from one or more of a serine-protease, a metalloproteinase, an aspartyl protease and a cysteine-protease. In some embodiments, the protease is selected from any one of FIGS. 7e, 11e, 11f, 11g, and/or 18a-18e and/or those identified in Table 4.

In some embodiments, the inhibitor is selected from one or more of: AEBSF, E-64, GM6001, and Pepstatin A.

In some embodiments, a method as disclosed herein further comprises administering to the subject, for example an effective amount of, a conventional treatment, such as a non-specific immunosuppressive agent. In further embodiments, the non-specific immunosuppressive agent is selected from a seroid or a thiopurine.

In some embodiments, the subject has (such as comprises, displays, shows, and/or expresses) one or more of, optionally in a sample as disclosed herein: a high level of a protease as disclosed herein, such as one expressed by the pathogenic bacteria; a high activity of a protease as disclosed herein, such as one expressed by the pathogenic bacteria; a high level of a target or a fragment thereof and/or a peptide; an altered expression (such as increased expression level) of one or more of protein(s) as identified in Table 7, optionally in a sample and further optionally in exosomes of a sample, such as a fecal sample and/or a fecal exosome; an altered expression of a tight junction protein, such as ZO-1 and/or occludin; a decrease in epithelial cell circularity; an increased permeability of an epithelial cell layer; or resistance to a conventional treatment. In some embodiments, the conventional treatment is selected from a non-specific immunosuppressive agent. In some embodiments, the conventional treatment is selected from one or more of the following: 5-ASA (also referred to as 5 aminosalicylate therapy, widely used in the management of mild to moderate IBD), an IS (for example, azathioprine, 6-mercaptopurine, methotrexate, cyclosporine A, tacrolimus), a biologic (for example, a TNF antagonist, Vedolizumab (n=3), and/or tofacitinib), a seroid or a thiopurine. In some embodiments, the target or a fragment thereof is a peptide, optionally selected from a dipeptide or an oligopeptide. In some embodiments, the peptide is selected from a target of the protease or a fragment thereof. Additionally or alternatively, the target is selected from one or more of a collagen, a mucin or a peptide as disclosed herein. In some embodiments, the reference relating to the relative terms here is the corresponding level, activity, expression, circularly, permeability and/or resistance of a healthy subject or a subject free of the disease.

In some embodiments, the subject is an animal or mammal. In further embodiments, the mammal is a human patient.

In some embodiments, the protease inhibitor is administered locally or systemically. In further embodiments, the protease inhibitor is administered orally to the subject.

In some embodiments, the protease inhibitor is formulated in a pharmaceutical acceptable carrier. Additionally or alternatively, the protease inhibitor is formulated in a dosage form selected from the group consisting of: suppository, within a biocompatible scaffold, powder, liquid, capsule, chewable tablet, swallowable tablet, buccal tablet, troche, lozenge, soft chew, solution, suspension, spray, tincture, decoction, infusion, and combinations thereof.

In some embodiments, a method as disclosed herein further comprises assaying a sample isolated from the subject for one or more of the following: a level of a protease, such as one expressed by the pathogenic bacteria; a protease activity; a level of a target or a fragment thereof, and/or a peptide; a level of one or more of protein(s) as identified in Table 7, optionally in a sample and further optionally in exosomes of a sample, such as a fecal sample and/or a fecal exosome; expression of a tight junction protein; epithelial cell circularity; or permeability of an epithelial cell layer. In some embodiments, the target or a fragment thereof is a peptide, optionally selected from a dipeptide or an oligopeptide. In some embodiments, the peptide is selected from a target of the protease or a fragment thereof. Additionally or alternatively, the target is selected from one or more of a collagen, a mucin or a peptide as disclosed herein.

In some embodiments, one or more of the treatment effects can be evaluated by one of skill in the art, such as utilizing colitis score, crypt length, normal colon length, inflammation status (for example shown by spleen size), endoscopic disease activity assessment (e.g., those using partial Mayo, the Mayo endoscopic sub-score, the Ulcerative Colitis Endoscopic Index of Severity (UCEIS), the Simple Endoscopic Score for Crohn Disease (SES-CD), the Crohn's Disease Activity Index (CDAI), blinded histology, biomarkers, and/or the Geboes score). In some embodiments, one or more of the following indicates a successful treatment: decreased score, improved assessment, decreased crypt length, increased normal colon length, reduced inflammation (such as, less swollen spleen), compared to those prior to the treatment.

The protease inhibitor composition can be administered alone or in combination with one or more non-specific immunosuppressive agents targeting the host, e.g., steroids, thiopurines, and/or biologics. They can be administered concurrently or sequentially.

The compositions can be administered orally, vaginally, topically, by inhalation, intravenously, intramuscularly, or by suppository. They can be administered in any suitable formulation. Non-limiting examples of route of administration is local or systemical, including oral administration, enteral administration, rectal administration, urogenital (such as vaginal) administration, nasal administration (inhalation), injection (such as intravenous or intramuscular), topical application, by suppository, as a spray (aerosol administration), dry application, or as a solute (for admixing with an aqueous environment).

For the treatment or prevention of plant disease or in agricultural settings, the composition are useful for the treatment of desiccation, nutrient starvation, nutrient depletion, bacterial pathogen infection, invertebrate antagonism, pollution; severe weather, physical stress, hypoxia, soil acidification. Thus, this disclosure also provides methods for treating a plant, by administering to the plant directly or in its environment, a composition as disclosed herein. The dosage and components of the composition will vary with the plant and purpose of the treatment.

The compositions can be administered at about 6, 12, 18, 24, 36, 48, and 72 hours, or can be administered in a single dose. In one aspect, the composition is administered by spraying the plant or by irrigating the plant or admixing the composition with water applying to the plant or its environment. It can by sprayed onto the plant or the soil surrounding the plant, applied dry into the soil surface surrounding the plant, adding the compositions to the irrigation or watering system, or mixing the composition with the soil prior to seeding.

Thus, this disclosure also provides methods to deliver a composition and/or treat or prevent a disease or condition, and/or treat an environment (soil, plant, water, or surface) by contacting the surface or delivering an effective amount of the composition as disclosed herein.

The compositions can be formulated or processed for ease of administration, storage and application, e.g., frozen, lyophilized, suspended (suspension formulation) or powdered, and processed for use in industrial applications, e.g., for the treatment of contaminated water or soil, machinery, and manmade structures, e.g., bioreactor, biopile, bio-venting, land-farming, filter surface, permeable reactive barrier, in situ administration via wet or dry application to water or soil.

The amount and components of the composition will vary with the purpose of the treatment.

One can determine if the treatment has been successful by monitoring for a reduction in disease symptoms and/or by assaying or assaying for the presence of peptide fragments in samples isolated from treated patients. Assaying for the presence of microbial peptide fragments as disclosed herein is a useful pre-treatment diagnostic test that can be combined with the therapeutic and prophylactic uses disclosed herein.

### Nutritional Supplements

The disclosed compositions also are useful as nutritional supplements to promote general health and well-being and maintain gut health and/or homeostasis. Thus, in one aspect, this disclosure also provides a method for promoting health and/or maintaining gut homeostasis in a subject in need thereof, the method comprising, or alternatively consisting essentially of, or yet further consisting of, administering to the subject an effective amount of a composition as described herein, alone or in combination with a non-specific immunosuppressive agent targeting the host. One of skill in the art can determine if better general health has been achieved, as well as gut homeostatis, by determining if gut discomfort has been reduced or alleviated.

### Pharmaceutical Formulations

The composition can be formulated as a frozen composition, e.g., flash frozen, dried or lyophilized for storage and/or transport. In addition, the composition can administered alone or in combination with a carrier, such as a pharmaceutically acceptable carrier or a biocompatible scaffold. Compositions of the disclosure can be conventionally administered rectally as a suppository, parenterally, by injection, for example, intravenously, subcutaneously, or intramuscularly. Non-limiting examples of route of administration is local or systemical, including oral administration, enteral administration, rectal administration, urogenital (such as vaginal) administration, nasal administration (inhalation), injection (such as intravenous or intramuscular), topical application, by suppository, as a spray (aerosol administration), dry application, or as a solute (for admixing with an aqueous environment). Additional formulations which are suitable for other modes of administration include oral formulations. Oral formulations include such normally employed excipients such as, for example, pharmaceutical grades of mannitol, lactose, starch, magnesium stearate, sodium saccharine, cellulose, magnesium carbonate and the like. These compositions take the form of solutions, suppositories, suspensions, tablets, pills, capsules, sustained release formulations or powders and contain about 10% to about 95% of active ingredient, preferably about 25% to about 70%.

Typically, compositions are administered in a manner compatible with the dosage formulation, and in such amount as will be therapeutically effective for the disease or condition by treated. The quantity to be administered depends on the subject to be treated. Precise amounts of the composition to be administered depend on the judgment of the practitioner. Suitable regimes for initial administration and boosters are also variable, but are typified by an initial administration followed by subsequent administrations.

In many instances, it will be desirable to have multiple administrations of the compositions about, at most about or at least about 3, 4, 5, 6, 7, 8, 9, 10 days or more. The administrations will normally range from 2 day to twelve week intervals, more usually from one to two week intervals. Periodic boosters at intervals of 0.5-5 years, usually two years, can be desirable to maintain the condition of the immune system.

In some embodiments, additional pharmaceutical compositions are administered to a subject to support or augment the compositions as described herein. Different aspects of the present disclosure involve administering an effective amount of the composition to a subject. Additionally, such compositions can be administered in combination with modifiers of the immune system. Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated.

The carrier can be a solvent or dispersion medium containing, for example, water, ethanol, polyol (for example, glycerol, propylene glycol, and liquid poly(ethylene glycol), and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of undesirable microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

An effective amount of therapeutic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

### Processes for Preparing Compositions

This disclosure also provides a method for preparing a composition as described herein, comprising, or alternatively consisting essentially of, or yet further consists of, the steps of admixing, contacting or culturing one or more protease inhibitor with a suitable carrier. Additional components, as disclosed herein, can be further admixed.

### Kits

In some embodiments, a kit containing one or more compositions as described herein is provided. The kit comprises, or alternatively consists essentially of, or yet further consists of, a composition as described above, and instructions for use.

In some embodiments, the kit comprises or consists essentially of, or yet further consists of one or more of a protease inhibitor (such as an effective amount of a protease inhibitor). In some embodiments, the kit comprises or consists essentially of, or yet further consists of a composition as disclosed herein. In some embodiments, the inhibitor targets a protease expressed by a pathogenic bacterium. In some embodiments, the kit comprises or consists essentially of, or yet further consists of one or more of reagent(s) and/or buffer(s) for detecting expression and/or activity level(s) of a protease as disclosed herein, expression of one or more of protein(s) as identified in Table 7, optionally in a sample and further optionally in exosomes of a sample, such as a fecal sample and/or a fecal exosome sample, expression level of a peptide as disclosed herein, expression (such as level and/or pattern) of a tight junction protein, epithelial cell circularity; permeability of an epithelial cell layer. Such reagents can include a target of the protease conjugated to a detectable label and/or antibodies specifically recognizing and binding to the protease and/or the one or more of protein(s) as identified in Table 7. In further embodiments, the antibodies can be conjugated to a detectable label. Additionally or alternatively, the kit is for use in a method as disclosed herein. In some embodiments, the method further comprises a non-specific immunosuppressive agent (such as an effective amount of the non-specific immunosuppressive agent(s)). Additionally or alternatively, the method further comprises instructions for use.

### Diagnostic Methods

This disclosure also provides a method to diagnose and stratify a patient for inflammatory bowel disease (IBD), UC and CD by analyzing a sample from the patient for the presence of Bacteroides or a Bacteroides protease, and in particular serine proteases from Bacteroides vulgatus, wherein the presence is an indication that the patient is suffering from IBD, UC, or CD. These proteases and their relationship to disease activity are further detailed in the Experimental Examples. In a further aspect, patients identified as having IBD, UC or CD can be treated with the compositions as provided herein. Thus, in one aspect, also provided are methods for treating patients identified as having the presence of a Bacteroides or a Bacteroides protease with a composition as described herein.

In some embodiments, provided is a method to identify a subject for protease therapy. In some embodiments, provided is a method to identify a subject having a high risk of developing a disease as disclosed herein. In some embodiments, provided is a method to identify a subject having a high risk of developing a disease as disclosed herein with high severity. In some embodiments, provided is a method to identify a subject having a disease as disclosed herein. In some embodiments, provided is a method to identify a subject diagnosed with a disease as disclosed herein and having a high risk of developing the disease to a severe stage. In some embodiments, provided is a method to identify a subject diagnosed with a disease having a high risk of being resistant to a conventional treatment.

The method comprises or consists essentially of, or yet further consists of assaying a sample isolated from the subject for one or more of the following: a level of protease as disclosed herein, such as one expressed by the pathogenic bacteria; a protease activity; a level of a target or a fragment thereof, and/or a peptide; expression of a tight junction protein; epithelial cell circularity; permeability of an epithelial cell layer; presence and/or abundance of protein(s) as identified in Table 7, optionally in a sample and further optionally in exosomes of a sample, such as a fecal sample and/or a fecal exosome sample. In some embodiments, one or more of the following identifies the patient for protease therapy: higher than normal levels of any one or more of the protease, the protease activity, or the peptides; an altered expression of a tight junction protein; a decrease in epithelial cell circularity; an increased permeability of an epithelial cell layer; presence of one or more of protein(s) as identified in Table 7, optionally in a sample and further optionally in exosomes of a sample, such as a fecal sample and/or a fecal exosome. In some embodiments, the reference for the relative terms as used herein is those of a healthy subject and/or a subject free of the disease or disorder. In some embodiment, the method further comprises administering to the subject, for example an effective amount of, a protease inhibitor to the identified subject. Additionally or alternatively, the method further comprises comprising to the subject administering, for example an effective amount of, a conventional treatment to the subject. In some embodiments, the conventional treatment is a non-specific immunosuppressive agent.

In some embodiments which can optionally be combined with any other embodiments and/or any aspect as described herein, a sample is isolated from a subject. In further embodiments, such collected sample can be further processed to generate a sample for use. Non-limiting examples of such processing include one or more of: purification, isolation, concentration and/or removal of certain components. In some embodiments, the sample is a serum sample. In some embodiments, the sample is a fecal sample. In further embodiments, the fecal sample is collected and frozen immediately. In some embodiments, the sample is a mucosal biopsy, optionally from one or more of colon, small intestine, rectum and/or other parts of the GI.

The following examples are intended to illustrate, and not limit the disclosure.

### EXPERIMENTAL EXAMPLES

### Experimental Example No. 1—Meta-Omics Reveals Microbiome Driven Proteolysis as a Contributing Factor to Severity of Ulcerative Colitis Disease Activity

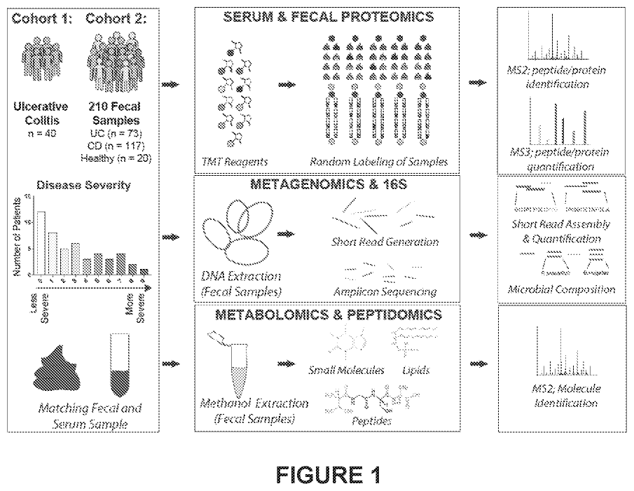

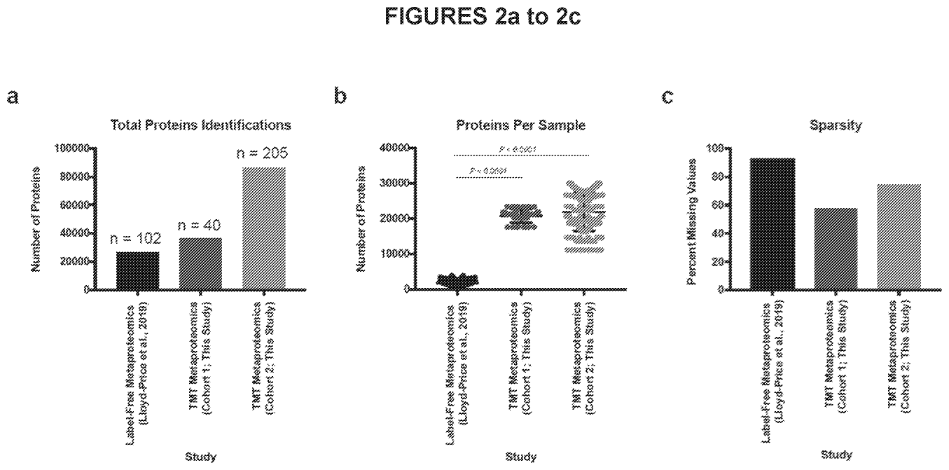

In the study herein, Applicant selected patient samples from a convenience biobank at a single academic IBD center (UC San Diego) who underwent extensive phenotyping with clinical disease activity indices and blinded assessments of endoscopic and histologic severity¹²⁻¹⁴ (Table 1). Applicant's study design included an initial discovery cohort of 40 UC patients and a separately collected, more complex cohort of 210 patient samples including 53 UC and 101 Crohn's disease patients (CD; roughly split by ileal, ileocolonic, and colonic subtypes), and 20 healthy volunteers. Individual patient matched serum and fecal samples were subset for shotgun metagenomic, 16S rRNA gene amplicon sequencing, metabolomic, serum proteomic, metapeptidomics, and metaproteomic analyses (FIG. 1). A previously established integrated metagenomic-metaproteomic approach of shared database assembly and quantification were used for direct comparisons between data types¹⁵. Notably, application of Applicant's multiplexing metaproteomic methods provided increased depth and a greater than 10-fold increase in proteins quantified per sample in comparison to previous metaproteomic methodology as exemplified in the Human Microbiome Project's IBD multi-omics database⁶ (FIG. 2^{6,16,17}).

Others in the field have already tried to combine these omics methods together (an example is seen in a Nature paper⁶) and they did not make the discovery that Applicant has made here despite having all the same omics approaches available in a similar cohort of patients with the same disease type (UC and CD). Applicant's novel approach including combined and analyzed the data, as well as the isolation of specific strains of bacteria, and confirmation studies to confirm initial observations all lead to the discoveries and applications of those discoveries as disclosed herein. Also, it is over abundance of proteases being detected in stool which means Applicant was seeing more of the protease in the stool than one of skill in the art would have expected based on the number of bacteria present, and these bacteria were therefore producing more than anticipated amount of its protease(s).

TABLE 1
Patient characteristics

| Baseline Characteristic | UC patients (n = 40) | |
|---|---|---|
| Age, median (IQR) | 38.5 | (28.5-52.5) |
| Female gender, n (%) | 11 | (28%) |
| Caucasian race, n (%) | 30 | (75%) |
| Smoker, n (%) | 3 | (7.5%) |
| Height, median (IQR) | 176.5 | (166.4-184.8) |
| Disease duration. median (IQR) | 8 | (4.5-19) |
| Historic extent pancolitis, n (%) | 18 | (45%) |
| Prior 5-ASA exposure, n (%) | 36 | (90%) |
| Current 5-ASA exposure, n (%) | 20 | (50%) |
| Prior steroid exposure, n (%) | 29 | (73%) |
| Current steroid exposure, n (%) | 6 | (15%) |
| Prior IS exposure, n (%) | 16 | (40%) |
| Current IS exposure, n (%) | 7 | (18%) |
| Biologic exposure*, n (%) | 19 | (48%) |
| Partial Mayo score, median (IQR) | 3 | (1-5.75) |

All baseline characteristics are at time of sample collection. Endoscopic scoring was done blinded to clinical data or other available biomarker data, and histologic scoring was done blinded to clinical, biomarker, and endoscopic data. Endoscopic scoring was performed by a physician with expertise and advanced training in UC, and histologic scoring was performed by a pathologist with expertise and advanced training in GI pathology.

Five patients were early (≤2 years) in the disease course, and 6 patients had disease limited to the rectum. Clinical remission (partial Mayo Score of ≤2) was observed in 18 patients (45%) at the time of sample collection. Of the 26 patients with no rectal bleeding, 14 had persistent diarrhea (54%) not related to infectious or alternative etiologies. Endoscopic disease activity was well distributed across the cohort with Mayo endoscopic subscores of 0, 1, 2, and 3 being observed in 11 (28%), 10 (25%), 10 (25%), and 9 (22%) patients, respectively. Histologic remission was observed in 10 (25%) patients, and 7 patients had both endoscopic (Mayo endoscopic sub-score of 0) and histologic remission. *all patients with prior biologic exposure were on a biologic at time of sample collection. 5-ASA-5 aminosalicylate therapy. IS: azathioprine, 6-mercaptopurine, methotrexate. Biologics included TNF antagonists (n=15), Vedolizumab (n=3), and 1 patient on tofacitinib.

Meta-Omic Associations with UC Severity

TABLE 2
Association of clinical 19 variables to beta-diversity

| | | | Microbial | | 16S | Metagenomics | 16S | Metagenomics |
|---|---|---|---|---|---|---|---|---|
| | Serum | | Proteins | | Bray- | Bray- | Unweighted | Unweighted |
| | Proteomics | Metaproteomics | Only | Metabolomics | Curtis | Curtis | UniFrac | UniFrac |
| Calprotectin | 0.019*, | 0.001***, | 0.003**, | 0.001***, | 0.008**, | 0.019*, | 0.003**, | 0.001***, |
| | 2.405 | 4.818 | 2.026 | 3.347 | 1.855 | 1.509 | 2.102 | 7.106 |
| Partial | 0.014*, | 0.001***, | 0.044*, | 0.001***, | 0.013*, | 0.101, | 0.021*, | 0.001***, |
| Mayo | 2.509 | 3.156 | 1.506 | 2.559 | 1.609 | 1.212 | 1.695 | 4.587 |
| Stool | 0.028*, | 0.001***, | 0.024*, | 0.001***, | 0.005**, | 0.054, | 0.004**, | 0.001***, |
| Frequency | 2.328 | 3.175 | 1.583 | 2.377 | 1.952 | 1.31 | 2.084 | 3.73 |
| Mayo | 0.008**, | 0.003**, | 0.126, | 0.002**, | 0.24, | 0.279, | 0.132, | 0.001***, |
| Endoscopic | 2.392 | 2.25 | 1.251 | 2.005 | 1.16 | 1.061 | 1.254 | 3.322 |
| Score | | | | | | | | |
| UCEIS | 0.024*, | 0.001***, | 0.054, | 0.001***, | 0.112, | 0.141, | 0.062, | 0.001***, |
| Endoscopic | 2.237 | 3.022 | 1.462 | 2.534 | 1.292 | 1.169 | 1.4 | 4.678 |
| Score | | | | | | | | |
| Rectal | 0.222, | 0.002**, | 0.099, | 0.002**, | 0.44*, | 0.078, | 0.051, | 0.001***, |
| Bleeding | 1.225 | 2.271 | 1.23 | 2.392 | 1.499 | 1.27 | 1.482 | 3.913 |
| PGA | 0.029*, | 0.018*, | 0.27, | 0.029*, | 0.524, | 0.528, | 0.345, | 0.002**, |
| | 2.1625 | 1.818 | 1.094 | 1.589 | 0.96 | 0.962 | 1.03 | 2.641 |
| C-reactive | 0.032* | 0.022*, | 0.132, | 0.015*, | 0.344, | 0.127, | 0.299, | 0.132, |
| Protein | 2.374 | 1.918 | 1.316 | 1.605 | 1.08 | 1.282 | 1.112 | 1.507 |
| Current | 0.927, | 0.055, | 0.329, | 0.351, | 0.696, | 0.489, | 0.573, | 0.183, |
| Steroids | 0.605 | 1.517 | 1.074 | 1.059 | 0.89 | 0.969 | 0.941 | 1.369 |
| Collection | 0.347, | 0.243, | 0.486, | 0.726, | 0.596, | 0.437, | 0.181, | 0.212, |
| Timestamp | 1.13 | 1.121 | 1.007 | 0.939 | 0.983 | 1.015 | 1.12 | 1.236 |
| ASA | 0.306, | 0.598, | 0.924, | 0.297, | 0.684, | 0.28, | 0.212, | 0.114, |
| Exposure | 1.063 | 0.884 | 0.924 | 1.129 | 0.862 | 1.056 | 1.13 | 1.474 |
| IM exposure | 0.293, | 0.117, | 0.099, | 0.07, | 0.326, | 0.045*, | 0.026*, | 0.112, |
| | 1.133 | 1.31 | 1.335 | 1.418 | 1.085 | 1.372 | 1.635 | 1.458 |
| Sex | 0.646, | 0.107, | 0.207, | 0.455, | 0.101, | 0.139, | 0.128, | 0.336, |
| | 0.778 | 1.358 | 1.152 | 0.985 | 1.327 | 1.164 | 1.223 | 1.09 |
| Biologic | 0.312, | 0.058, | 0.092, | 0.015*, | 0.006**, | 0.025*, | 0.198, | 0.191, |
| exposure | 1.06 | 1.238 | 1.18 | 1.216 | 1.305 | 1.215 | 1.079 | 1.174 |
| type | | | | | | | | |
| Current | 0.411, | 0.408, | 0.287, | 0.004**, | 0.288, | 0.727, | 0.103, | 0.475, |
| 5ASA | 0.964 | 1.019 | 1.103 | 2.115 | 1.112 | 0.894 | 1.26 | 0.946 |
| Current | 0.167, | 0.2, | 0.442, | 0.019*, | 0.084, | 0.064, | 0.092, | 0.186, |
| Biologic | 1.33 | 1.196 | 0.995 | 1.686 | 1.376 | 1.311 | 1.368 | 1.323 |
| Disease | 0.408, | 0.392, | 0.507, | 0.608, | 0.623, | 0.655, | 0.832, | 0.277, |
| Duration | 1.002 | 1.024 | 0.954 | 0.885 | 0.904 | 0.908 | 0.799 | 1.129 |
| Biologic | 0.184, | 0.196, | 0.421, | 0.021*, | 0.072, | 0.054, | 0.059, | 0.162, |
| exposure | 1.326 | 1.196 | 0.995 | 1.686 | 1.376 | 1.311 | 1.079 | 1.323 |
| Height | 0.909, | 0.591, | 0.906, | 0.593, | 0.448, | 0.414, | 0.664, | 0.45, |
| | 0.55 | 0.902 | 0.74 | 0.91 | 1.027 | 0.994 | 0.885 | 0.959 |
| Experiment | NA, | 0.167, | 0.035*, | NA, | NA, | NA, | NA, | NA, |
| | NA | 1.15 | 1.266 | NA | NA | NA | NA | NA |
| Age | 0.005**, | 0.203, | 0.11, | 0.307, | 0.452, | 0.209, | 0.284, | 0.44, |
| Diagnosis | 2.676 | 1.181 | 1.289 | 1.106 | 0.995 | 1.119 | 1.06 | 0.963 |
| Endoscopy | 0.201, | 0.258, | 0.246, | 0.674, | 0.588, | 0.686, | 0.106, | 0.555, |
| Date | 1.217 | 1.104 | 1.095 | 0.951 | 0.972 | 0.956 | 1.14 | 0.986 |
| Age | 0.01**, | 0.234, | 0.108, | 0.113, | 0.448, | 0.581, | 0.532, | 0.38, |
| | 2.775 | 1.155 | 1.283 | 1.328 | 0.997 | 0.935 | 0.931 | 1.001 |
| Historic | 0.183, | 0.231, | 0.225, | 0.018*, | 0.254, | 0.055, | 0.148, | 0.116, |
| Extent | 1.243 | 1.128 | 1.113 | 1.486 | 1.103 | 1.219 | 1.157 | 1.332 |
| Steroid | 0.22, | 0.151, | 0.073, | 0.12, | 0.023*, | 0.035*, | 0.709, | 0.311, |
| Exposure | 1.305 | 1.244 | 1.35 | 1.319 | 1.606 | 1.384 | 0.873 | 1.074 |
| TMT Label | NA, | 0.267, | 0.137, | NA, | NA, | NA, | NA, | NA, |
| | NA | 1.062 | 1.113 | NA | NA | NA | NA | NA |
| Current | 0.495, | 0.364, | 0.267, | 0.437, | 0.068, | 0.051, | 0.646, | 0.535, |
| Biologic | 0.96 | l.049 | 1.084 | 1.013 | 1.211 | 1.199 | 0.956 | 0.953 |
| Type | | | | | | | | |
| Race | 0.695, | 0.608, | 0.413, | 0.495, | 0.563, | 0.818, | 0.369, | 0.789, |
| | 0.844 | 0.939 | 1.008 | 1.002 | 0.972 | 0.895 | 1.031 | 0.794 |
| IM type | 0.806, | 0.766, | 0.518, | 0.672, | 0.816, | 0.589, | 0.168, | 0.824, |
| | 0.801 | 0.891 | 0.981 | 0.945 | 0.894 | 0.958 | 1.106 | 0.781 |

Categorical variables tested through PERMANOVA, continuous variables 20 tested through Adonis. P-values and Pseudo-F values for each category are reported. Significance level is indicated according to p-value (*<0.05, **<0.01, ***<0.001). Testing was based on Bray-Curtis distances unless otherwise specified. Results shown here are from UC Cohort 1.

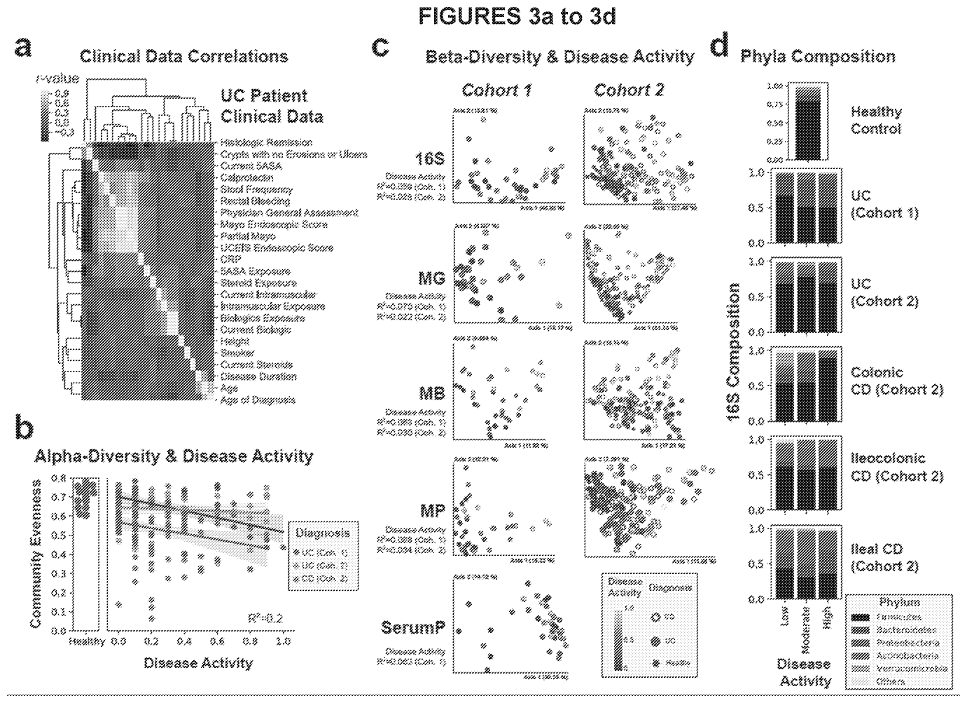

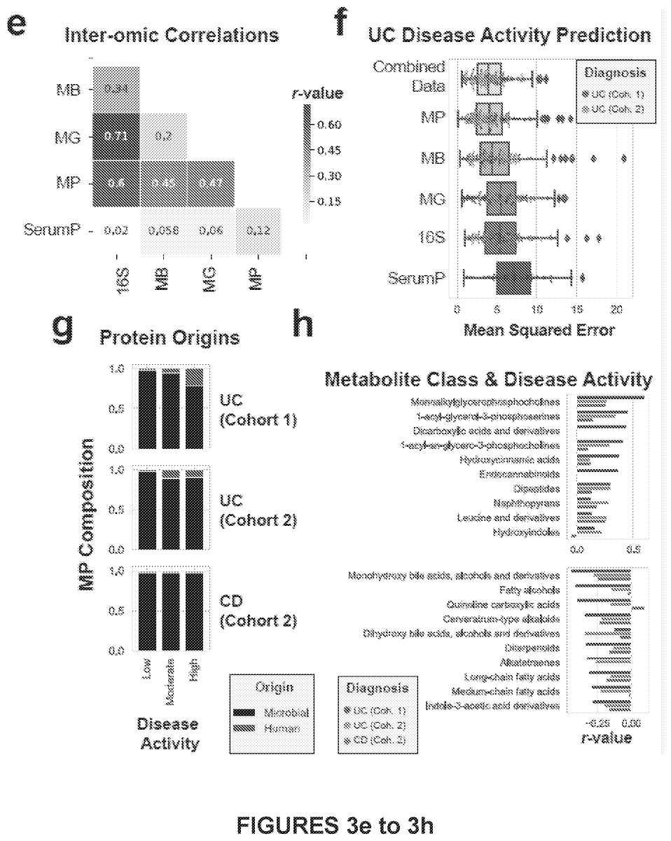

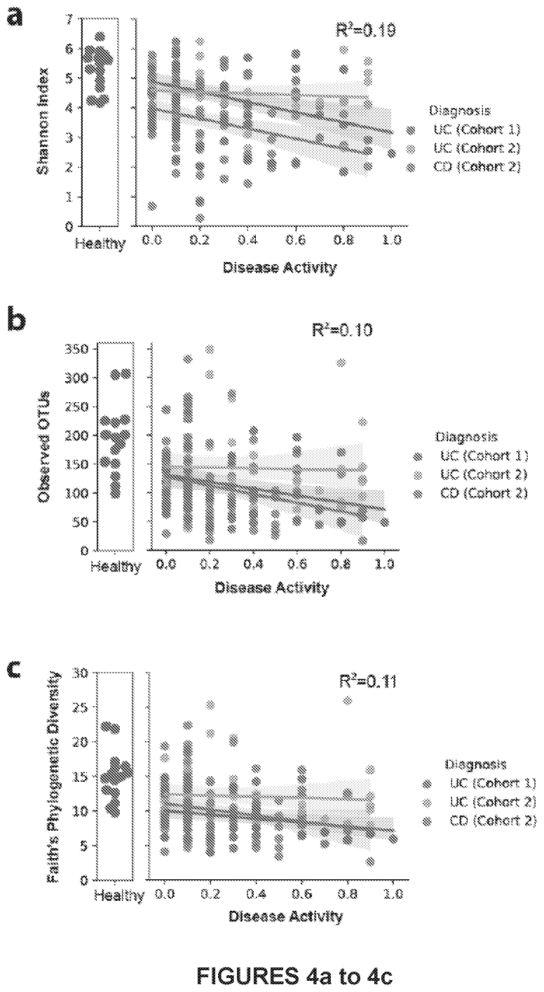

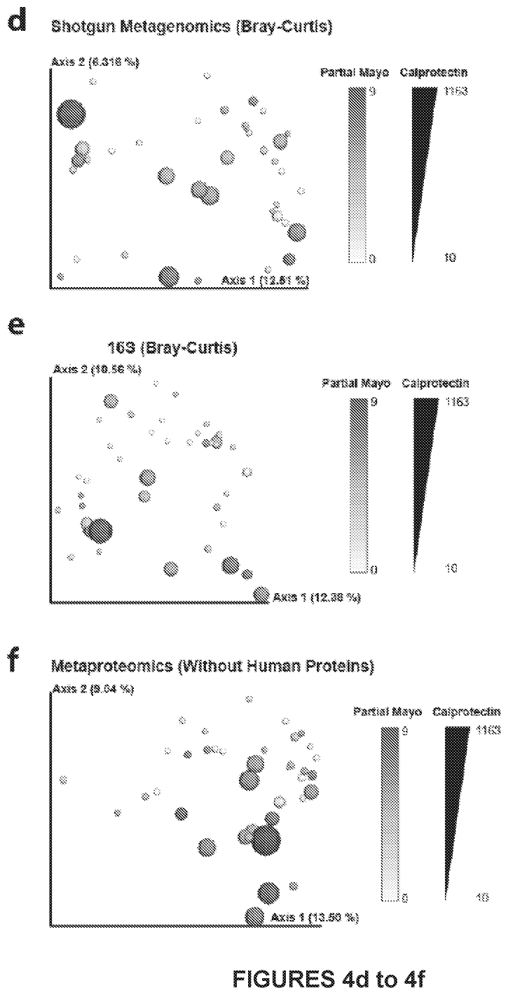

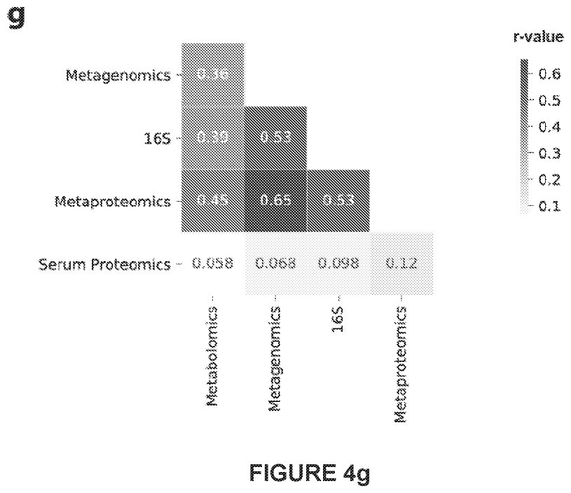

A range of endoscopic and clinical metrics were collected, with most of the measured clinical severity metrics showing a high degree of correlation (FIG. 3a). Given the overlapping signal of severity metrics, a representative symptom metric was chosen, partial Mayo for UC, alongside the Crohn's Disease Activity Index (CDAI) for CD, as the primary severity metric. Endoscopy and histology metrics for UC were subsequently used to confirm observations and associations. The severity metrics were significantly correlated with both alpha and beta-diversity metrics in all meta-omics collected (FIGS. 3b and 3c, Table 2, FIG. 4). The data suggests that diagnosis is not the most prominent influence on beta-diversity. CD subtypes and the two separately processed UC cohorts displayed unique microbiota compositions distinct from healthy controls (FIGS. 3d and 5). Comparing—omic data types, Applicant observed stronger correlations between the distributions of data in the fecal based-omics than serum proteome (FIGS. 3e and 4), and that combining all data types provided the strongest prediction of UC activity though closely followed by the metaproteome and metabolome (FIG. 3f). Unlike UC, an influential feature in CD patient microbiomes was the dominance of a member of the Enterobacteriaceae family (FIG. 6).

Table 3 shows activity list of bacteria identified in the samples. Features of most importance to predicting disease activity were listed and summarized but not shown herein. Feature importance values from the 100 random forest iterations predicting UC severity from cohort 1 (summarized in FIG. 3f) were summed and ranked by their total importance. Top features and annotation information is shown for each data type as well as the combined data classification. As used in the table below, k stands for kingdom; p is short for phylum; c stands for class; o stands for order; f is short for family; g is short for genus; and s stands for species.

k_Bacteria; p_[Thermi]; c_Deinococci; o_Deinococcales; f_Deinococcaceae;
g_Deinococcus; s_geothermalis
k_Bacteria; p_[Thermi]; c_Deinococci; o_Deinococcales; f_Trueperaceae;
g_Truepera; s_{—}
k_Bacteria; p_[Thermi]; c_Deinococci; o_Thermales; f_Thermaceae;
g_Meiothermus; s_{—}
k_Bacteria; p_[Thermi]; c_Deinococci; o_Thermales; f_Thermaceae; g_Thermus;
s_{—}
k_Bacteria; p_Actinobacteria; c_Actinobacteria; o_Actinomycetales;
f_Actinomycetaceae; g_; s_{—}
k_Bacteria; p_Actinobacteria; c_Actinobacteria; o_Actinomycetales;
f_Actinomycetaceae; g_Actinomyces; s_{—}
k_Bacteria; p_Actinobacteria; c_Actinobacteria; o_Actinomycetales;
f_Corynebacteriaceae; g_Corynebacterium; s_{—}
k_Bacteria; p_Actinobacteria; c_Actinobacteria; o_Actinomycetales;
f_Micrococcaceae; g_Rothia; s_mucilaginosa
k_Bacteria; p_Actinobacteria; c_Actinobacteria; o_Bifidobacteriales;
f_Bifidobacteriaceae; g_Alloscardovia; s_{—}
k_Bacteria; p_Actinobacteria; c_Actinobacteria; o_Bifidobacteriales;
f_Bifidobacteriaceae; g_Bifidobacterium
k_Bacteria; p_Actinobacteria; c_Actinobacteria; o_Bifidobacteriales;
f_Bifidobacteriaceae; g_Bifidobacterium; s_{—}
k_Bacteria; p_Actinobacteria; c_Actinobacteria; o_Bifidobacteriales;
f_Bifidobacteriaceae; g_Bifidobacterium; s_adolescentis
k_Bacteria; p_Actinobacteria; c_Coriobacteriia; o_Coriobacteriales;
f_Coriobacteriaceae
k_Bacteria; p_Actinobacteria; c_Coriobacteriia; o_Coriobacteriales;
f_Coriobacteriaceae; g_; s_{—}
k_Bacteria; p_Actinobacteria; c_Coriobacteriia; o_Coriobacteriales;
f_Coriobacteriaceae; g_Adlercreutzia; s_{—}
k_Bacteria; p_Actinobacteria; c_Coriobacteriia; o_Coriobacteriales;
f_Coriobacteriaceae; g_Atopobium; s_{—}
k_Bacteria; p_Actinobacteria; c_Coriobacteriia; o_Coriobacteriales;
f_Coriobacteriaceae; g_Collinsella; s_{—}
k_Bacteria; p_Actinobacteria; c_Coriobacteriia; o_Coriobacteriales;
f_Coriobacteriaceae; g_Collinsella; s_aerofaciens
k_Bacteria; p_Actinobacteria; c_Coriobacteriia; o_Coriobacteriales;
f_Coriobacteriaceae; g_Collinsella; s_stercoris
k_Bacteria; p_Actinobacteria; c_Coriobacteriia; o_Coriobacteriales;
f_Coriobacteriaceae; g_Eggerthella; s_lenta
k_Bacteria; p_Actinobacteria; c_Coriobacteriia; o_Coriobacteriales;
f_Coriobacteriaceae; g_Paraeggerthella; s_hongkongensis
k_Bacteria; p_Actinobacteria; c_Coriobacteriia; o_Coriobacteriales;
f_Coriobacteriaceae; g_Slackia; s_{—}
k_Bacteria; p_Bacteroidetes; c_Bacteroidia; o_Bacteroidales
k_Bacteria; p_Bacteroidetes; c_Bacteroidia; o_Bacteroidales; f_; g_; s_{—}
k_Bacteria; p_Bacteroidetes; c_Bacteroidia; o_Bacteroidales; f_[Barnesiellaceae];
g_; s_{—}
k_Bacteria; p_Bacteroidetes; c_Bacteroidia; o_Bacteroidales;
f_[Paraprevotellaceae]; g_[Prevotella]; s_{—}
k_Bacteria; p_Bacteroidetes; c_Bacteroidia; o_Bacteroidales;
f_[Paraprevotellaceae]; g_Paraprevotella; s_{—}
k_Bacteria; p_Bacteroidetes; c_Bacteroidia; o_Bacteroidales;
f_[Paraprevotellaceae]; g_YRC22; s_{—}
k_Bacteria; p_Bacteroidetes; c_Bacteroidia; o_Bacteroidales; f_Bacteroidaceae;
g_Bacteroides
k_Bacteria; p_Bacteroidetes; c_Bacteroidia; o_Bacteroidales; f_Bacteroidaceae;
g_Bacteroides; s_{—}
k_Bacteria; p_Bacteroidetes; c_Bacteroidia; o_Bacteroidales; f_Bacteroidaceae;
g_Bacteroides; s_caccae
k_Bacteria; p_Bacteroidetes; c_Bacteroidia; o_Bacteroidales; f_Bacteroidaceae;
g_Bacteroides; s_eggerthii
k_Bacteria; p_Bacteroidetes; c_Bacteroidia; o_Bacteroidales; f_Bacteroidaceae;
g_Bacteroides; s_fragilis
k_Bacteria; p_Bacteroidetes; c_Bacteroidia; o_Bacteroidales; f_Bacteroidaceae;
g_Bacteroides; s_ovatus
k_Bacteria; p_Bacteroidetes; c_Bacteroidia; o_Bacteroidales; f_Bacteroidaceae;
g_Bacteroides; s_uniformis
k_Bacteria; p_Bacteroidetes; c_Bacteroidia; o_Bacteroidales;
f_Porphyromonadaceae; g_Dysgonomonas; s_{—}
k_Bacteria; p_Bacteroidetes; c_Bacteroidia; o_Bacteroidales;
f_Porphyromonadaceae; g_Parabacteroides
k_Bacteria; p_Bacteroidetes; c_Bacteroidia; o_Bacteroidales;
f_Porphyromonadaceae; g_Parabacteroides; s_{—}
k_Bacteria; p_Bacteroidetes; c_Bacteroidia; o_Bacteroidales;
f_Porphyromonadaceae; g_Parabacteroides; s_distasonis
k_Bacteria; p_Bacteroidetes; c_Bacteroidia; o_Bacteroidales;
f_Porphyromonadaceae; g_Parabacteroides; s_gordonii
k_Bacteria; p_Bacteroidetes; c_Bacteroidia; o_Bacteroidales;
f_Porphyromonadaceae; g_Porphyromonas; s_{—}
k_Bacteria; p_Bacteroidetes; c_Bacteroidia; o_Bacteroidales; f_Prevotellaceae
k_Bacteria; p_Bacteroidetes; c_Bacteroidia; o_Bacteroidales; f_Prevotellaceae;
g_Prevotella; s_{—}
k_Bacteria; p_Bacteroidetes; c_Bacteroidia; o_Bacteroidales; f_Prevotellaceae;
g_Prevotella; s_copri
k_Bacteria; p_Bacteroidetes; c_Bacteroidia; o_Bacteroidales; f_Rikenellaceae; g_;
s_{—}
k_Bacteria; p_Bacteroidetes; c_Bacteroidia; o_Bacteroidales; f_Rikenellaceae;
g_Alistipes; s_indistinctus
k_Bacteria; p_Bacteroidetes; c_Bacteroidia; o_Bacteroidales; f_S24-7; g_; s_{—}
k_Bacteria; p_Chlamydiae; c_Chlamydiia; o_Chlamydiales; f_Chlamydiaceae;
g_Chlamydia; s_{—}
k_Bacteria; p_Cyanobacteria; c_4C0d-2; o_YS2; f_; g_; s_{—}
k_Bacteria; p_Cyanobacteria; c_Chloroplast; o_; f_; g_; s_{—}
k_Bacteria; p_Deferribacteres; c_Deferribacteres; o_Deferribacterales;
f_Deferribacteraceae; g_Mucispirillum; s_schaedleri
k_Bacteria; p_Firmicutes
k_Bacteria; p_Firmicutes; c_Bacilli; o_Bacillales; f_Bacillaceae; g_Geobacillus
k_Bacteria; p_Firmicutes; c_Bacilli; o_Bacillales; f_Bacillaceae; g_Geobacillus;
s_{—}
k_Bacteria; p_Firmicutes; c_Bacilli; o_Gemellales; f_Gemellaceae
k_Bacteria; p_Firmicutes; c_Bacilli; o_Lactobacillales
k_Bacteria; p_Firmicutes; c_Bacilli; o_Lactobacillales; f_Carnobacteriaceae;
g_Carnobacterium; s_viridans
k_Bacteria; p_Firmicutes; c_Bacilli; o_Lactobacillales; f_Carnobacteriaceae;
g_Granulicatella; s_{—}
k_Bacteria; p_Firmicutes; c_Bacilli; o_Lactobacillales; f_Enterococcaceae;
g_Enterococcus; s_{—}
k_Bacteria; p_Firmicutes; c_Bacilli; o_Lactobacillales; f_Lactobacillaceae
k_Bacteria; p_Firmicutes; c_Bacilli; o_Lactobacillales; f_Lactobacillaceae; g_; s_{—}
k_Bacteria; p_Firmicutes; c_Bacilli; o_Lactobacillales; f_Lactobacillaceae;
g_Lactobacillus; s_{—}
k_Bacteria; p_Firmicutes; c_Bacilli; o_Lactobacillales; f_Lactobacillaceae;
g_Lactobacillus; s_brevis
k_Bacteria; p_Firmicutes; c_Bacilli; o_Lactobacillales; f_Lactobacillaceae;
g_Lactobacillus; s_delbrueckii
k_Bacteria; p_Firmicutes; c_Bacilli; o_Lactobacillales; f_Lactobacillaceae;
g_Lactobacillus; s_helveticus
k_Bacteria; p_Firmicutes; c_Bacilli; o_Lactobacillales; f_Lactobacillaceae;
g_Lactobacillus; s_mucosae
k_Bacteria; p_Firmicutes; c_Bacilli; o_Lactobacillales; f_Lactobacillaceae;
g_Lactobacillus; s_reuteri
k_Bacteria; p_Firmicutes; c_Bacilli; o_Lactobacillales; f_Lactobacillaceae;
g_Lactobacillus; s_salivarius
k_Bacteria; p_Firmicutes; c_Bacilli; o_Lactobacillales; f_Lactobacillaceae;
g_Lactobacillus; s_zeae
k_Bacteria; p_Firmicutes; c_Bacilli; o_Lactobacillales; f_Lactobacillaceae;
g_Pediococcus; s_{—}
k_Bacteria; p_Firmicutes; c_Bacilli; o_Lactobacillales; f_Leuconostocaceae;
g_Leuconostoc; s_{—}
k_Bacteria; p_Firmicutes; c_Bacilli; o_Lactobacillales; f_Leuconostocaceae;
g_Leuconostoc; s_mesenteroides
k_Bacteria; p_Firmicutes; c_Bacilli; o_Lactobacillales; f_Streptococcaceae;
g_Lactococcus; s_{—}
k_Bacteria; p_Firmicutes; c_Bacilli; o_Lactobacillales; f_Streptococcaceae;
g_Streptococcus
k_Bacteria; p_Firmicutes; c_Bacilli; o_Lactobacillales; f_Streptococcaceae;
g_Streptococcus; s_{—}
k_Bacteria; p_Firmicutes; c_Bacilli; o_Lactobacillales; f_Streptococcaceae;
g_Streptococcus; s_luteciae
k_Bacteria; p_Firmicutes; c_Bacilli; o_Turicibacterales; f_Turicibacteraceae;
g_Turicibacter; s_{—}
k_Bacteria; p_Firmicutes; c_Clostridia
k_Bacteria; p_Firmicutes; c_Clostridia; o_Clostridiales
k_Bacteria; p_Firmicutes; c_Clostridia; o_Clostridiales; f_; g_; s_{—}
k_Bacteria; p_Firmicutes; c_Clostridia; o_Clostridiales; f_[Acidaminobacteraceae];
g_; s_{—}
k_Bacteria; p_Firmicutes; c_Clostridia; o_Clostridiales; f_[Mogibacteriaceae]; g_;
s_{—}
k_Bacteria; p_Firmicutes; c_Clostridia; o_Clostridiales; f_[Mogibacteriaceae];
g_Mogibacterium; s_{—}
k_Bacteria; p_Firmicutes; c_Clostridia; o_Clostridiales; f_[Tissierellaceae]; g_; s_{—}
k_Bacteria; p_Firmicutes; c_Clostridia; o_Clostridiales; f_[Tissierellaceae];
g_Anaerococcus; s_{—}
k_Bacteria; p_Firmicutes; c_Clostridia; o_Clostridiales; f_[Tissierellaceae];
g_Finegoldia; s_{—}
k_Bacteria; p_Firmicutes; c_Clostridia; o_Clostridiales; f_[Tissierellaceae];
g_Parvimonas; s_{—}
k_Bacteria; p_Firmicutes; c_Clostridia; o_Clostridiales; f_[Tissierellaceae];
g_Peptoniphilus; s_{—}
k_Bacteria; p_Firmicutes; c_Clostridia; o_Clostridiales; f_Christensenellaceae; g_;
s_{—}
k_Bacteria; p_Firmicutes; c_Clostridia; o_Clostridiales; f_Clostridiaceae
k_Bacteria; p_Firmicutes; c_Clostridia; o_Clostridiales; f_Clostridiaceae; g_; s_{—}
k_Bacteria; p_Firmicutes; c_Clostridia; o_Clostridiales; f_Clostridiaceae;
g_Clostridium
k_Bacteria; p_Firmicutes; c_Clostridia; o_Clostridiales; f_Clostridiaceae;
g_Clostridium; s_{—}
k_Bacteria; p_Firmicutes; c_Clostridia; o_Clostridiales; f_Clostridiaceae;
g_Clostridium; s_butyricum
k_Bacteria; p_Firmicutes; c_Clostridia; o_Clostridiales; f_Clostridiaceae;
g_Clostridium; s_neonatale
k_Bacteria; p_Firmicutes; c_Clostridia; o_Clostridiales; f_Clostridiaceae;
g_Clostridium; s_perfringens
k_Bacteria; p_Firmicutes; c_Clostridia; o_Clostridiales; f_Clostridiaceae;
g_SMB53; s_{—}
k_Bacteria; p_Firmicutes; c_Clostridia; o_Clostridiales; f_Dehalobacteriaceae;
g_Dehalobacterium; s_{—}
k_Bacteria; p_Firmicutes; c_Clostridia; o_Clostridiales; f_Eubacteriaceae;
g_Pseudoramibacter_Eubacterium; s_{—}
k_Bacteria; p_Firmicutes; c_Clostridia; o_Clostridiales; f_Lachnospiraceae
k_Bacteria; p_Firmicutes; c_Clostridia; o_Clostridiales; f_Lachnospiraceae; g_; s_{—}
k_Bacteria; p_Firmicutes; c_Clostridia; o_Clostridiales; f_Lachnospiraceae;
g_[Ruminococcus]; s_{—}
k_Bacteria; p_Firmicutes; c_Clostridia; o_Clostridiales; f_Lachnospiraceae;
g_[Ruminococcus]; s_gnavus
k_Bacteria; p_Firmicutes; c_Clostridia; o_Clostridiales; f_Lachnospiraceae;
g_[Ruminococcus]; s_torques
k_Bacteria; p_Firmicutes; c_Clostridia; o_Clostridiales; f_Lachnospiraceae;
g_Anaerostipes; s_{—}
k_Bacteria; p_Firmicutes; c_Clostridia; o_Clostridiales; f_Lachnospiraceae;
g_Blautia
k_Bacteria; p_Firmicutes; c_Clostridia; o_Clostridiales; f_Lachnospiraceae;
g_{—}Blautia; s_{—}
k_Bacteria; p_Firmicutes; c_Clostridia; o_Clostridiales; f_Lachnospiraceae;
g_Blautia; s_obeum
k_Bacteria; p_Firmicutes; c_Clostridia; o_Clostridiales; f_Lachnospiraceae;
g_Blautia; s_producta
k_Bacteria; p_Firmicutes; c_Clostridia; o_Clostridiales; f_Lachnospiraceae;
g_Catonella; s_{—}
k_Bacteria; p_Firmicutes; c_Clostridia; o_Clostridiales; f_Lachnospiraceae;
g_Clostridium
k_Bacteria; p_Firmicutes; c_Clostridia; o_Clostridiales; f_Lachnospiraceae;
g_Clostridium; s_hathewayi
k_Bacteria; p_Firmicutes; c_Clostridia; o_Clostridiales; f_Lachnospiraceae;
g_Clostridium; s_piliforme
k_Bacteria; p_Firmicutes; c_Clostridia; o_Clostridiales; f_Lachnospiraceae;
g_Coprococcus; s_{—}
k_Bacteria; p_Firmicutes; c_Clostridia; o_Clostridiales; f_Lachnospiraceae;
g_Coprococcus; s_catus
k_Bacteria; p_Firmicutes; c_Clostridia; o_Clostridiales; f_Lachnospiraceae;
g_Coprococcus; s_eutactus
k_Bacteria; p_Firmicutes; c_Clostridia; o_Clostridiales; f_Lachnospiraceae;
g_Dorea
k_Bacteria; p_Firmicutes; c_Clostridia; o_Clostridiales; f_Lachnospiraceae;
g_Dorea; s_{—}
k_Bacteria; p_Firmicutes; c_Clostridia; o_Clostridiales; f_Lachnospiraceae;
g_Dorea; s_formicigenerans
k_Bacteria; p_Firmicutes; c_Clostridia; o_Clostridiales; f_Lachnospiraceae;
g_Epulopiscium; s_{—}
k_Bacteria; p_Firmicutes; c_Clostridia; o_Clostridiales; f_Lachnospiraceae;
g_Lachnoanaerobaculum; s_orale
k_Bacteria; p_Firmicutes; c_Clostridia; o_Clostridiales; f_Lachnospiraceae;
g_Lachnobacterium; s_{—}
k_Bacteria; p_Firmicutes; c_Clostridia; o_Clostridiales; f_Lachnospiraceae;
g_Lachnospira; s_{—}
k_Bacteria; p_Firmicutes; c_Clostridia; o_Clostridiales; f_Lachnospiraceae;
g_Moryella; s_indoligenes
k_Bacteria; p_Firmicutes; c_Clostridia; o_Clostridiales; f_Lachnospiraceae;
g_Oribacterium; s_{—}
k_Bacteria; p_Firmicutes; c_Clostridia; o_Clostridiales; f_Lachnospiraceae;
g_Roseburia
k_Bacteria; p_Firmicutes; c_Clostridia; o_Clostridiales; f_Lachnospiraceae;
g_Roseburia; s_{—}
k_Bacteria; p_Firmicutes; c_Clostridia; o_Clostridiales; f_Lachnospiraceae;
g_Roseburia; s_faecis
k_Bacteria; p_Firmicutes; c_Clostridia; o_Clostridiales; f_Lachnospiraceae;
g_Roseburia; s_inulinivorans
k_Bacteria; p_Firmicutes; c_Clostridia; o_Clostridiales; f_Lachnospiraceae;
g_Shuttleworthia; s_{—}
k_Bacteria; p_Firmicutes; c_Clostridia; o_Clostridiales; f_Peptococcaceae; g_; s_{—}
k_Bacteria; p_Firmicutes; c_Clostridia; o_Clostridiales; f_Peptococcaceae; g_rc4-4; s_{—}
k_Bacteria; p_Firmicutes; c_Clostridia; o_Clostridiales; f_Peptostreptococcaceae;
g_; s_{—}
k_Bacteria; p_Firmicutes; c_Clostridia; o_Clostridiales; f_Peptostreptococcaceae;
g_Peptostreptococcus; s_{—}
k_Bacteria; p_Firmicutes; c_Clostridia; o_Clostridiales; f_Peptostreptococcaceae;
g_Peptostreptococcus; s_anaerobius
k_Bacteria; p_Firmicutes; c_Clostridia; o_Clostridiales; f_Ruminococcaceae
k_Bacteria; p_Firmicutes; c_Clostridia; o_Clostridiales; f_Ruminococcaceae; g_;
s_{—}
k_Bacteria; p_Firmicutes; c_Clostridia; o_Clostridiales; f_Ruminococcaceae;
g_Anaerofilum; s_{—}
k_Bacteria; p_Firmicutes; c_Clostridia; o_Clostridiales; f_Ruminococcaceae;
g_Anaerotruncus; s_{—}
k_Bacteria; p_Firmicutes; c_Clostridia; o_Clostridiales; f_Ruminococcaceae;
g_Butyricicoccus; s_pullicaecorum
k_Bacteria; p_Firmicutes; c_Clostridia; o_Clostridiales; f_Ruminococcaceae;
g_Faecalibacterium; s_{—}
k_Bacteria; p_Firmicutes; c_Clostridia; o_Clostridiales; f_Ruminococcaceae;
g_Faecalibacterium; s_prausnitzii
k_Bacteria; p_Firmicutes; c_Clostridia; o_Clostridiales; f_Ruminococcaceae;
g_Oscillospira; s_{—}
k_Bacteria; p_Firmicutes; c_Clostridia; o_Clostridiales; f_Ruminococcaceae;
g_Ruminococcus
k_Bacteria; p_Firmicutes; c_Clostridia; o_Clostridiales; f_Ruminococcaceae;
g_Ruminococcus; s_{—}
k_Bacteria; p_Firmicutes; c_Clostridia; o_Clostridiales; f_Ruminococcaceae;
g_Ruminococcus; s _albus
k_Bacteria; p_Firmicutes; c_Clostridia; o_Clostridiales; f_Ruminococcaceae;
g_Ruminococcus; s_bromii
k_Bacteria; p_Firmicutes; c_Clostridia; o_Clostridiales; f_Ruminococcaceae;
g_Ruminococcus; s_callidus
k_Bacteria; p_Firmicutes; c_Clostridia; o_Clostridiales; f_Ruminococcaceae;
g_Ruminococcus; s_flavefaciens
k_Bacteria; p_Firmicutes; c_Clostridia; o_Clostridiales; f_Veillonellaceae; g_; s_{—}
k_Bacteria; p_Firmicutes; c_Clostridia; o_Clostridiales; f_Veillonellaceae;
g_Acidaminococcus; s_{—}
k_Bacteria; p_Firmicutes; c_Clostridia; o_Clostridiales; f_Veillonellaceae;
g_Dialister; s_{—}
k_Bacteria; p_Firmicutes; c_Clostridia; o_Clostridiales; f_Veillonellaceae;
g_Megamonas; s_{—}
k_Bacteria; p_Firmicutes; c_Clostridia; o_Clostridiales; f_Veillonellaceae;
g_Megasphaera; s_{—}
k_Bacteria; p_Firmicutes; c_Clostridia; o_Clostridiales; f_Veillonellaceae;
g_Phascolarctobacterium; s_{—}
k_Bacteria; p_Firmicutes; c_Clostridia; o_Clostridiales; f_Veillonellaceae;
g_Succiniclasticum; s_{—}
k_Bacteria; p_Firmicutes; c_Clostridia; o_Clostridiales; f_Veillonellaceae;
g_Veillonella; s_dispar
k_Bacteria; p_Firmicutes; c_Clostridia; o_Clostridiales; f_Veillonellaceae;
g_Veillonella; s_parvula
k_Bacteria; p_Firmicutes; c_Clostridia; o_Thermoanaerobacterales;
f_Caldicellulosiruptoraceae; g_Caldicellulosiruptor; s_saccharolyticus
k_Bacteria; p_Firmicutes; c_Erysipelotrichi; o_Erysipelotrichales;
f_Erysipelotrichaceae
k_Bacteria; p_Firmicutes; c_Erysipelotrichi; o_Erysipelotrichales;
f_Erysipelotrichaceae; g_; s_{—}
k_Bacteria; p_Firmicutes; c_Erysipelotrichi; o_Erysipelotrichales;
f_Erysipelotrichaceae; g_[Eubacterium]; s_{—}
k_Bacteria; p_Firmicutes; c_Erysipelotrichi; o_Erysipelotrichales;
f_Erysipelotrichaceae; g_[Eubacterium]; s_biforme
k_Bacteria; p_Firmicutes; c_Erysipelotrichi; o_Erysipelotrichales;
f_Erysipelotrichaceae; g_[Eubacterium]; s_dolichum
k_Bacteria; p_Firmicutes; c_Erysipelotrichi; o_Erysipelotrichales;
f_Erysipelotrichaceae; g_Allobaculum; s_{—}
k_Bacteria; p_Firmicutes; c_Erysipelotrichi; o_Erysipelotrichales;
f_Erysipelotrichaceae; g_Bulleidia; s_moorei
k_Bacteria; p_Firmicutes; c_Erysipelotrichi; o_Erysipelotrichales;
f_Erysipelotrichaceae; g_Catenibacterium; s_{—}
k_Bacteria; p_Firmicutes; c_Erysipelotrichi; o_Erysipelotrichales;
f_Erysipelotrichaceae; g_cc_115; s_{—}
k_Bacteria; p_Firmicutes; c_Erysipelotrichi; o_Erysipelotrichales;
f_Erysipelotrichaceae; g_Clostridium; s_spiroforme
k_Bacteria; p_Firmicutes; c_Erysipelotrichi; o_Erysipelotrichales;
f_Erysipelotrichaceae; g_Coprobacillus; s_{—}
k_Bacteria; p_Firmicutes; c_Erysipelotrichi; o_Erysipelotrichales;
f_Erysipelotrichaceae; g_Holdemania; s_{—}
k_Bacteria; p_Fusobacteria; c_Fusobacteriia; o_Fusobacteriales; f_Fusobacteriaceae;
g_Fusobacterium; s_{—}
k_Bacteria; p_Lentisphaerae; c_[Lentisphaeria]; o_Victivallales; f_Victivallaceae;
g_; s_{—}
k_Bacteria; p_Proteobacteria; c_Alphaproteobacteria; o_Caulobacterales;
f_Caulobacteraceae; g_; s_{—}
k_Bacteria; p_Proteobacteria; c_Alphaproteobacteria; o_RF32; f_; g_; s_{—}
k_Bacteria; p_Proteobacteria; c_Alphaproteobacteria; o_Rhizobiales;
f_Methylobacteriaceae; g_; s_{—}
k_Bacteria; p_Proteobacteria; c_Alphaproteobacteria; o_Rhodospirillales;
f_Acetobacteraceae; g_Roseomonas; s_{—}
k_Bacteria; p_Proteobacteria; c_Alphaproteobacteria; o_Sphingomonadales;
f_Sphingomonadaceae; g_Sphingomonas
k_Bacteria; p_Proteobacteria; c_Betaproteobacteria; o_Burkholderiales
k_Bacteria; p_Proteobacteria; c_Betaproteobacteria; o_Burkholderiales;
f_Alcaligenaceae; g_Sutterella; s_{—}
k_Bacteria; p_Proteobacteria; c_Betaproteobacteria; o_Burkholderiales;
f_Oxalobacteraceae; g_Oxalobacter; s_formigenes
k_Bacteria; p_Proteobacteria; c_Betaproteobacteria; o_Neisseriales; f_Neisseriaceae;
g_Eikenella; s_{—}
k_Bacteria; p_Proteobacteria; c_Betaproteobacteria; o_Neisseriales; f_Neisseriaceae;
g_Neisseria
k_Bacteria; p_Proteobacteria; c_Deltaproteobacteria; o_Desulfovibrionales;
f_Desulfovibrionaceae; g_; s_{—}
k_Bacteria; p_Proteobacteria; c_Deltaproteobacteria; o_Desulfovibrionales;
f_Desulfovibrionaceae; g_Bilophila; s_{—}
k_Bacteria; p_Proteobacteria; c_Deltaproteobacteria; o_Desulfovibrionales;
f_Desulfovibrionaceae; g_Desulfovibrio; s_{—}
k_Bacteria; p_Proteobacteria; c_Epsilonproteobacteria; o_Campylobacterales;
f_Campylobacteraceae; g_Campylobacter; s_{—}
k_Bacteria; p_Proteobacteria; c_Epsilonproteobacteria; o_Campylobacterales;
f_Helicobacteraceae; g_Helicobacter; s_{—}
k_Bacteria; p_Proteobacteria; c_Gammaproteobacteria; o_Enterobacteriales;
f_Enterobacteriaceae
k_Bacteria; p_Proteobacteria; c_Gammaproteobacteria; o_Enterobacteriales;
f_Enterobacteriaceae; g_Citrobacter; s_{—}
k_Bacteria; p_Proteobacteria; c_Gammaproteobacteria; o_Enterobacteriales;
f_Enterobacteriaceae; g_Escherichia; s_coli
k_Bacteria; p_Proteobacteria; c_Gammaproteobacteria; o_Enterobacteriales;
f_Enterobacteriaceae; g_Morganella; s_morganii
k_Bacteria; p_Proteobacteria; c_Gammaproteobacteria; o_Enterobacteriales;
f_Enterobacteriaceae; g_Proteus; s_{—}
k_Bacteria; p_Proteobacteria; c_Gammaproteobacteria; o_Enterobacteriales;
f_Enterobacteriaceae; g_Serratia; s_{—}
k_Bacteria; p_Proteobacteria; c_Gammaproteobacteria; o_Enterobacteriales;
f_Enterobacteriaceae; g_Shigella
k_Bacteria; p_Proteobacteria; c_Gammaproteobacteria; o_Enterobacteriales;
f_Enterobacteriaceae; g_Trabulsiella
k_Bacteria; p_Proteobacteria; c_Gammaproteobacteria; o_Pasteurellales;
f_Pasteurellaceae; g_Actinobacillus
k_Bacteria; p_Proteobacteria; c_Gammaproteobacteria; o_Pasteurellales;
f_Pasteurellaceae; g_Aggregatibacter; s_{—}
k_Bacteria; p_Proteobacteria; c_Gammaproteobacteria; o_Pasteurellales;
f_Pasteurellaceae; g_Aggregatibacter; s_segnis
k_Bacteria; p_Proteobacteria; c_Gammaproteobacteria; o_Pasteurellales;
f_Pasteurellaceae; g_Haemophilus; s_parainfluenzae
k_Bacteria; p_Proteobacteria; c_Gammaproteobacteria; o_Pseudomonadales;
f_Pseudomonadaceae; g_Pseudomonas
k_Bacteria; p_Proteobacteria; c_Gammaproteobacteria; o_Pseudomonadales;
f_Pseudomonadaceae; g_Pseudomonas; s_fragi
k_Bacteria; p_Proteobacteria; c_Gammaproteobacteria; o_Pseudomonadales;
f_Pseudomonadaceae; g_Pseudomonas; s_veronii
k_Bacteria; p_Synergistetes; c_Synergistia; o_Synergistales;
f_Dethiosulfovibrionaceae; g_Pyramidobacter; s_{—}
k_Bacteria; p_Tenericutes; c_Mollicutes; o_RF39; f_; g_; s_{—}
k_Bacteria; p_Tenericutes; c_RF3; o_ML615J-28; f_; g_; s_{—}
k_Bacteria; p_Verrucomicrobia; c_Verrucomicrobiae; o_Verrucomicrobiales;
f_Verrucomicrobiaceae; g_Akkermansia; s_muciniphila
g_Anaerostipes
g_bifidobacterium
g_butyricicoccus
g_coprococcus
g_Fusicatenibacter

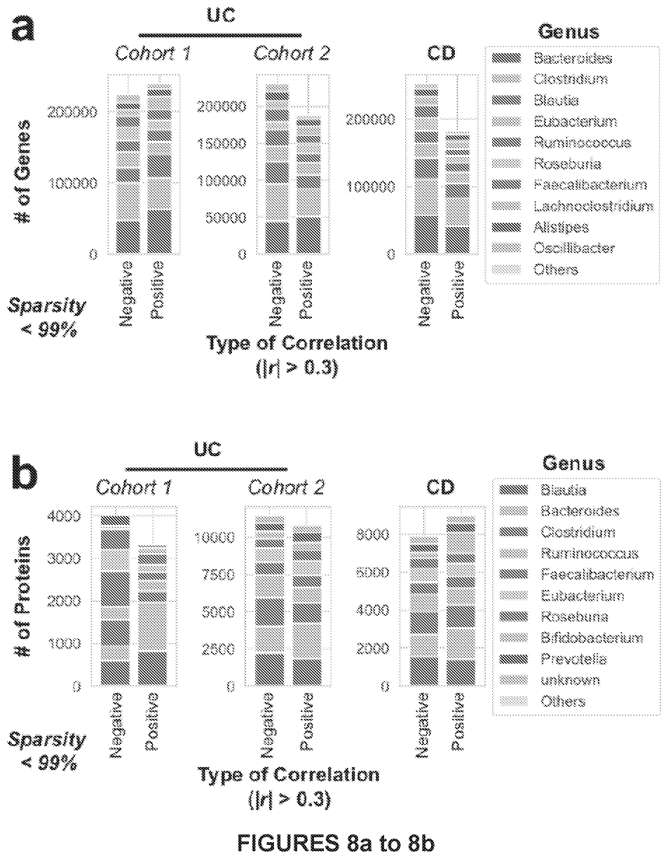

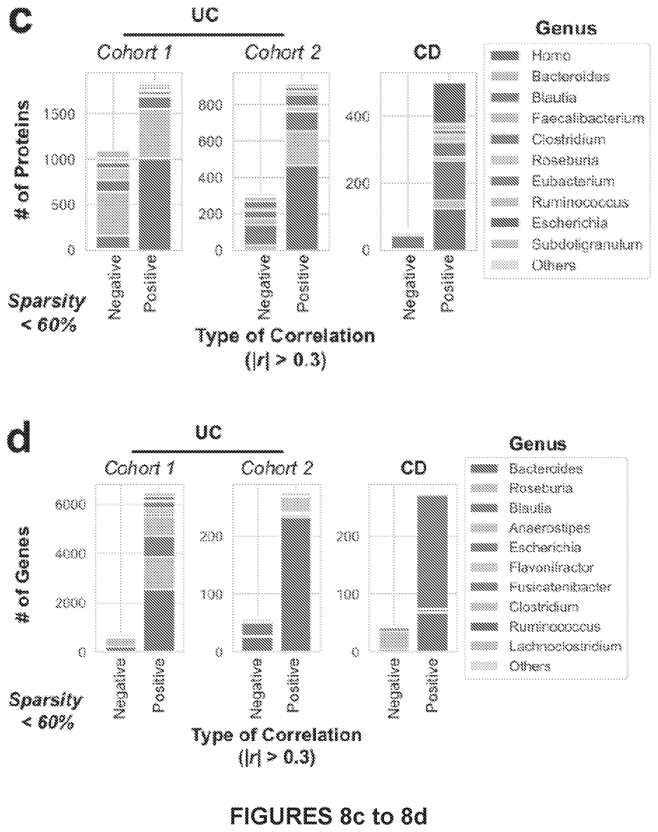

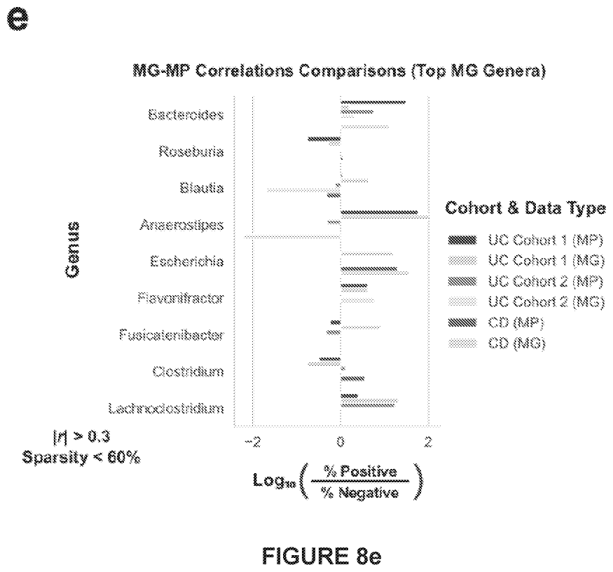

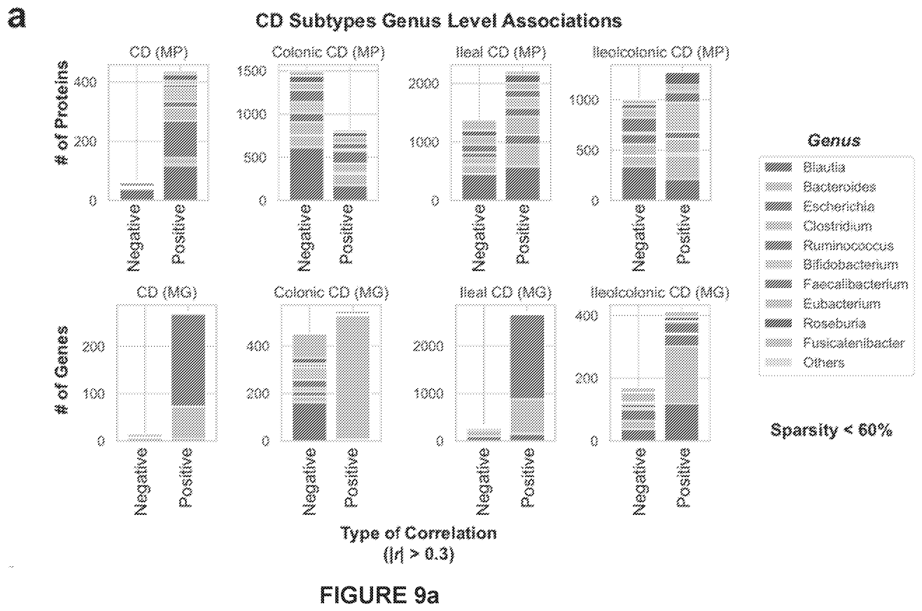

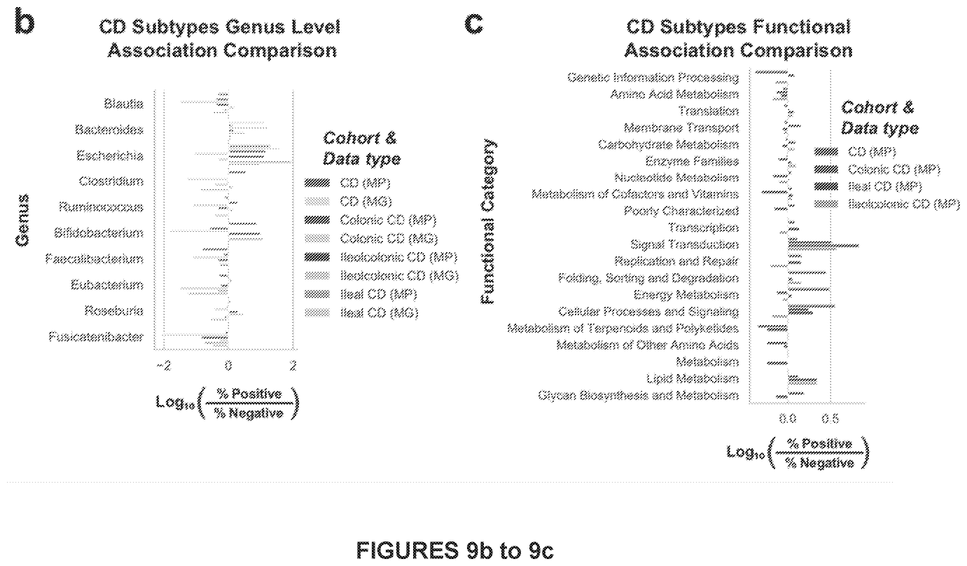

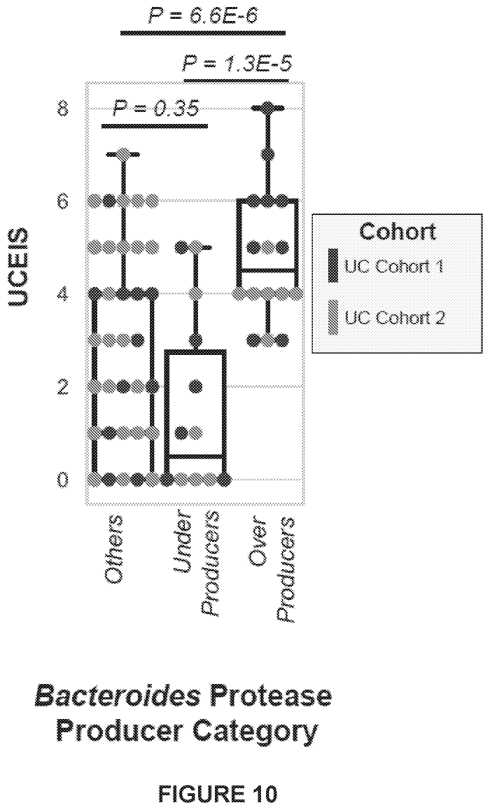

Applying a comparative metagenomic-metaproteomic approach¹⁵, linear regression identified individual genes and proteins correlated to clinical disease severity (r >0.3). Comparing genera annotations of positive and negative associations identified a clear trend of Bacteroides proteins being responsible for 40-60% of proteins positively correlated to UC disease activity (FIGS. 7a and 8). This association between disease activity and Bacteroides was confirmed across both UC cohorts, and identified as unique to UC with CD subtypes presenting unique profiles of disease correlated proteins (FIG. 9). The metagenome largely reflected the direction and magnitude of the genera level bias of the associations identified in the metaproteome, however, Bacteroides genes showed a weaker relationship to high disease severity in UC relative to the metaproteome (FIGS. 7b and 8). Functionally, proteins associated with disease activity from Bacteroides displayed an increased representation of enzyme families, and more specifically, “peptidases” (FIG. 7c). B. vulgatus and B. dorei, two closely related species prevalent among healthy subjects^{18,19}, contributed ˜40% of all Bacteroides reads in the metagenome of UC patients (FIG. 7d). From UC cohort 1, there were 45 distinct proteases derived from 34 species of Bacteroides. These proteases were grouped by class, revealing 10 serine, 9 metallo, and 4 cysteine peptidases with a range of activities including 5 di-peptidases, an endopeptidase, sialidase and signal peptidase (FIG. 7e). To test whether peptidase abundances higher than expected from the MG abundance of B vulgatus and B. dorei related to UC disease activity, Applicant applied an outlier approach to identify patient samples with over- or under-production of B. vulgatus and B. dorei proteases. This analysis showed that patients containing increased peptidases had significantly higher clinical severity and endoscopic activity in comparison to the decreased peptidase group and the typical UC patient sample (FIGS. 7f and 10). From a histological perspective, only 18.8% of patients categorized as “overproducers” were in histological remission, while 38.5% of patients categorized as “underproducers” and 45% of all other patients were in histological remission (remission defined here as Geboes Grade 3=0). As some of the correlated proteases included serine and metalloproteases, classes of proteases that largely function in the extracellular space²⁰, Applicant hypothesized that these proteins can play roles in extracellular proteolysis and exacerbation of disease activity.

Assessing Proteolysis in UC Patient-Omics and Bacteroides Supernatant

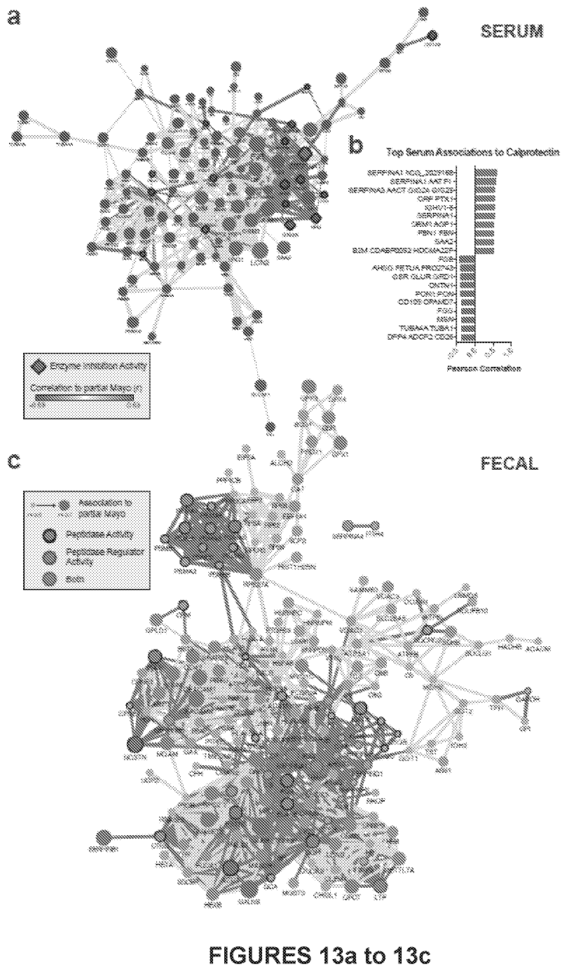

Metabolomics data corroborated the importance of proteolysis in UC patients. This was initially observed through the identification of a general increase in dipeptide abundance as one of the top 10 metabolite classes correlated with disease activity (FIGS. 3h and 11a). Dipeptides and oligopeptides were the two most common chemical classes among the metabolites positively correlated to disease activity (r >0.3; 33% and 6% respectively), and dipeptide abundance correlated with overproduction of B. vulgatus proteases (FIG. 12a). To further analyze oligopeptides, a de novo identification of the metapeptidome (short peptide fragments from multiple species) was performed²¹. Results indicated more oligopeptides within high severity UC fecal samples and patients with overproduction of Bacteroides proteases (FIGS. 11b and 12), and the identity of peptide fragments from human proteins, including structural proteins from collagens and mucins, that can be the targets of proteases (FIG. 11c). The known cleavage patterns of Neutrophil elastase and Proteinase-3²² were not strong signals among termini of identified peptides (FIG. 12c), indicating that neutrophil proteases cannot be the primary drivers of proteolysis. Network analysis of top proteins correlated to disease activity from the fecal and serum of UC patients highlighted regulation of proteolysis as a common function (FIG. 13).

To characterize the protease activity present in the Bacteroides species we identified as related to UC disease activity, bacterial cultures were grown and the supernatant was analyzed through proteomics and protease activity assays. Inhibition of serine proteases proved to be most effective for blocking active proteases in B. vulgatus supernatant (FIG. 11d). Proteomic analysis identified that serine-type activity was the most common class of enzymatic function from proteins in the supernatant of B. vulgatus, B. dorei, and B. theta (FIG. 11e). To prioritize future studies of Bacteroides proteases relevant to UC, identified proteases have been ranked by increased abundance in the supernatant of B. vulgatus compared to B. theta (FIG. 11f), ranked by the summed correlation values in UC cohorts (FIG. 11g) and a comparison of the identities of Bacteroides proteases correlated to UC patients and those found in the supernatant has been performed (FIG. 11h, Table 4).

TABLE 4
Bacteroides proteases identified in UC patients, bacterial
supernatants, and fecal material from humanized mice.
Significantly Correlated to Disease in UC Patients (r > 0.3)

| UC Cohort 1 | UC Cohort 2 | UC Cohort 1 & 2 | UC Cohort 1 or 2 |
|---|---|---|---|
| Aminopeptidase C | Aminoacyl-histidine | Aminopeptidase C | Aminoacyl-histidine |
| | dipeptidase (Peptidase D) | | dipeptidase (Peptidase D) |
| | (Fragment) | | (Fragment) |
| Aminopeptidase P | Aminopeptidase C | Dipeptidase | Aminopeptidase C |
| domain protein | | (EC 3.4.—.—) | |
| (EC 3.4.—.—) | | | |
| Carboxypeptidase | Aminopeptidase C | Dipeptidyl | Aminopeptidase C |
| regulatory-like domain | (Bleomycin hydrolase) | aminopeptidase IV | (Bleomycin hydrolase) |
| protein (TonB-dependent | | | |
| Receptor Plug Domain | | | |
| protein) | | | |
| Dipeptidase | ATP-dependent zinc | Dipeptidyl | Aminopeptidase P |
| (EC 3.4.—.—) | metalloprotease FtsH | peptidase IV | domain protein |
| | (EC3.4.24.—) | | (EC 3.4.—.—) |
| Dipeptidyl | Dipeptidase | Peptidase S9A/B/C | ATP-dependent zinc |
| aminopeptidase IV | (EC 3.4.—.—) | family catalytic | metalloprotease FtsH |
| | | domain protein | (EC 3.4.24.—) |
| Dipeptidyl | Dipeptidyl | Putative Tricorn- | Carboxypeptidase |
| peptidase IV | aminopeptidase IV | like protease | regulatory-like domain |
| | | | protein (TonB-dependent |
| | | | Receptor Plug Domain |
| | | | protein) |
| Endothelin-converting | Dipeptidyl | Tricorn protease | Dipeptidase |
| enzyme 1 (EC 3.4.24.71) | peptidase IV | | (EC 3.4.—.—) |
| Peptidase C1-like family | Dipeptidyl-peptidase 7 | Tricorn protease | Dipeptidyl |
| (EC 3.4.22.—) | (DPP7) | homolog | aminopeptidase IV |
| | (EC3.4.14.—) | (EC 3.4.21.—) | |
| Peptidase C1A papain | Leucine | | Dipeptidyl |
| | aminopeptidase | | peptidase IV |
| Peptidase S24-like protein | Peptidase M16 inactive | | Dipeptidyl-peptidase 7 |
| | domain protein | | (DPP7) |
| | (EC 3.4.24.—) | | (EC 3.4.14.—) |
| Peptidase S9A/B/C | Peptidase S41 family | | Endothelin-converting |
| family catalytic | | | enzyme 1 (EC 3.4.24.71) |
| domain protein | | | |
| Peptidase, M28 family | Peptidase S9A/B/C | | Leucine aminopeptidase |
| (EC 3.4.—.—) | family catalytic | | |
| | domain protein | | |
| Protease Do | Peptidase_S9 domain- | | Peptidase C1-like family |
| | containing protein | | (EC 3.4.22.—) |
| Putative Tricorn-like | Peptidase, S41 family | | Peptidase C1A papain |
| protease | | | |
| Serine protease | Peptidase, S9A/B/C | | Peptidase M16 inactive |
| | family, catalytic | | domain protein (EC |
| | domain protein | | 3.4.24.—) |
| | (EC 3.4.—.—) | | |
| Signal peptide peptidase | Putative Tricorn-like | | Peptidase S24-like protein |
| SppA, 67K type | protease | | |
| (EC 3.4.—.—) | | | |
| Tricorn protease | Renal dipeptidase | | Peptidase S41 family |
| | family protein | | |
| Tricorn protease homolog | Tricorn protease | | Peptidase S9A/B/C family |
| (EC 3.4.21.—) | | | catalytic domain protein |
| Xaa-His dipeptidase | Tricorn protease homolog | | Peptidase_S9 domain- |
| (EC3.4.13.3) | (EC 3.4.21.—) | | containing protein |
| | | | Peptidase, M28 family |
| | | | (EC 3.4.—.—) |
| | | | Peptidase, S41 family |
| | | | Peptidase, S9A/B/C |
| | | | family, catalytic |
| | | | domain protein |
| | | | (EC 3.4.—.—) |
| | | | Protease Do |
| | | | Putative Tricorn-like |
| | | | protease |
| | | | Renal dipeptidase family |
| | | | protein |
| | | | Serine protease |
| | | | Signal peptide peptidase |
| | | | SppA, 67K type |
| | | | (EC 3.4.—.—) |
| | | | Tricorn protease |
| | | | Tricorn protease homolog |
| | | | (EC 3.4.21.—) |
| | | | Xaa-His dipeptidase |
| | | | (EC 3.4.13.3) |

Fecal Transplants
Significant comparing overabundant to underabundant protease mice (π > 1)

Aminoacyl-histidine dipeptidase (Cytosol non-specific dipeptidase) (EC 3.4.13.18)
Aminopeptidase
Aminopeptidase P domain protein (EC 3.4.—.—)
Aminopeptidase P family protein (Putative peptidase) (EC 3.4.13.9) (Xaa-Pro dipeptidase)
ATP-dependent Clp protease ATP-binding subunit ClpX
ATP-dependent Clp protease proteolytic subunit (EC 3.4.21.92) (Endopeptidase Clp)
ATP-dependent zinc metalloprotease FtsH (EC 3.4.24.—)
Carboxyl-terminal protease
Carboxypeptidase regulatory-like domain protein
Carboxypeptidase regulatory-like domain protein (TonB-dependent Receptor Plug Domain
protein)
Collagenase (EC 3.4.—.—)
Dipeptidase (EC 3.4.—.—)
Dipeptidyl-peptidase (EC 3.4.14.—)
Dipeptidyl-peptidase 7 (DPP7) (EC 3.4.14.—)
Dipeptidyl-peptidase IV (EC 3.4.14.5)
LysM peptidoglycan-binding domain-containing protein (Putative D-gamma-glutamyl-meso-
diaminopimelic acid endopeptidase, CwlS-like) (EC 3.4.19.11)
Membrane protease subunit, stomatin/prohibitin
Peptidase dimerization domain protein (EC 3.4.—.—)
Peptidase M16 inactive domain protein (EC 3.4.24.—)
Peptidase S24-like protein
Peptidase T (EC 3.4.11.4) (Aminotripeptidase) (Tripeptidase) (Tripeptide aminopeptidase)
Peptidase, M28 family (EC 3.4.—.—)
Peptidase, M48 family (EC 3.4.24.—)
Peptidase, S41 family (EC 3.4.21.—)
Peptidase, S8/S53 family (EC 3.4.21.—)
Peptidase, S9A/B/C family, catalytic domain protein (EC 3.4.—.—)
Prolyl tripeptidyl peptidase (EC 3.4.14.12)
Protease 3 (EC 3.4.24.55)
Secreted tripeptidyl aminopeptidase (Fragment)
Sensor protein RprX (EC 2.7.13.3)
Signal peptidase I (EC 3.4.21.89)
Tricorn protease homolog (EC 3.4.21.—)
TSPc domain-containing protein
Xaa-His dipeptidase (EC 3.4.13.3)
Xaa-Pro aminopeptidase (EC 3.4.11.9)
Zinc metalloprotease (EC 3.4.24.—)

Bacterial Supernatants

| | | | Identified in | |
|---|---|---|---|---|
| | | | B. vulgatus | |
| | | | or B. dorei, | Identified in |
| Identified in | Identified in | Identified in | but not in | B. vulgatus or |
| B. vulgatus | B. dorei | B. theta | B. theta | B. dorei |
| Aminoacyl- | Aminoacyl- | Aminoacyl- | ATP- | Aminoacyl- |
| histidine | histidine | histidine | dependent | histidine |
| dipeptidase | dipeptidase | dipeptidase | Clp protease | dipeptidase |
| | | | ATP-binding | |
| | | | subunit ClpX | |
| Aminopeptidase | C-terminal | Aminopeptidase | ATP- | Aminopeptidase |
| | processing | | dependent zinc | |
| | peptidase | | metalloprotease | |
| | | | FtsH | |
| | | | (EC 3.4.24.—) | |
| ATP-dependent | Dipeptidase | ATP-dependent | C-terminal | ATP-dependent |
| Clp protease | (EC 3.4.—.—) | Clp protease | processing | Clp protease |
| ATP-binding | | | peptidase | ATP-binding |
| subunit ClpX | | | | subunit ClpX |
| ATP-dependent | Dipeptidyl- | ATP-dependent | D-alanyl-D- | ATP-dependent |
| Clp protease | peptidase | Clp protease | alanine | Clp protease |
| proteolytic | (EC 3.4.14.—) | proteolytic | dipeptidase | proteolytic |
| subunit | | subunit | (D-Ala-D-Ala | subunit |
| (EC 3.4.21.92) | | (EC 3.4.21.92) | dipeptidase) | (EC 3.4.21.92) |
| (Endopeptidase | | (Endopeptidase | (EC 3.4.13.22) | (Endopeptidase |
| Clp) | | Clp) | | Clp) |
| ATP- | Lon protease | ATP-dependent | Dipeptidase | ATP- |
| dependent zinc | (EC 3.4.21.53) | protease | (EC 3.4.—.—) | dependent zinc |
| metalloprotease | (ATP-dependent | | | metalloprotease |
| FtsH | protease La) | | | FtsH |
| (EC 3.4.24.—) | | | | (EC 3.4.24.—) |
| Carboxy- | Methionine | Carboxy- | Dipeptidyl- | C-terminal |
| terminal | aminopeptidase | terminal | peptidase 7 | processing |
| processing | (MAP) (MetAP) | processing | (DPP7) | peptidase |
| protease | (EC 3.4.11.18) | protease | (EC 3.4.14.—) | |
| | (Peptidase M) | | | |
| D-alanyl-D- | Peptidase T | Carboxyl- | Glutamine | Carboxy- |
| alanine | (EC 3.4.11.4) | terminal | amidotransferase, | terminal |
| dipeptidase | (Aminotripeptidase) | protease | class II/ | processing |
| (D-Ala-D-Ala | (Tripeptidase) | | dipeptidase | protease |
| dipeptidase) | (Tripeptide | | | |
| (EC 3.4.13.22) | aminopeptidase) | | | |
| Dipeptidase | Peptidase_M15_3 | Dipeptidyl | Lon protease | D-alanyl-D- |
| (EC 3.4.—.—) | domain-containing | peptidase IV | (EC 3.4.21.53) | alanine |
| | protein | | (ATP- | dipeptidase |
| | | | dependent | (D-Ala-D-Ala |
| | | | protease La) | dipeptidase) |
| | | | | (EC 3.4.13.22) |
| Dipeptidyl | Peptidase_M3 | Dipeptidyl- | Methionine | Dipeptidase |
| peptidase IV | domain-containing | peptidase | aminopeptidase | (EC 3.4.—.—) |
| | protein | (EC 3.4.14.—) | (MAP) (MetAP) | |
| | | | (EC 3.4.11.18) | |
| | | | (Peptidase M) | |
| Dipeptidyl- | Peptidase_S24 | Dipeptidyl- | Peptidase_M15_3 | Dipeptidyl |
| peptidase | domain-containing | peptidase VI | domain-containing | peptidase IV |
| (EC 3.4.14.—) | protein | | protein | |
| Dipeptidyl- | Peptidase_S8 | Endopeptidase | Peptidase_M23 | Dipeptidyl- |
| peptidase 7 | domain-containing | La | domain-containing | peptidase |
| (DPP7) | protein | (EC 3.4.21.53) | protein | (EC 3.4.14.—) |
| (EC 3.4.14.—) | | | | |
| Glutamine | Peptidase_S9 | Leucine | Peptidase_M3 | Dipeptidyl- |
| amidotransferase, | domain-containing | aminopeptidase | domain-containing | peptidase 7 |
| class II/ | protein | | protein | (DPP7) |
| dipeptidase | | | | (EC 3.4.14.—) |
| Lon protease | Protease Do | Metallopeptidase | Peptidase_S24 | Glutamine |
| (EC 3.4.21.53) | | family M24, | domain-containing | amidotransferase, |
| (ATP-dependent | | putative Xaa-Pro | protein | class II/ |
| protease La) | | dipeptidase | | dipeptidase |
| Methionine | Tricorn protease | Peptidase | Peptidase_S8 | Lon protease |
| aminopeptidase | homolog | | domain-containing | (EC 3.4.21.53) |
| (MAP) (MetAP) | (EC 3.4.21.—) | | protein | (ATP-dependent |
| (EC 3.4.11.18) | | | | protease La) |
| (Peptidase M) | | | | |
| Peptidase | | Peptidase M60 | Peptidase_S9 | Methionine |
| | | domain-containing | domain-containing | aminopeptidase |
| | | protein | protein | (MAP) (MetAP) |
| | | | | (EC 3.4.11.18) |
| | | | | (Peptidase M) |
| Peptidase M60 | | Peptidase S26A, | Protease Do | Peptidase |
| domain-containing | | signal peptidase | | |
| protein | | | | |
| Peptidase T | | Peptidase T | Putative | Peptidase M60 |
| (EC 3.4.11.4) | | (EC 3.4.11.4) | acylaminoacyl- | domain-containing |
| (Aminotripeptidase) | | (Aminotripeptidase) | peptidase | protein |
| (Tripeptidase) | | (Tripeptidase) | | |
| (Tripeptide | | (Tripeptide | | |
| aminopeptidase) | | aminopeptidase) | | |
| Peptidase_M23 | | Peptidyl- | Putative | Peptidase T |
| domain-containing | | dipeptidase | alanyl | (EC 3.4.11.4) |
| protein | | | dipeptidyl | (Aminotripeptidase) |
| | | | peptidase | (Tripeptidase) |
| | | | | (Tripeptide |
| | | | | aminopeptidase) |
| Peptidyl- | | Prolyl | Putative | Peptidase_M15_3 |
| dipeptidase | | oligopeptidase | exported | domain-containing |
| | | family protein | serine | protein |
| | | | protease, | |
| | | | subtilase | |
| | | | family | |
| Prolyl | | Protease | Putative | Peptidase_M23 |
| oligopeptidase | | | glycoprotease | domain-containing |
| family protein | | | | protein |
| Protease IV | | Protease IV | Putative | Peptidase_M3 |
| | | | peptidase | domain-containing |
| | | | | protein |
| Putative | | Putative alkaline | Putative | Peptidase_S24 |
| acylaminoacyl- | | protease aprF | peptidase/ | domain-containing |
| peptidase | | | deacetylase | protein |
| Putative alanyl | | Putative | Putative | Peptidase_S8 |
| dipeptidyl | | aminopeptidase | peptidyl- | domain-containing |
| peptidase | | | dipeptidase | protein |
| Putative | | Putative | Putative | Peptidase_S9 |
| aminopeptidase | | aminopeptidase | protease I | domain-containing |
| | | C | | protein |
| Putative | | Putative | Putative ThiJ | Peptidyl- |
| dipeptidyl- | | dipeptidyl- | family | dipeptidase |
| peptidase III | | peptidase III | intracellular | |
| | | | protease | |
| Putative | | Putative | Putative Xaa- | Prolyl |
| exported serine | | membrane | Pro | oligopeptidase |
| protease, | | peptidase | dipeptidase | family protein |
| subtilase family | | | | |
| Putative | | Putative | Tricorn | Protease Do |
| glycoprotease | | periplasmic | protease | |
| | | protease | homolog | |
| | | | (EC 3.4.21.—) | |
| Putative | | Putative | Zinc | Protease IV |
| peptidase | | protease | metalloprotease | |
| | | | (EC 3.4.24.—) | |
| Putative | | Putative ThiJ | | Putative |
| peptidase/ | | family | | acylaminoacyl- |
| deacetylase | | intracellular | | peptidase |
| | | protease/ | | |
| | | amidase | | |
| Putative | | Putative zinc | | Putative alanyl |
| peptidyl- | | protease | | dipeptidyl |
| dipeptidase | | | | peptidase |
| Putative | | Serine protease | | Putative |
| protease I | | | | aminopeptidase |
| Putative ThiJ | | Signal peptidase | | Putative |
| family | | I (EC 3.4.21.89) | | dipeptidyl- |
| intracellular | | | | peptidase III |
| protease | | | | |
| Putative Xaa- | | Subtilisin-like | | Putative |
| Pro dipeptidase | | serine protease | | exported serine |
| | | | | protease, |
| | | | | subtilase family |
| Putative zinc | | Xaa-Pro | | Putative |
| protease | | aminopeptidase | | glycoprotease |
| Serine protease | | | | Putative |
| | | | | peptidase |
| Signal peptidase | | | | Putative |
| I (EC 3.4.21.89) | | | | peptidase/ |
| | | | | deacetylase |
| Tricorn protease | | | | Putative |
| homolog | | | | peptidyl- |
| (EC 3.4.21.—) | | | | dipeptidase |
| Xaa-Pro | | | | Putative |
| aminopeptidase | | | | protease I |
| Zinc | | | | Putative ThiJ |
| metalloprotease | | | | family |
| (EC 3.4.24.—) | | | | intracellular |
| | | | | protease |
| | | | | Putative Xaa- |
| | | | | Pro dipeptidase |
| | | | | Putative zinc |
| | | | | protease |
| | | | | Serine protease |
| | | | | Signal peptidase |
| | | | | I (EC 3.4.21.89) |
| | | | | Tricorn protease |
| | | | | homolog |
| | | | | (EC 3.4.21.—) |
| | | | | Xaa-Pro |
| | | | | aminopeptidase |
| | | | | Zinc |
| | | | | metalloprotease |
| | | | | (EC 3.4.24.—) |

Protein names are listed of peptidases or proteases identified throughout various proteomic experiments from this study. Lists are provided for any proteases from B. vulgatus or B. dorei that were positively associated (r >0.3) with UC patient disease activity from either cohort. Additionally proteases or peptidases identified in the supernatant from different species of Bacteroides are listed. Finally, from a metaproteomic analysis of the fecal material in humanized mice, B. vulgatus or B. dorei proteases increased (π>1) in mice transplanted with overabundant proteases sample H19 in comparison to mice transplanted without overabundant proteases sample L3.

TABLE 5
Number of genes and proteins correlated
to partial Mayo by functional category

| | Genes Correlated to | | Proteins Correlated to | |
|---|---|---|---|---|
| | partial Mayo | | partial Mayo | |
| Genus | Positive | Negative | Positive | Negative |
| Bacteroides | 5486 | 1283 | 528 | 5 |
| Anaerostipes | 684 | 60 | 37 | 0 |
| Unknown | 1908 | 1595 | 117 | 41 |
| Blautia | 2421 | 920 | 134 | 66 |
| Ruminococcus | 714 | 326 | 45 | 25 |
| Eubacterium | 246 | 976 | 30 | 39 |
| Clostridium | 2351 | 5607 | 48 | 74 |
| Roseburia | 480 | 3694 | 28 | 80 |
| Subdoligranulum | 16 | 360 | 21 | 59 |
| Faecalibacterium | 35 | 960 | 27 | 377 |

Linear relationships of gene or protein quantities to partial Mayo scores were assessed and the most associated genes and proteins (|r|>0.3) were compiled by genus annotation.

TABLE 6
Number of genes and proteins correlated to partial Mayo by functional category

| | Genes Correlated to | | Proteins Correlated to | |
|---|---|---|---|---|
| | partial Mayo | | partial Mayo | |
| KEGG Functional Categories | Positive | Negative | Positive | Negative |
| Amino Add Metabolism | 1077 | 831 | 92 | 86 |
| Biosynthesis of Other Secondary | 8 | 1 | 0 | 1 |
| Metabolites | | | | |
| Carbohydrate Metabolism | 1697 | 1218 | 267 | 173 |
| Cell Motility | 31 | 48 | 1 | 0 |
| Cellular Processes and Signaling | 816 | 614 | 15 | 7 |
| Energy Metabolism | 535 | 403 | 60 | 61 |
| Enzyme Families | 408 | 327 | 24 | 5 |
| Folding, Sorting and Degradation | 344 | 300 | 36 | 17 |
| Genetic Information Processing | 543 | 353 | 16 | 9 |
| Glycan Biosynthesis and Metabolism | 187 | 126 | 1 | 0 |
| Lipid Metabolism | 222 | 189 | 14 | 5 |
| Membrane Transport | 1375 | 1096 | 33 | 61 |
| Metabolism | 452 | 282 | 24 | 19 |
| Metabolism of Cofactors and Vitamins | 488 | 319 | 13 | 5 |
| Metabolism of Other Amino Acids | 119 | 71 | 6 | 3 |
| Metabolism of Terpenoids and Polyketides | 126 | 114 | 1 | 6 |
| Nucleotide Metabolism | 684 | 587 | 76 | 62 |
| Poorly Characterized | 776 | 598 | 12 | 6 |
| Replication and Repair | 882 | 723 | 28 | 7 |
| Signal Transduction | 132 | 126 | 3 | 1 |
| Transcription | 324 | 291 | 5 | 3 |
| Translation | 573 | 572 | 125 | 128 |

Table 7 provides a list of proteins listed in FIG. 13c which provides that peptidase related proteins are highly connected within fecal exosome proteins correlated to disease activity.

Peptidase Related Proteins Connected within Fecal
Exosome Proteins and Correlated to Disease Activity

HEBP2, ACAA2, TSG101, RPS5, SRP14, TMC6, AMY1A, TMED4, CHMP6, SRP9,
CCT8, TPMT, PA2G4, SHMT1, IQGAP2, ASNA1, FH, AGT, ARSB, KRT74, TFG,
BLVRA, SERPINH1, CTSA, HNRNPD, CREG1, GPX2, BROX, LAMTOR3, GOLM1,
AMY2A, PSMC6, KRT2, RAB13, FKBP5, KPNB1, CHMP2B, DNM2, TGFBI, ITIH3,
CNN2, RNASE2, RPL3, RHOA, RPL26, ATIC, REG1B, LMAN1, VPS4B, RPL5,
XPNPEP2, BSG, STXBP3, SHMT2, KRT6A, RPL15, SPTAN1, PTBP1, KRT10, EIF2S3,
ACTA1, ARHGAP1, CHMP2A, RTN4, RPS13, RAB8B, CCT6A, AKR1A1, CFL1,
CD300A, GALC, TLN1, ENPP3, TXN, COX7A2, ABI1, AKR1B10, RRAS, PAFAH1B1,
DNAJC3, ARL8A, TFRC, VCL, MYH9, SND1, RPL30, ATP6V1B2, STK10, NRAS,
S100A10, CFHR1, DYNLL1, KRT19, ACLY, YWHAQ, YWHAZ, GNG5, YWHAB,
VASP, CAD, ACTR3, ARHGDIA, CAB39L, ARSA, CDC42, ADH5, ARF4, RHOG,
COMT, IQGAP1, GSN, GM2A, SLC2A5, PRDX5, RPL27, PSME2, AMY2B, PPP2R1A,
CAND1, CDH1, PKM, CAPZA2, ACTB, UBA1, ARPC3, TUBB8, ITIH2, VCP, ACTN4,
PFKL, IDH1, SGSH, CAPZB, NAGA, PGK1, GNAI3, RAB22A, HYOU1, PGAM1,
DDX3X, MYL12A, PGD, ENO1, PRDX2, LCP1, CHMP1B, SSR4, HNRNPL, HAGH,
RAC2, RPL11, MAPK1, HSP90AB1, RAB14, BLVRB, RPL7A, NSF, NT5E, GSTP1,
VNN1, RAB9A, RAB10, VPS35, RAB7A, FLOT2, TUBB4B, RAB8A, ACAT1,
CPPED1, RPL10A, LGALS1, HNRNPA2B1, CAP1, PEF1, TUBB, HRG, RAB11B,
RAP2B, SPTBN1, PDCD6IP, RPS19, GNA13, GBE1, GNAQ, SCARB2, GLRX, CD44,
RAB33B, FABP1, DYNC1H1, ALDH6A1, CAPN5, PSMB6, LTA4H, ARPC1B, ATP5H,
COX5A, GRB2, HLA-C, MME, MT-CO2, RAB27A, TCP1, APOB, RPL14, RPL7,
PSMC5, PROS1, C4B, CCT4, PCBP1, TXNDC5, ATP8A1, ERP44, CAPNS1, RAB2A,
GSTK1, RPS25, GOLGA7, ETFA, FLOT1, GNB2, RPS4X, RPL4, PSMB2, RAP1B,
RPS11, PDHB, GDI2, RETN, CAPZA1, AK2, MT-ATP6, CAMP, PACSIN2, LRG1,
ADSS, SRSF7, STK24, SAR1A, HSPA6, PNP, NCKAP1L, C8A, FTH1, ALDOC, DDB1,
SYTL1, HPRT1, NHLRC3, NDUFA4, ACTN1, VAT1, C6, ARPC2, HSPA9, GYG1,
MMP9, ATP5F1, PABPC1, ARF6, MNDA, DNAJC13, EZR, CD177, RPS18, LYN,
PEBP1, ATP5O, CSK, ADIPOQ, CD63, HLA-DRB1, MUC4, ARPC5, MVP, ALDH3B1,
ATP6AP1, FASN, KNG1, ALB, ANPEP, EHD1, TUBB2A, APCS, PSMB4, RPS14,
B2M, PLG, MYL6, EIF4A1, CAPN2, HSPD1, DAD1, CLTC, CSTB, RBP4, TUFM,
DERA, TF, ENO3, ALPL, DNAJC5, IGF2R, C1QA, RPS3, RHOC, RAN, ATP6V1A,
MUC13, LDHB, FGR, FGA, TALDO1, FERMT3, TOMM40, TMED9, LGALS3BP, CR1,
C1S, RAB6A, MAPK14, ATP6V0D1, ATP6V0C, TMED10, EPCAM, PPP2CA, GAPDH,
PYGB, CFH, GOT1, C1QB, HSPA5, A2M, RHOF, IMPDH2, UGP2, CPNE1, USMG5,
ACADM, PRDX3, RPLP0, DOCK2, HLA-A, RNASE3, GNB1, GPI, SERPINC1, MDH2,
AKR1C3, PDIA3, RPSA, PPP1CB, F2, SERPINF2, P4HB, ATP1B1, RAP1A, CS, IDH2,
C1QC, EEF2, COX4I1, ALDH2, HADHB, EIF5A, AMBP, C4A, PIGR, VAMP8, TST,
C1R, C8G, HP, GOT2, CALR, SUCLG1, LMAN2, KRT18, VTN, CTSB, HNRNPM,
HSPA8, GCA, AHSG, ITIH4, A1BG, SERPINA5, NAPA, RPS8, F11R, CTSH, PTGES3,
PSMB8, PTPRC, TIMP2, TPI1, FCGR2A, CRISP3, ATP5L, GBA, OLFM4, FGB, RPS9,
RPS2, PSMA5, PSMB1, PHB, HLA-B, QSOX1, SAMM50, HEXA, PHB2, BPI,
NDUFB10, HIST1H4A, CHI3L1, ITGB2, CPNE3, PSAP, PPIA, PSMB9, CAPN1,
ATP1A1, VDAC1, PSMB3, ASS1, SERPING1, SNRPD2, GAA, BST1, FGG, SDCBP,
HLA-DRA, C3, GPX4, PSMA2, C5, UQCRC2, SLC25A5, MGST3, CPM, ADAM10,
PADI2, C9, SOD1, HSP90B1, ANXA1, SCP2, HIST1H2BN, HNRNPC, LSM6, C8B,
FUCA1, ORM2, TTR, C7, SERPINA4, LYZ, VDAC3, HBB, CAT, CA2, EEF1A1,
SDHB, SERPINB6, PRDX1, ESD, CP, FTL, CTSC, CTSG, PRDX4, PSMA6, PTPRJ,
SNAP23, HEXB, LCN2, MAN2B1, GPLD1, PSMB5, RPS27A, GSR, LAMP1, ORM1,
ELANE, MYO1C, PSMA7, METTL7A, TXNRD1, MPO, IGLL5, TIMP1, AGA, GPX1,
PSMA3, PRTN3, MGAM, SERPINA1, GGH, QPCT, TOLLIP, NPC2, CD47, GPX3,
LTF, PRCP, STOM, GNS, RNASET2, SERPINB1, CEACAM1, CEACAM8, AZU1,
NCSTN, GALNS, S100A9, GUSB, ATP5A1, ATP5B, ATP5C1, GNB2L1, GRN,
CHMP4B, RPS16, SLC2A3, SI, NAGLU, TUBA4A, MTHFD1, ENPEP, CPB2, GNA11,
KRT1, PPIB, SNRPD3, SERPIND1, PSMA4, RALA, RAB35, CSE1L, PGLYRP1,
APOH, EPX, ARHGDIB, PFN1, MSN, REG1A, CEACAM5, ATP1B3, ARF3, WDR1,
ACP2, PYGL, NAMPT, MSRA, HSPE1, EPS8, DCXR, ATP6V1E1, LGALS3, CTBS,
CORO1A, CAB39, FLNA, ECH1, PGLS, GLO1, SNX3, SORL1, LRRK2, TWF2,
PDCD6, CHP1, NDUFB3, DPP4, PON1, ARL8B, ST13, SLC25A3, CRP, GALNT3,
PARP4, KLKB1, HPX, LAP3, EHD4, MOGS, REEP5, IGJ, CSTA, LBP, H2AFY,
ICAM3, S100A11, ANXA6, DECR1, MEP1A, S100A8.

Linear relationships of gene or protein quantities to partial Mayo scores were assessed and the most associated genes and proteins (|r|>0.3) were compiled by function according to their KEGG Orthology annotations.

Protease Inhibition Prevents B. vulgatus Induced Colonic Epithelial Damage In Vitro and In Vivo

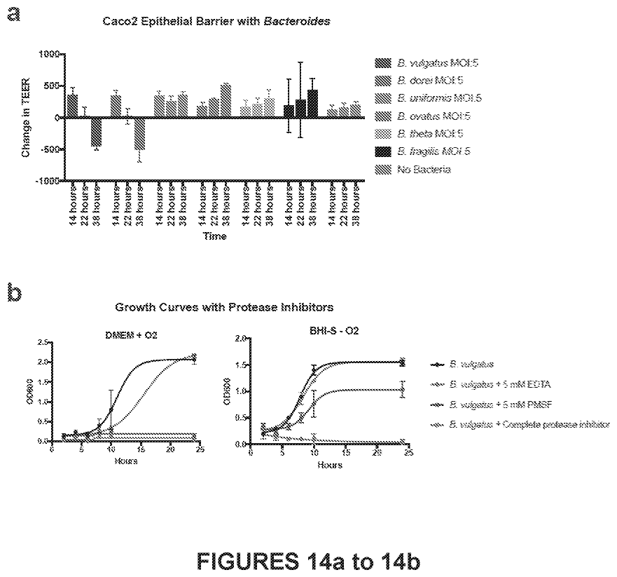

Given the enrichment of Bacteroides proteases from the meta-omics analyses as provided herein, Applicant assessed the six most abundant Bacteroides species for effects on intestinal barrier using in vitro Caco-2 epithelial monolayers. The results showed a significant decrease in transepithelial electrical resistance (TEER) after 38 hours of incubation with the two most abundant Bacteroides species Applicant identified in UC, Bacteroides vulgatus and Bacteroides dorei, while other species increased TEER (FIG. 14a). Applicant next assessed the contribution of protease activity in disruption of epithelial permeability through the addition of a protease inhibitor cocktail specific to serine and cysteine proteases. Applicant found that protease inhibition significantly increased TEER at both 22 and 38 hours post-inoculation with B. vulgatus (Adjusted p-value<0.0001, FIGS. 15a-15b). The phenotype was not due to effects on bacterial growth or viability, as colony-forming units (CFUs) were not significantly different between the B. vulgatus wells treated with or without protease inhibitor cocktail (Adjusted p-value=0.98, FIGS. 15c and 14b).

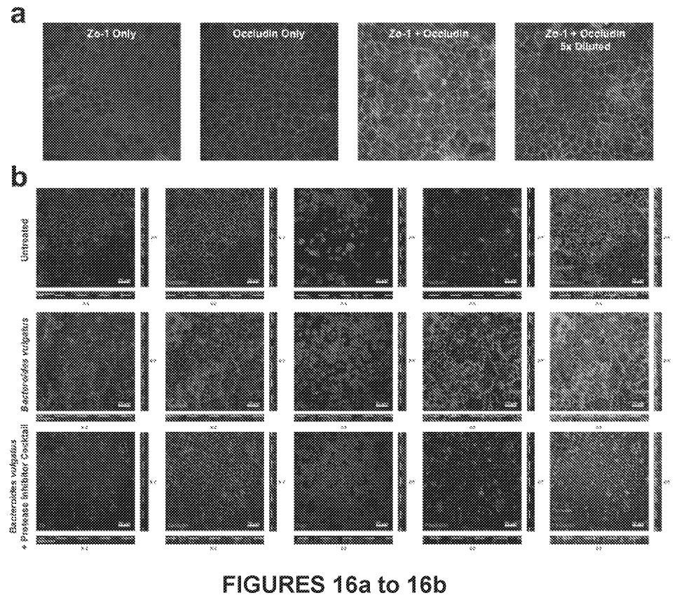

Confocal microscopy of the intestinal monolayers revealed dramatic impact on the B. vulgatus treated epithelial cells, with apparent alteration of tight-junction proteins, Zo-1 and Occludin (FIGS. 15d and 16). Imaging studies also demonstrated potential impacts on cell morphology and actin networks of the Caco-2 cells treated with B. vulgatus (FIG. 16). Analysis of the cell shape within monolayers showed a significant decrease in the circularity of the cells (P=0.0043), which could be restored through protease inhibition (FIG. 15e).

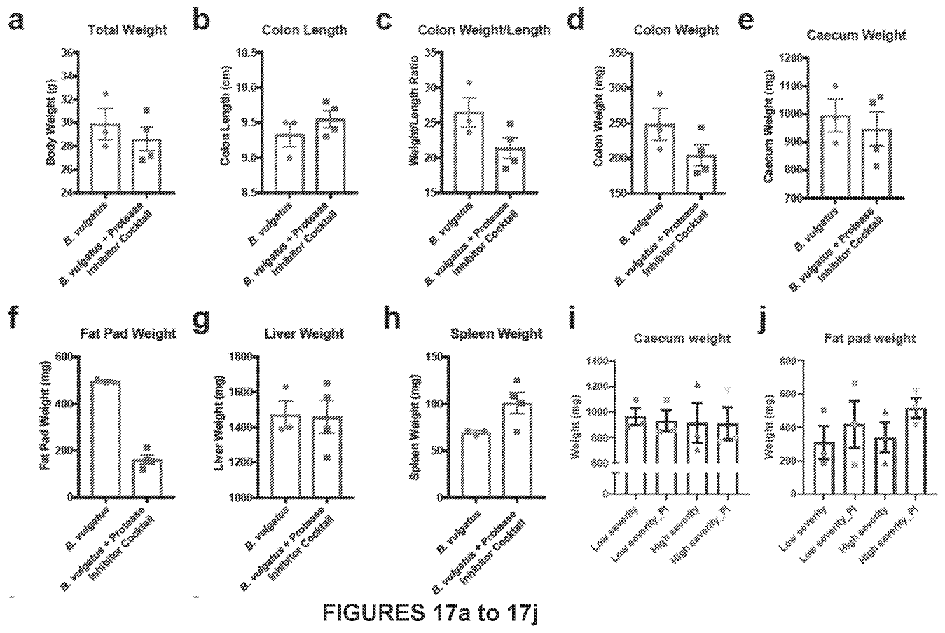

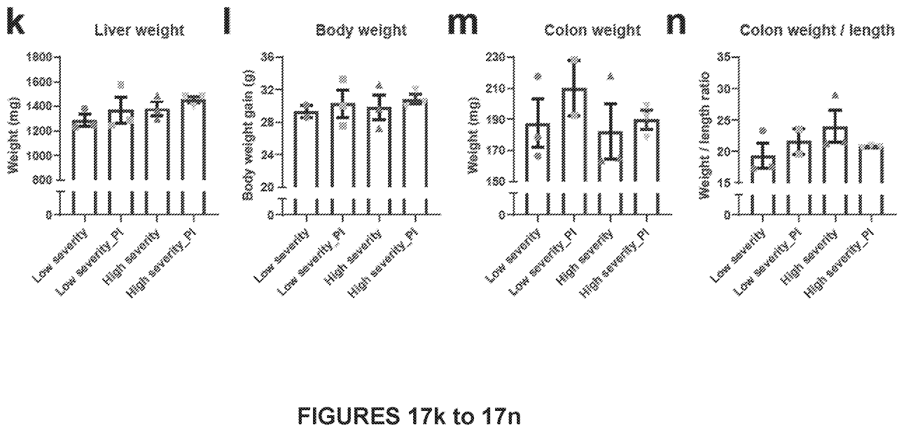

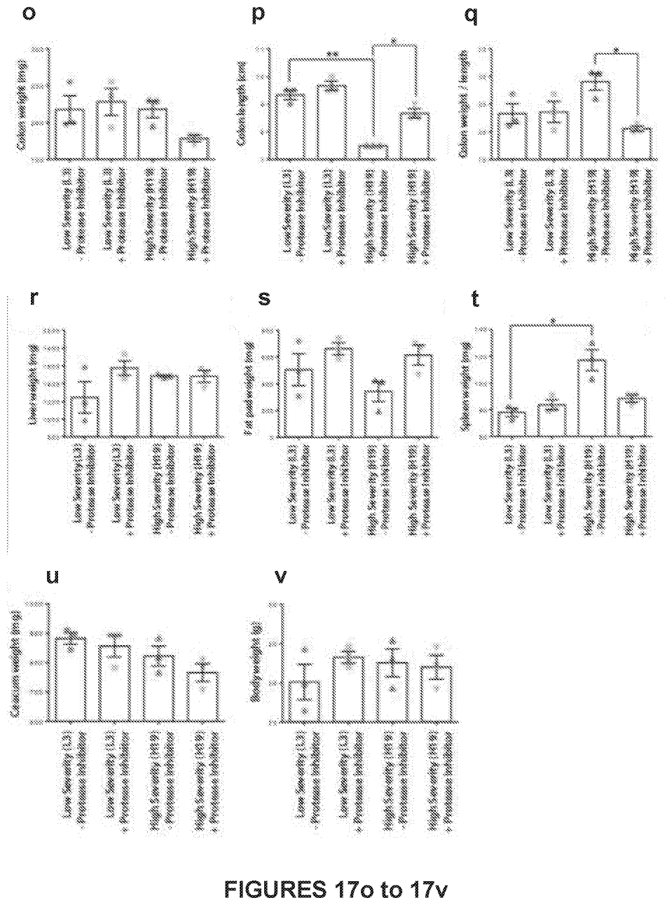

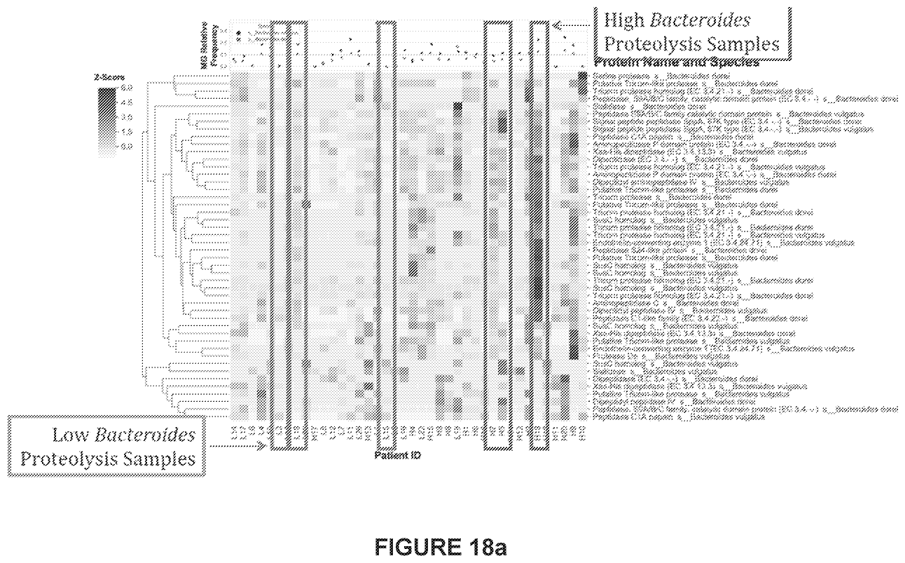

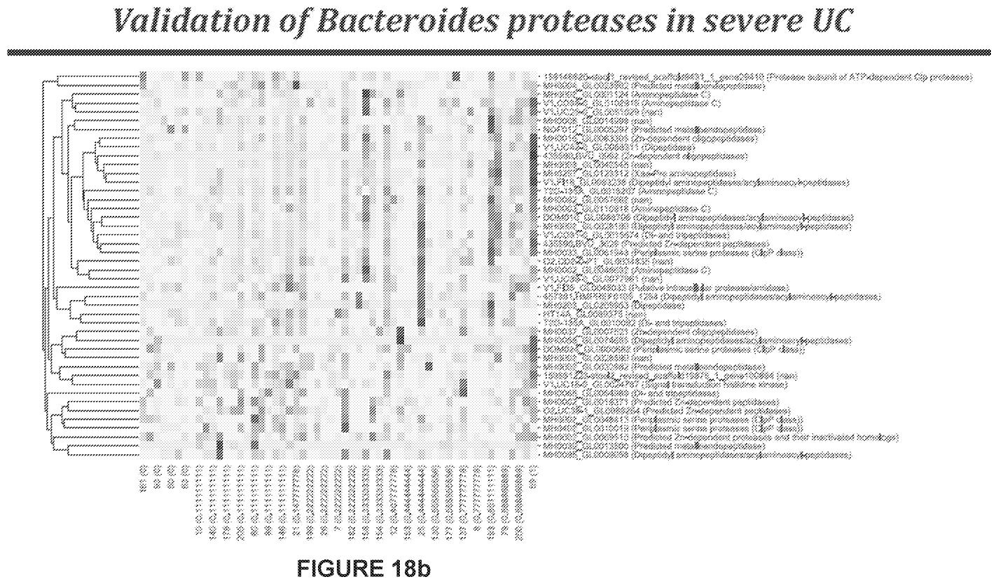

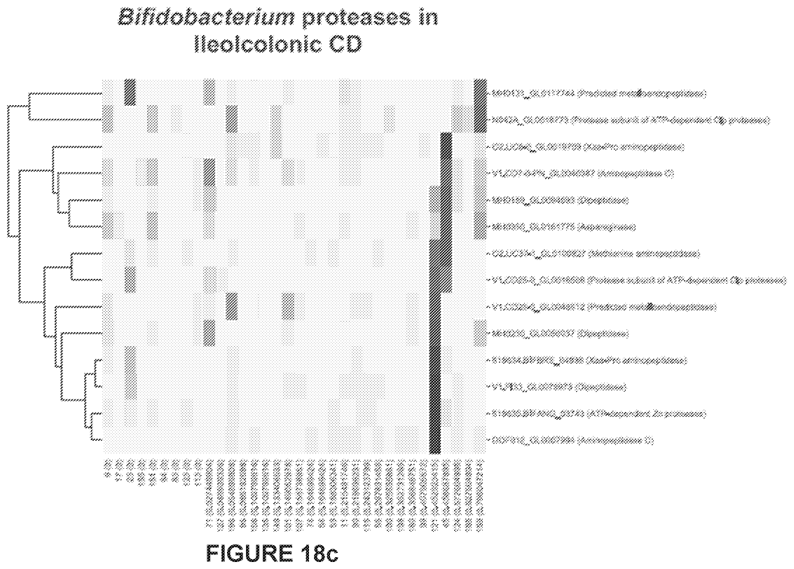

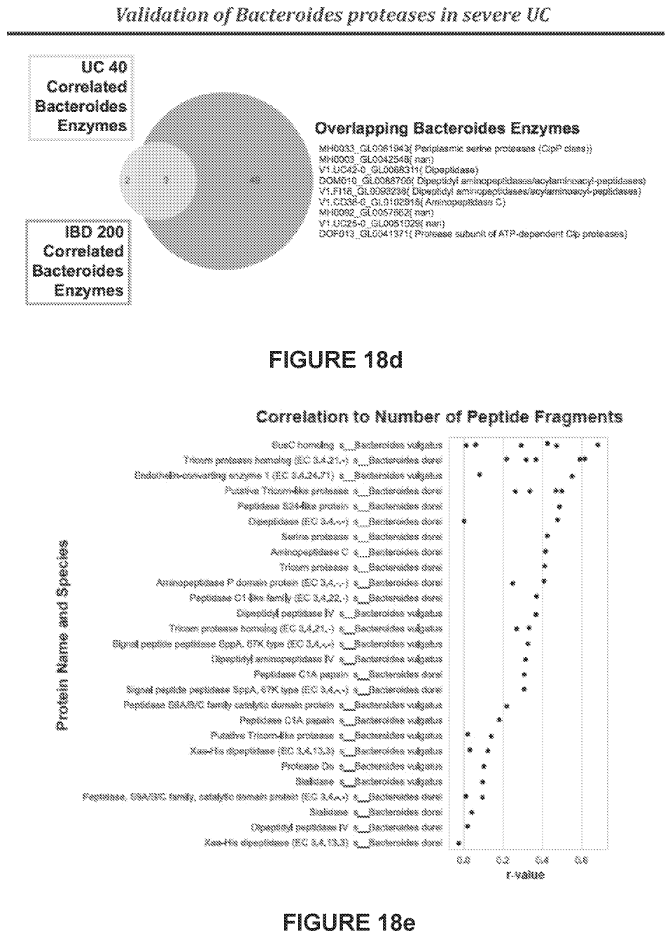

To investigate the effect of B. vulgatus proteases on colonic epithelia in vivo, Applicant next performed a monocolonization with B. vulgatus in an IL10^{-/-} germ-free mouse model (FIG. 15f). After 10-weeks of colonization, protease inhibition had a visible effect on the colonic epithelia (FIG. 15g). Histological analysis identified a trend for decreased histological colitis and crypt length through administration of the protease inhibitor cocktail (FIGS. 15h-15i). Whole organ measurements from monocolonized mice showed a significant reduction in the weight of the peri epididymal fat pad for the protease inhibitor group while other organs were not significantly changed (FIG. 17).

Protease Inhibition Prevents Colonic Inflammation from Patient Derived Fecal Transplants in Germ Free Mice

Next Applicant sought to evaluate the translational impact of protease inhibition for UC through utilizing fecal transplantation of patient samples into germ-free mice (FIG. 15j). For this experiment, 3 patient samples identified as having an over-abundance of B. vulgatus proteases were selected and compared to 3 patient samples with low B. vulgatus protease abundance. Through the duration of the study, mice were fed either water containing a protease inhibitor cocktail or water alone (n=3 per group), after which time mice were sacrificed and macroscopic measurements were taken (FIG. 15j). Protease abundant samples induced an average 20% reduction in colon length (FIG. 15k) and a 20% increase in spleen weight (FIG. 15l), without significant macroscopic impact on other organs (FIG. 17). Strikingly, the measurements most impacted by protease-abundant fecal samples significantly shifted toward the low severity groups in the protease inhibitor group (FIGS. 15k-15l). These studies reveal that the microbiome derived from UC patients who express high Bacteroides protease activity can induce pathological changes through protease activity, and that protease inhibition can have potential as a therapeutic intervention in severe UC.

Finally, to confirm the presence of Bacteroides vulgatus proteases in the fecal transplantation study, metaproteomic analysis of the mouse fecal material was performed. Comparing the fecal material of mice transplanted with samples from one patient with overabundant B. vulgatus proteases and one low protease control patient, Applicant were able to detect an increased abundance of B. vulgatus proteases from the overabundant transplantation irrespective of the presence of the protease inhibitor cocktail (FIG. 15m). Applicant have further confirmed that several of these proteases contain serine-protease activity. To guide future studies into B. vulgatus proteases, further comparisons have been made comparing the identity of proteases correlated to UC patient activity to proteases uniquely identified in B. vulgatus or B. dorei supernatant and proteases that were significantly increased within the fecal samples of mice displaying the colitis phenotype from transplantation of UC fecal material (FIG. 15l, Table 4). Of note, several dipeptidyl peptidases (e.g. DPPIV, DPPVII) were identified throughout the study, which have known roles in amino acid metabolism in nutrient limited areas [PMID: 28408952] and virulence in Porphymonas gingivalis²³, a bacterium that can cause periodontal disease.

### DISCUSSION

Here, Applicant effectively collected and translated one of the most comprehensive meta-omic profiles of IBD patients to date into a hypothesis of biological and therapeutic value. Through integrating fecal metaproteomics, metabolomics, 16S gene amplicon sequencing, shotgun metagenomic sequencing, metapeptidomics, and serum proteomics, in addition to in vitro and in vivo validation, Applicant demonstrated that certain members of the microbiome, such as Bacteroides vulgatus, can contribute to exacerbating UC disease activity through protease activity. Further, given the promise of the in vitro and in vivo experiments as disclosed herein, this study sets the stage for further investigation of Bacteroides protease inhibition as a therapeutic approach in UC.

To generate the hypothesis, Applicant utilized several innovative-omic advances that can be of broad interest. The core of the findings as disclosed herein stemmed from the previously developed integrated approach for comparing metagenomic and metaproteomic data¹⁵. This allowed the identification of discrepancies between the more traditionally collected metagenomic and Applicant's multiplexed metaproteomic approach. Given that previous high-profile IBD data sets that included metaproteomic data⁶ used methods that generated an order of magnitude more missing values (i.e. sparsity), Applicant had interest in further investigating findings unique to the metaproteome. One striking observation uniquely highlighted in the study as disclosed herein was that ˜50% of microbial proteins correlated to UC disease activity were derived from Bacteroides. While metapeptidomic data is rarely collected in microbiome studies, this data provided an important complementary tool for identifying that proteolysis, potentially derived from Bacteroides proteases, was correlated to UC activity. By integrating metagenomic data, Applicant provided a genomic context to the findings as disclosed herein and identified Bacteroides species of interest for in vitro studies. Other-omic profiles (serum proteomics, metabolomics, and 16S) further corroborated and contextualized the core hypothesis of Bacteroides derived proteolysis as a contributing factor to UC severity.

The findings as disclosed herein on Bacteroides were derived in the backdrop of several previously described observations. An early metaproteomic study identified Bacteroides proteins as markers of CD²⁴, although genomic approaches only occasionally implicate Bacteroides^{4,25}, and Bacteroides functional role in IBD was not well established²⁶. Bacteroides typically reside in the outer mucosal layer of the colon²⁷, and are described as decreased in IBD²⁸. There is some evidence that commensal Bacteroides species can induce colitis in mouse models²⁹, although they are typically beneficial for their role of digesting complex carbohydrates^{26,30}. The in vitro studies as disclosed herein found a disruptive phenotype for only Bacteroides vulgatus and Bacteroides dorei (which are close phylogenetic neighbors¹⁸), and further, this phenotype was only ameliorated through serine and cysteine protease inhibition. This level of specificity provides a shortlist of bacterial species and proteases of interest for future studies. Focusing on the proteases of these two species and then cross-referencing metagenomic species abundances, Applicant were able to identify a subset of inflamed patients whom had unusually high bacterial protease presence. These patients represent a new subset of ˜40% UC patients that might benefit from bacterial protease inhibition.

The role of proteases in Bacteroides remains an underexplored research area. Early studies indicated that their proteases can have effects on host enzymes and that B. vulgatus had higher proteases activity than other Bacteroides species³¹. It is possible that higher than genetically expected protease abundance could be the result of increased membrane vesicles, which are known to be abundant in proteases in Bacteroides³². Interestingly, extracellular vesicles were linked to IBD and Bacteroides proteins were reported as a majority contributor to bacterial extracellular proteins⁹. Regarding the regulation of Bacteroides proteases, the C10 family of proteases is regulated by oxygen levels in some Bacteroides species³³. Further investigation into the regulation of Bacteroides outermembrane vesicles and proteases might be important areas of research for better understanding the role of bacterial proteases in UC.

Extracellular matrix remodeling³⁴ and protease activity²⁰ are known molecular events in IBD, but current treatments are focused on targeting host inflammatory pathways³⁵. Work in this area has mostly focused on the contribution of host proteases, such as trypsin, which is decreased in IBD patients³⁶, or matrix metalloproteases which can degrade commonly used therapeutics^{37,38}. The role of bacterial proteases in IBD has been primarily a source of speculation^{20,39-41}. Some authors estimate that ˜27% of proteolysis in UC patients is from bacterial proteases⁴². Changes in gastrointestinal serine protease activity after antibiotic exposure in mice implicates the activity of microbiome-derived serine proteases⁴³. However, studies into the roles of specific Bacteroides proteases in general are limited beyond the characterization of a metalloprotease enterotoxin from Bacteroides fragilis⁴⁴. The phenotypes Applicant observed specific to Bacteroides vulgatus and the fecal transplant studies as disclosed herein suggest that these bacterial serine proteases can have important contributions to proteolysis in UC.

The multidimensional meta-omic integration shown here serves as a landmark study in comparative—omics and the development of hypotheses from large-scale data integration. Starting with broad-scale analysis and further refining Applicant's studies according to an observation of interest led to important findings within each dataset. The efficacy of protease inhibition in vitro and in vivo validates the utility of our approach. In total, Applicant's study opens new areas of investigation regarding the role of proteolysis in Bacteroides, and demonstrates that proteolysis from Bacteroides vulgatus can be relevant to UC pathology and treatment.

### Experimental Example No. 2—Methods

Patient Population and Clinical Diagnostics

Ulcerative colitis and Crohn's disease patients were selected from a convenience sampling biobank at the University of California at San Diego (UCSD: PI Dulai). In this biobank patients consent to longitudinal data collection on patient demographics (age, gender, ethnicity), disease characteristics (prior surgeries, disease-related complications, phenotype classification according to Montreal sub-classifications), current and prior treatments (corticosteroids, immunomodulators, biologics), clinical disease activity (patient reported outcomes using the partial Mayo score and Crohn's disease activity index), and endoscopic and histologic disease activity for ulcerative colitis patients. Alongside this data collection patients agree to stool, serum, and mucosal biopsy collection. When endoscopy was performed as part of routine practice, stool was collected within 24 hours prior to endoscopy and serum was collected the day of endoscopy. At each endoscopy, a physician with advanced training in IBD performed a detailed endoscopic disease activity assessment using the Mayo endoscopic sub-score and the Ulcerative Colitis Endoscopic Index of Severity (UCEIS), without knowledge of the clinical disease activity score or biomarker data. Routine standard of care biopsies were scored using the Geboes score by a pathologist with training and expertise in IBD, who was blinded to clinical, biomarker, and endoscopic data and scores. Further information regarding clinical, endoscopic and histologic activity scoring have been previously discussed¹⁴. All serum and stool samples were aliquoted within 24 hours of collection to avoid future freeze-thaw cycles, and samples were stored at −80° C.

DNA Extraction

Frozen samples were thawed and transferred into 96-well plates containing garnet beads and extracted using Qiagen MagAttract DNA kit adapted for magnetic bead purification as previously described⁴⁵. DNA was eluted in 100 μl Qiagen elution buffer.

16S Gene Amplicon Sequencing

16S rRNA gene amplicon sequencing was performed according to the Earth Microbiome Project. Briefly, the V4 region of the 16S rRNA gene (515f/806r) was amplified from 1 μl DNA per sample in triplicate^{46,47}. Amplicons were quantified with Quant-iT™ PicoGreen™ dsDNA Assay Kit, and 240 ng, or maximum 15 ul, of each sample was pooled into a final library and cleaned using the QIAquick PCR Purification Kit. Paired-end sequencing was performed on the Illumina MiSeq using MiSeq Reagent Kit v3 (300-cycle).

Shotgun Metagenomic Sequencing

Extracted DNA was quantified with PicoGreen™ dsDNA Assay Kit, and 1 ng of input, or maximum 3.5 μl, gDNA was used in a 1:10 miniaturized Kapa HyperPlus protocol. Per sample libraries were quantified and pooled at equal nanomolar concentration. The pooled library was cleaned with the QIAquick PCR Purification Kit and size selected for fragments between 300 and 700 bp on the Sage Science PippinHT. The pooled library was sequenced as a paired-end 150-cycle run on an Illumin HiSeq4000 v2 at the UCSD IGM Genomics Center.

Processing of Metagenomic Reads for a Shared Reference Library

Because typical metagenomics and metaproteomics workflows require a reference database, it was necessary to create from scratch using a single reference database that could be used for both metagenomics and metaproteomics from the individualized data as previously described¹⁵. Individual samples were first trimmed and host-filtered using trimmomatic[24695404] and bowtie2 [22388286]. All reads from each sample were concatenated. Next the tool MEGAHIT⁴⁸ was utilized for assembling short reads into larger contigs. Assembled contigs were searched for possible coding regions through the program Prodigal⁴⁹. Next, the program Diamond⁵⁰ was used for gene alignment to the uniref50 database. Finally, the most likely uniref50 entry, determined through bitScore, was used for the functional annotations. KEGG orthology annotations were cross-referenced using GhostKOALA⁵¹. Taxonomic assignments were determined by Diamond alignment⁵⁰ to an extensive database of bacterial and archaeal genomes⁵². This study-specific database was used as a reference database for both metaproteomic data and shotgun sequencing data. Scripts used for data processing are available online (github.com/knightlab-analyses/uc-severity-multiomics).

Unweighted UniFrac Analysis of Shotgun Metagenomic Data

Taxonomic profiling of shotgun sequences was performed using Centrifuge 1.0.3 with default parameter settings against the aforementioned in-house microbial genome database. The numbers of reads mapped to individual reference genomes per sample were summarized into a BIOM table. Genomes mapped by less than 0.01% reads per sample were dropped. The beta diversity of samples was assessed using the unweighted UniFrac metric as implemented in QIIME⁵³, with reference to the phylogenetic tree of the microbial genomes (also available at: github.com/biocore/wol). The resulting distance matrix was visualized with PCoA, and the hypothesis was tested using PERMANOVA and Adonis as implemented in QIIME⁵³.

Generating Copy Numbers of Metagenomic Genes

The program Salmon⁵⁴ was applied to determine the reads present for each gene from the shared reference library described above. First, an index was created with Salmon inputting the shared reference library's fasta file. Next, reads were aligned to this index in quasi-mapping mode for each of the metagenomic samples. The results were represented in counts per million sequences, with missing values padded as zeroes.

Serum Collection, Depletion and Analysis

Seppro human depletion kits were used according to manufacturer protocols for depletion of highly abundant proteins. After thawing samples on ice, 14 μL of serum was applied to columns following the depletion protocol, and the wash and elution fractions were combined to increase the total protein content. After depletion, protein was processed as described below, with the exception of a TCA precipitation⁵⁵ being used in place of chloroform methanol extraction. After data collection and processing, large variability was observed dependent on serum coloring, and 7 samples with study identifiers L7, L15, L13, L8, L18, L6 and H17 (which were colored red likely because of the presence of blood in the serum) were removed for PCoA visualization.

Protein Preparation

Fecal samples were measured out to ˜0.5 g and suspended in 5 mL of ice-cold, sterile TBS. Samples were vortexed until completely suspended. Two 20 μM vacuum, steriflip (Milipore) filters were used per sample to remove particulate. Cells were pelleted through centrifugation at 4000 rpm for 10 min at 4° C. Next, cells were lysed in 2 mL of buffer containing 75 mM NaCl (Sigma), 3% sodium dodecyl sulfate (SDS, Fisher), 1 mM NaF (Sigma), 1 mM beta-glycerophosphate (Sigma), 1 mM sodium orhtovanadate (Sigma), 10 mM sodium pyrophosphate (Sigma), 1 mM phenylmethylsulfonyl fluoride (PMSF, Sigma), and 1× Complete Mini EDTA-free protease inhibitors (Roche) in 50 mM HEPES (Sigma), pH 8.5⁵⁶. An equal volume of 8M Urea in 50 mM HEPES, pH 8.5 was added to each sample. Cell lysis was achieved through two 15-second intervals of probe sonication at 25% amplitude. Proteins were then reduced with dithiothreitol (DTT, Sigma), alkylated through iodoacetamide (Sigma), and quenched as previously described⁵⁷. Proteins were next precipitated via chloroform-methanol precipitation and protein pellets were dried⁵⁸. Protein pellets were re-suspended in 1M urea in 50 mM HEPES, pH 8.5 and digested overnight at room temperature with LysC (Wako)⁵⁹. A second, 6-hour digestion using trypsin at 37° C. was performed and the reaction was stopped through addition of 10% trifluoroacetic acid (TFA, Pierce). Samples were then desalted through C18 Sep-Paks (Waters) and eluted with a 40% and 80% Acetonitrile solution containing 0.5% Acetic Acid⁶⁰. Concentration of desalted peptides was determined with a BCA assay (Thermo Scientific). 50 μg aliquots of each sample were dried in a speed-vac. Additionally bridge channels consisting of 25 μg from each sample were created and a 50 μg aliquots of this solution were used in duplicate per Tandem Mass Tag (TMT) 10 plex MS experiment as previously described⁶¹. These bridge channels were used to control for labeling efficiency, inter-run variation, mixing errors and the heterogeneity present in each sample⁶². Each sample or bridge channel was resuspended in 30% dry acetonitrile in 200 mM HEPES, pH 8.5 for TMT labeling with 7 μL of the appropriate TMT reagent⁶³. Reagents 126 and 131 (Thermo Scientific) were used to bridge between mass spec runs. Remaining reagents were used to label samples in random order. Labeling was carried out for 1 hour at room temperature, and quenched by adding 8 μL of 5% hydroxylamine (Sigma). Labeled samples were acidified by adding 50 μL of 1% TFA. After TMT labeling each 10-plex experiment was combined and desalted through C18 Sep-Paks and dried in a speed-vac.

Generation and Processing of Proteomic Data Through LC-LC-MS²/MS³

Basic pH reverse-phase liquid chromatography (LC) followed by data acquisition through LC MS²/MS³ was performed as previously described⁶¹. Briefly, 60-minute linear gradients of acetonitrile were performed on C18 columns using an Ultimate 3000 HPLC (Thermo Scientific). Subsequently, 96 fractions were combined as previously described⁶⁴, and further separation of fractions was performed with an in-line Easy-nLC 1000 (Thermo Fisher Scientific) and a chilled autosampler. LC-MS²/MS³ data was collected on an Orbitrap Fusion (Thermo Fisher Scientific) mass spectrometer with acquisition and separation settings as previously defined⁶⁵.

Data was processed using Proteome Discoverer 2.1 (Thermo Fisher Scientific). MS² data was searched against the shared metagenomic database and Uniprot Human database (uniprot.org, accessed May 11, 2017). The Sequest searching algorithm⁶⁶ was used to align spectra to database peptides. A precursor mass tolerance of 50 parts per million (ppm)^{67,68} was specified and 0.6 Da tolerance for MS² fragments. Included in the search parameters was static modification of TMT 10-plex tags on lysine and peptide n-termini (+229.162932 Da), carbamidomethylation of cysteines (+57.02146 Da), and variable oxidation of methionine (+15.99492 Da). Raw data was searched at a peptide and protein false discovery rate of 1% using a reverse database search strategy^{69-71.}

TMT reporter ion intensities were extracted from MS³ spectra for quantitative analysis and signal-to-noise values were used for quantitation. Additional stringent filtering was used removing any moderate confidence peptide spectral matches (PSMs), or ambiguous PSM assignments. Additionally, any peptides with a spectral interference above 25% were removed, as well as any peptides with an average signal to noise ratio less than 10. As metaproteome data contains a high degree of homology between proteins, several decisions were made to reduce false assignments for the metaproteome dataset. The standardized methods in Proteome Discoverer (Version 2.1) preferentially assign peptides to proteins that previously had peptides reported. If this does not resolve the assignment, the peptide is assigned to the longest protein. After the first search, all proteins reported in forward or reverse datasets were filtered into a smaller database for a second search as previously described¹⁶. This method effectively decreased the search space from the database used in cohort 1 from 748 mb to 21.8 mb. Any PSMs assigned to proteins from the reverse databases were removed. Additionally, a duplicate peptide filter was performed according to the Proteome Discoverer report. Relative abundances were normalized first to the pooled standards for each protein and then to the median signal across the pooled standard. An average of these normalizations was used for the next step. To account for slight differences in amounts of protein labeled, these values were then normalized to the median of the entire dataset and reported as final normalized summed signal-to-noise ratios per protein per sample.

Metabolite Extraction and LC-MS²

Metabolites were extracted by adding a 1:5 weight to volume solution of 70% methanol infused with a 5 μM internal standard sulfamethoxine. The samples were briefly vortexed to mix and stored at 4° C. overnight. Extracts were then centrifuged at 4000 rpm for 5 minutes to pellet particulate matter and the supernatant was removed for MS analysis. The extracts were diluted 1:4 in a 96 well plate in pure methanol prior to injection.

LC-MS/MS was performed on a Bruker Daltonics® Maxis qTOF mass spectrometer (Bruker, Billerica, Mass. USA) with a ThermoScientific UltraMate 3000 Dionex UPLC (Fisher Scientific, Waltham, Mass. USA). Metabolites were separated using a Kinetex 2.6 μm C18 (30×2.10 mm) UPLC column with a guard column. Mobile phases were A 98:2 and B 2:98 ratio of water and acetonitrile containing 0.1% formic acid and a linear gradient from 0 to 100% for a total run time of 840 s at a flow rate of 0.5 mL min⁻¹ were used. The mass spectrometer was calibrated daily using Tuning Mix ES-TOF (Agilent Technologies) at a 3 mL min⁻¹ flow rate. For accurate mass measurements, lock mass internal calibration used a wick saturated with hexakis (1H,1H,3H-tetrafluoropropoxy) phosphazene ions (Synquest Laboratories, m/z 922.0098) located within the source. Full scan MS spectra (m/z 50-2000) were acquired in the qTOF and the top ten most intense ions in a particular scan were fragmented using collision induced dissociation at 35 eV for +1 ions and 25 eV for +2 ions in the collision cell. Data dependent automatic exclusion protocol was used so that an ion was fragmented when it was first detected, then twice more, but not again unless its intensity was 2.5× the first fragmentation. This exclusion method was cyclical, being restarted after every 30 seconds.

Metabolite Annotation

Data was converted to the .mzXML format using the Bruker Data Analysis software and uploaded to GNPS (gnps.ucsd.edu/) through the MassIVE server under ID MSV000082457. Molecular networking was performed as follows: precursor and fragment ion mass tolerance 0.03 Da, minimum cosine score of 0.65, minimum matched fragment ions of 4, and minimum cluster size of 2. GNPS library searching was performed with the same minimum matched peaks and cosine score. All library hits were inspected for quality with the mirror plot feature in GNPS. Area under the curve feature abundances were calculated to produce a metabolome buckettable with the mzMine software. Parameters were as follows: MS1 noise level of 5000 counts, MS2 noise level of 150 counts, m/z tolerance of 0.03 Da for chromatogram building with a minimum time span of 0.1 min, isobaric peaks were deconvoluted with a minimum height of 5000 counts and using the base-line cutoff algorithm, peaks were deisotoped with the same mass tolerance, a 0.1 min retention time tolerance and a maximum isotopic peak pattern of 4, peaks were aligned with the same mass tolerance and retention time tolerance and filtered for at least 3 peaks in a sample and gap filling was performed to produce the final buckettable for statistical analysis. Molecular class annotations were generated through an automated pipeline on GNPS (www.biorxiv.org/content/10.1101/2020.05.04.077636v1) utilizing Sirius⁷² and ClassyFire⁷³.

Generation of Metapeptidome Data

LC-MS/MS .mzXML formatted files were loaded into PEAKS Studio 8.5²¹ for de novo identification and searching against the Uniprot human protein database. De novo error tolerance parameters were used according to PEAKS default qTOF settings, 0.1 Da parent mass error tolerance, 0.1 Da fragment mass error tolerance. The search settings included no added restriction enzymes, variable dehydration, Acetylation (N-Term), Oxidation (M), and Ubiquitination. The max variable post translational modifications per peptide was set to 3. De novo sequences were filtered to keep only those with an average local confidence above 85%.

For human peptides, Label free quantification was run through PEAKS Studio 8.5²¹. A 1% FDR cutoff was used integrating peaks with a 20 ppm mass error tolerance and a 6 min retention time window. Peptides were searched against the human protein database (uniprot.org, accessed May 11, 2017) for identification. Quantification was normalized to the total ion chromatograph.

Meta-Omic Data Analysis

Data analysis was performed in python (version 3.5), and records of the code are available in corresponding Jupyter Notebooks for this project (github.com/knightlab-analyses/uc-severity-multiomics). Clinical data correlations were performed on UC cohort 1 using the package seaborn's (seaborn.pydata.org/) clustermap function. Community diversity analysis was performed usin QIIME 2⁷⁴ (Version 2019.1), through the “qiime diversity core-metrics” command. Statistical analysis of beta-diversity plots was performed using ADONIS for quantitative variables and PERMANOVA for categorical variables using QIIME 2. Composition plots for—omics data were plotted using the package matplotlib (matplotlib.org/).

16S fastq were split, demultiplexed, trimmed to 150 base pairs, demultiplexed and processed through deblur using QIITA⁷⁵ (Study ID 11549). A denovo phylogenetic tree was formed for 16S data using the reference hits through QIIME 2⁷⁴ (version 2018.4) commands “qiime alignment mafft”, “qiime alignment mask”, “qiime phylogeny fasttree” and “qiime phylogeny midpoint-root”. 16S alpha-diversity was generated using QIIME 2⁷⁴ (Version 2019.1) through the command “qiime diversity core-metrics phylogenetic”. Kruskal-Wallis significance tests for alpha diversity were performed in QIIME 2⁷⁴ (Version 2018.4) using the “qiime diversity alpha-group-significance” command. Linear regressions between alpha diversity scores and quantitative categories were performed using the linregress command from the python package scipy (www.scipy.org).

Random forest regressions were performed using QIIME 2⁷⁴ (Version 2018.11) using the sample-classifier regress-sample command. The test size was set to 0.1. Statistics and importance scores for each feature within the 100 independent analyses were compiled.

Linear regression of metagenome, metabolome, and metaproteome data to partial Mayo were also performed using the linregress command as above. Before performing regression, missing values from the metagenome and metabolome were padded with zeros. Given the nature of TMT-labeled proteomic data, where protein abundances are often normalized to account for differences in the number of peptides identified, missing values in regressions were ignored, and the percentage of missing values in each protein was calculated. When comparing metagenome and metaproteome data, metagenomic data was analyzed accounting for missing values in an identical manner to metaproteomic data where missing values were ignored and sparsity of the data was monitored. Composition of genes or proteins significantly correlated with disease activity (linregress r >0.3) were compared as previously¹⁵, where taxonomic or functional annotations of significantly correlated or anti-correlated genes or proteins were compared.

To identify patient samples containing an over-abundance of Bacteroides vulgatus proteases, an outlier approach was taken using R studio (v. 1.1.383) using the bagplot function from the aplpack package. After applying a BLASTp analysis (blast.ncbi.nlm.nih.gov/) to the peptide sequences identified in UC patient metaproteomic studies identified as being correlated to disease activity and derived from B. vulgatus or B. dorei proteases, Applicant determined that it could not specify the origin of these proteases beyond being derived from either B. vulgatus or B. dorei. As a result, outlier analysis was performed using the summed metaproteomic abundances of all correlated (r >0.3) proteases from B. vulgatus and B. dorei. These values were compared to the summed abundance of metagenomic reads mapped to B. vulgatus and B. dorei. Outliers identified above the best-fit line were classified as Bacteroides protease “overproducers” while outliers identified below the best-fit line were classified as “underproducers”. All other patient samples were categorized as “others”. Statistical comparisons of patient endoscopic and disease activity scores between these groups were performed using independent t-tests through the package scipy.

Host protein networks were compiled from serum and fecal proteomics data from UC cohort 1. Linregress correlation values (r) between proteins and disease activity (partial Mayo scores) were used to rank associations. Top ranked proteins were uploaded to STRING-db⁷⁶, with associations between proteins determined through default settings, accounting for textmining, experiments, databases, co expression, neighborhood, gene fusion and co-occurrence. Networks were next visualized through Cytoscape (version 3.5.1)⁷⁷.

The program iceLogo's web application⁷⁸ was used for consensus sequence analysis. The first and last amino acids from peptides with an average local confidence over 85% were analyzed against a background using the percentage scoring system. For metapeptidome consensus sequences, all residues from peptides with over 85% average local confidence were used as background. For human consensus sequences, the precompiled Homo sapiens Swiss-Prot database was used.

Bacterial Supernatant Protease Activity Studies

Overnight cultures of the ATCC-derived Bacteroides vulgatus strain were grown anaerobically in Brain-heart-infusion (BHI) broth supplemented with Vitamin K and Hemin. Supernatant was collected by pelleting cells at 8000 g. Supernatant was then 8-fold concentrated by application of 3,300 g for 15 minutes using 10 kDa Amicon Ultra-15 filters (Millipore). Concentrated supernatant was tested for protease activity using the EnzChek protease activity assay (Invitrogen) in 96-well black flat-bottom plates (Corning). Protease activity was measured after incubation for 24 hours at 37° C. measuring fluorescence at 485 nm for excitation and 530 nm for emission. Protease inhibitors were administered at 10% total volume and inhibition was calculated by comparison to vehicle control wells. Protease inhibitors tested included water-solubilized 4(2-Aminoethyl)benzenesulfonyl Fluoride (AEBSF, MP Biomedicals), water-solubilized E-64 (Sigma), DMSO-solubilized GM6001 (EMD Millipore), and DMSO-solubilized Pepstatin A (MP Biomedicals). After analysis of a preliminary dilution series, max inhibition was found for each protease inhibitor near the highest concentration allowed by solubility, and these concentrations were used for subsequent studies.

Bacterial Supernatant Proteomics

ATCC strains for Bacteroides vulgatus and Bacteroides thetaiotaomicron (B. theta) alongside Human Microbiome Project strain #717 Bacteroides dorei CL02T00C15 were grown in technical triplicate anaerobically in supplemented BHI broth. Supernatant was concentrated as described above, and prepared for TMT-mediated LC-LC-MS²/MS³ analysis as described above. Data was searched in Proteome Discoverer as described above using uniprot reference proteomes for each strain (www.uniprot.org, downloaded 8/24/2020). Code for the normalization and analysis of the bacterial supernatant proteomics data can be found in the github repository for this project (github.com/knightlab-analyses/uc-severity-multiomics).

Caco-2 Transwell Studies

Caco-2 cell transwell studies were conducted essentially as previously describer. Briefly, Caco-2 cells (passage number ranging from 14-30) were plated into collagen coated 6.5 mm inserts with 0.4 μm pores (Fisher Scientific). Cells were then cultured for 2.5 weeks prior to bacterial inoculation, changing media every 2 days. A day before inoculation, media was changed to media without antibiotics and when indicated, protease inhibitors were dissolved at a given concentration. TEER was measured prior to inoculation of bacteria, and measurements at each following timepoint referenced the original TEER measurement prior to inoculation. Transwell plates were allowed to equilibrate to room temperature for 20 minutes before each TEER timepoint. CFU estimates were performed through serial dilution of 10 μLs of media from inside of the transwell insert. Mammalian cell culture media consisted of DMEM with L-Glutamine (Corning) with 10% heat-inactivated fetal bovine serum, 100 μM sodium pyruvate (Corning), 0.75% sodium bicarbonate, 1× Insulin-Transferrin-Selenium (Gibco), 238.3 μM HEPES, and 1× Penicillin Streptomycin (Thermo). An antibiotic free version of the media consisted was used during bacterial inoculation containing the same contents with the exception of 2% heat-inactivated fetal bovine serum. A day prior to inoculation media was switched to the antibiotic free version, with or without protease inhibitors at the given concentrations. Protease inhibitors tested included Roche cOmplete EDTA-free protease inhibitor cocktail (Sigma).

Bacteroides strains derived from ATCC were used for vulgatus, fragilis, thetaiotaomicron, uniformis, and ovatus. Bacteroides dorei was derived from the Human Microbiome Project strain #717, Bacteroides dorei CL02T00C15. For inoculation, Bacteroides cultures were grown overnight in Brain heart-infusion (BHI) broth supplemented with Vitamin K and Hemin. Cultures were spun down at 8000 g, and resuspended in DMEM. Inoculations were performed through normalization by OD600 at an estimated multiplicity of infection of 5.

Confocal Microscopy

At the end point of transwell studies (38 hours post bacterial innoculation), cells were fixed and prepared for immunofluorescence as follows. Caco-2 cells were fixed on the transwell membrane at 37° C. for 10 minutes in 1 mL 4% Paraformaldehyde (Thermo) in PHEM (60 mM Piperzine-1,4-bis[2-ethanesulfonic Acid] Monosodium Salt, pH 6.9 [TCI Chemicals], 25 mM HEPES⁸⁰, 10 mM EGTA [Oakwood Chemical], 2 mM MgCl₂×6H₂O⁸⁰). Cells were next permeabilized for 5 minutes in PHEM with 0.5% Triton X-100 (Fisher) at room temperature. Next, 3×5-minute washes were performed in PHEM containing 0.1% Triton X-100 at room temperature. Cells were next blocked for 30 minutes in 1 mL AbDil (150 mM NaCl⁸⁰, 20 mM Tris-HCl, pH 7.4 [JT Baker], 0.1% Triton X-100⁸⁰, 2% Bovine serum albumin [Gemini Bioproducts]) at room temperature. After blocking, primary antibodies for Occludin (Thermo, catalog number 33-1500, 0.5 μg/mL) and ZO-1 (Thermo, catalog number 61-7300, 1.5 μg/mL) were added into AbDil and left in a humidified chamber overnight at 4° C. Cells were next washed 4× in PHEM containing 0.1% Triton X-100 for 5 minutes at room temperature. After washing, secondary antibodies, Rhodamine Red Donkey Anti-Rabbit (Jackson ImmunoResearch, Code Number 711-295-152), and Alexa Fluor 488 Donkey Anti-Mouse (Jackson ImmunoResearch) were diluted to 3 μg/mL in AbDil containing a 1:1000 dilution of Phalloidin-iFluor 647 (abcam, ab176759) and 1 μg/mL DAPI (Thermo). Secondary antibodies were incubated for 1 hour at room temperature in a humidified chamber. Following secondary antibody incubation, cells were again washed 3× in PHEM containing 0.1% Triton X-100 for 5 minutes at room temperature. Finally, cells were rinsed in PHEM, removed from transwell insert and fixed onto microscope slides for imaging.

Cells were imaged using a Nikon MR HD confocal with a four-line (405 nm, 488 nm, 561 nm, and 640 nm) LUN-V laser engine and DU4 detector using bandpass and longpass filters for each channel (450/50, 525/50, 595/50 and 700/75), mounted on a Nikon Ti2 using an Apo 60×1.49 NA objective, or a C2 Plus confocal with a similar four-line LUN-4 laser engine and a DUV-B detector operating in virtual bandpass mode. Images stacks were acquired with the galvo scanning mode on both confocals, and Z-steps of 0.2 μm. To avoid cross-talk between channels, Z-stacks were acquired of the DAPI and Rhodamine Red channels first, and the AlexaFluor 488 and Phalloidin-iFluor 647 channels were acquired subsequently. The laser powers used were 1.5% for the 405 nm laser 2% for the 488 nm laser, 1.5% for the 561 nm laser and 1.5% for the 640 nm laser.

The morphology of cells was analyzed in representative images using protocols outlined previously [PMID: 32658197]. Images were processed in ImageJ (imagej.nih.gov/ij/) using the MorpholibJ plugin [PMID: 27412086]. In brief, images were converted to binary, image borders were extended, and morphological segmentation was performed. Images were next outlined, dilated and analyzed for particles. Circularity values were plotted and significance between groups was assessed using independent t-tests through the package scipy.

Bacteroides vulgatus Monocolonized Mouse Studies

Germ-free C57BL/6 IL10^{-/-} male mice (C57BL/6NTac-Il10^{em8Tac}; Taconic model GF-16006) were maintained in isolated ventilated cages Isocages (Techniplast, West Chester, Pa., USA)⁸⁴. At 5-6 weeks of age, mice were orally administered with B. vulgatus. Two cages of mice were used (n=3-4 per cage), one with and one without a 1× concentration of Roche cOmplete EDTA free protease inhibitor cocktail (Sigma) administered in the drinking water of the mice throughout the colonization. After 10-weeks colonization, mice were then weighed, euthanized, and colon length, colon weight, spleen weight, liver weight, adipose weight and ceacum weight were measured. Small intestine and colon segments were fixed for histopathology scoring and analysis.

Gnotobiotic Mouse Fecal Transplant Studies

Germ-free C57BL/6 IL10^{-/-} male mice (C57BL/6NTac-Il10^{em8Tac}; Taconic model GF-16006) were maintained in isolated ventilated cages Isocages (Techniplast, West Chester, Pa., USA)⁸⁴. At 5-6 weeks of age, mice were orally administered with 200 μL of fecal suspension from three patients with overabundant B. vulgatus proteases (sample identifiers H5, H7 and H19) and three patients without overabundant B. vulgatus proteases (sample identifiers L3, L15 and L19). Transplanted mice were housed in isolated ventilated cages, Isocages and fed autoclaved Purina Rodent Chow #5021. Each sample was administered to two cages of mice (n=2-3 per cage), one with and one without a 1× concentration of Roche cOmplete EDTA-free protease inhibitor cocktail (Sigma) administered in the drinking water of the mice throughout the colonization. Mice were housed at Georgia State University (Atlanta, Ga., USA) and INSERM (Paris, France) under institutionally approved protocols (IACUC #A18006). Mice were then weighted, euthanized, and colon length, colon weight, spleen weight, liver weight, adipose weight and ceacum weight were measured.

Before the mice were euthanized, fecal samples were collected and snap-frozen for further analysis. Metaproteomic sample preparation and data acquisition was performed as described above for TMT-mediated LC-LC-MS²/MS³ analysis of fecal samples from cages associated with samples H19 and L3. For database search of mass spectra, a custom database was generated using the metagenomic database generation workflow described above on metagenomic sequencing data from the specific UC patient donors. Here, to help track the origin of protein sequences, H19 and L3 reads were assembled and searched for coding regions separately. Open reading frames from each patient sample were combined and annotated as described above. Spectra were searched in Proteome Discoverer against this database alongside the uniprot mouse reference database. Code records for normalization and analysis of this data are available on github (github.com/knight-labanalyses/uc-severity-multiomics).

### Data Availability

Metabolomic data, Proteomic data and supplementary files are available online at massive.ucsd.edu (study ID MSV000082094). Genomic data is being uploaded through EBI www.ebi.ac.uk/ena.

### Experimental Discussion

Applicant analyzed fecal and serum samples from 40 patients with Ulcerative Colitis (UC) by performing a series of genomics, metabolomics, metapeptidomics, and metaproteomics analyses, Applicant found that Bacteroides vulgatus, was enriched in the gut of these UC patients. Bacteroides vulgatus disrupted intestinal epithelial permeability in vitro, but protease inhibition was sufficient to restore the epithelial barrier. When fecal material from UC patients were transplanted into germ-free mice, these mice ended up with increased colitis. However, oral administration of protease inhibitors attenuated disease severity. This means that targeting microbial proteases can ameliorate the intestinal barrier dysfunction and restore mucosal integrity.

In another aspect, Applicant acquired new data on a very large-scale validation cohort including over 200 patients, which contained 70 new ulcerative colitis patients, nearly 100 Crohn's disease controls with novel insights into bacterial proteases by disease sub-type (ileal, ileocolonic, isolated colonic), and a healthy control cohort. Indeed, the results with this cohort corroborate Bacteroides proteases as a factor unique to ulcerative colitis severity, with alternative bacterial proteases driving disease activity in Crohn's disease. Technically, the proteome analysis led to an unprecedented identification of over 80,000 fecal proteins from both host and microbial origin. In addition, Applicant performed several additional in vitro experiments specifying that serine proteases from Bacteroides vulgatus are relevant to disease activity.

### EQUIVALENTS

Unless otherwise defined, all technical and scientific terms used herein have the same meaning as commonly understood by one of ordinary skill in the art to which this invention belongs.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that the materials, methods, and examples provided here are representative of preferred embodiments, are exemplary, and are not intended as limitations on the scope of the invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

### REFERENCES

1. Fumery, M. et al. Natural History of Adult Ulcerative Colitis in Population-based Cohorts: A Systematic Review. Clin Gastroenterol Hepatol 16, 343-356 e343, doi:10.1016/j.cgh.2017.06.016 (2018).
2. Sartor, R. B. & Wu, G. D. Roles for Intestinal Bacteria, Viruses, and Fungi in Pathogenesis of Inflammatory Bowel Diseases and Therapeutic Approaches. Gastroenterology 152, 327-339 e324, doi:10.1053/j.gastro.2016.10.012 (2017).
3. Dulai, P. S., Siegel, C. A., Colombel, J. F., Sandborn, W. J. & Peyrin-Biroulet, L. Systematic review: Monotherapy with antitumour necrosis factor alpha agents versus combination therapy with an immunosuppressive for IBD. Gut 63, 1843-1853, doi:10.1136/gutjnl-2014-307126 (2014).
4. Schirmer, M. et al. Compositional and Temporal Changes in the Gut Microbiome of Pediatric Ulcerative Colitis Patients Are Linked to Disease Course. Cell Host Microbe 24, 600-610 e604, doi:10.1016/j.chom.2018.09.009 (2018).
5. Shen, Z. H. et al. Relationship between intestinal microbiota and ulcerative colitis: Mechanisms and clinical application of probiotics and fecal microbiota transplantation. World J Gastroentero 24, 5-14, doi:10.3748/wjg.v24.i1.5 (2018).
6. Lloyd-Price, J. et al. Multi-omics of the gut microbial ecosystem in inflammatory bowel diseases. Nature 569, 655-662, doi:10.1038/s41586-019-1237-9 (2019).
7. Verberkmoes, N. C. et al. Shotgun metaproteomics of the human distal gut microbiota. ISME J 3, 179-189, doi:10.1038/ismej.2008.108 (2009).
8. Jansson, J. K. & Baker, E. S. A multi-omic future for microbiome studies. Nat Microbiol 1, 16049, doi:10.1038/nmicrobiol.2016.49 (2016).
9. Zhang, X. et al. Metaproteomics reveals associations between microbiome and intestinal extracellular vesicle proteins in pediatric inflammatory bowel disease. Nature Communications 9, doi:ARTN 2873 10.1038/s41467-018-05357-4 (2018).
10. Franzosa, E. A. et al. Gut microbiome structure and metabolic activity in inflammatory bowel disease. Nat Microbiol, doi:10.1038/s41564-018-0306-4 (2018).
11. Erickson, A. R. et al. Integrated metagenomics/metaproteomics reveals human host-microbiota signatures of Crohn's disease. PLoS One 7, e49138, doi:10.1371/journal.pone.0049138 (2012).
12. Lewis, J. D. et al. Use of the noninvasive components of the Mayo score to assess clinical response in ulcerative colitis. Inflamm Bowel Dis 14, 1660-1666, doi:10.1002/ibd.20520 (2008).
13. Narula, N., Alshahrani, A. A., Yuan, Y., Reinisch, W. & Colombel, J. F. Patient-Reported Outcomes and Endoscopic Appearance of Ulcerative Colitis: A Systematic Review and Meta-Analysis. Clin Gastroenterol Hepatol, doi:10.1016/j.cgh.2018.06.015 (2018).
14. Dulai, P. S., Levesque, B. G., Feagan, B. G., D'Haens, G. & Sandborn, W. J. Assessment of mucosal healing in inflammatory bowel disease: review. Gastrointest Endosc 82, 246-255, doi:10.1016/j.gie.2015.03.1974 (2015).
15. Mills, R. H. et al. Evaluating Metagenomic Prediction of the Metaproteome in a 4.5-Year Study of a Patient with Crohn's Disease. mSystems 4, e00337-00318, doi:10.1128/mSystems.00337-18 (2019).
16. Zhang, X. et al. MetaPro-IQ: a universal metaproteomic approach to studying human and mouse gut microbiota. Microbiome 4, doi:Artn 31 10.1186/S40168-016-0176-Z (2016).
17. Li, J. et al. An integrated catalog of reference genes in the human gut microbiome. Nat Biotechnol 32, 834-841, doi:10.1038/nbt.2942 (2014).
18. Bakir, M. A., Sakamoto, M., Kitahara, M., Matsumoto, M. & Benno, Y. Bacteroides dorei sp. nov., isolated from human faeces. Int J Syst Evol Microbiol 56, 1639-1643, doi:10.1099/ijs.0.64257-0 (2006).
19. Kulagina, E. V. et al. Species Composition of Bacteroidales Order Bacteria in the Feces of Healthy People of Various Ages. Biosci Biotech Bioch 76, 169-171, doi:10.1271/bbb.110434 (2012).
20. Vergnolle, N. Protease inhibition as new therapeutic strategy for GI diseases. Gut 65, 1215-1224, doi:10.1136/gutjnl-2015-309147 (2016).
21. Zhang, J. et al. PEAKS DB: de novo sequencing assisted database search for sensitive and accurate peptide identification. Mol Cell Proteomics 11, M111 010587, doi:10.1074/mcp.M111.010587 (2012).
22. O'Donoghue, A. J. et al. Global substrate profiling of proteases in human neutrophil extracellular traps reveals consensus motif predominantly contributed by elastase. PLoS One 8, e75141, doi:10.1371/journal.pone.0075141 (2013).
23. Kumagai, Y. et al. Enzymatic properties of dipeptidyl aminopeptidase IV produced by the periodontal pathogen Porphyromonas gingivalis and its participation in virulence. Infect Immun 68, 716-724 (2000).
24. Juste, C. et al. Bacterial protein signals are associated with Crohn's disease. Gut 63, 1566-1577, doi:10.1136/gutjnl-2012-303786 (2014).
25. Vich Vila, A. et al. Gut microbiota composition and functional changes in inflammatory bowel disease and irritable bowel syndrome. Sci Transl Med 10, doi:10.1126/scitranslmed.aap8914 (2018).
26. Wexler, H. M. Bacteroides: the good, the bad, and the nitty-gritty. Clin Microbiol Rev 20, 593-621, doi:10.1128/CMR.00008-07 (2007).
27. Donaldson, G. P., Lee, S. M. & Mazmanian, S. K. Gut biogeography of the bacterial microbiota. Nat Rev Microbiol 14, 20-32, doi:10.1038/nrmicro3552 (2016).
28. Zhou, Y. & Zhi, F. Lower Level of Bacteroides in the Gut Microbiota Is Associated with Inflammatory Bowel Disease: A Meta-Analysis. Biomed Res Int 2016, 5828959, doi:10.1155/2016/5828959 (2016).
29. Bloom, S. M. et al. Commensal Bacteroides species induce colitis in host-genotype-specific fashion in a mouse model of inflammatory bowel disease. Cell Host Microbe 9, 390-403, doi:10.1016/j.chom.2011.04.009 (2011).
30. Foley, M. H., Cockburn, D. W. & Koropatkin, N. M. The Sus operon: a model system for starch uptake by the human gut Bacteroidetes. Cell Mot Life Sci 73, 2603-2617, doi:10.1007/s00018-016-2242-x (2016).
31. Riepe, S. P., Goldstein, J. & Alpers, D. H. Effect of secreted Bacteroides proteases on human intestinal brush border hydrolases. J Clin Invest 66, 314-322, doi:10.1172/JCI109859 (1980).
32. Elhenawy, W., Debelyy, M. O. & Feldman, M. F. Preferential packing of acidic glycosidases and proteases into Bacteroides outer membrane vesicles. MBio 5, e00909-00914, doi:10.1128/mBio.00909-14 (2014).
33. Thornton, R. F., Murphy, E. C., Kagawa, T. F., O'Toole, P. W. & Cooney, J. C. The effect of environmental conditions on expression of Bacteroides fragilis and Bacteroides thetaiotaomicron C10 protease genes. BMC Microbiol 12, 190, doi:10.1186/1471-2180-12-190 (2012).
34. Shimshoni, E., Yablecovitch, D., Baram, L., Dotan, I. & Sagi, I. ECM remodelling in IBD: innocent bystander or partner in crime? The emerging role of extracellular molecular events in sustaining intestinal inflammation. Gut 64, 367-372, doi:10.1136/gutjnl-2014-308048 (2015).
35. Ordas, I., Eckmann, L., Talamini, M., Baumgart, D. C. & Sandborn, W. J. Ulcerative colitis. Lancet 380, 1606-1619, doi:10.1016/S0140-6736(12)60150-0 (2012).
36. Denadai-Souza, A. et al. Functional Proteomic Profiling of Secreted Serine Proteases in Health and Inflammatory Bowel Disease. Sci Rep 8, 7834, doi:10.1038/s41598-018-26282-y (2018).
37. O'Sullivan, S., Gilmer, J. F. & Medina, C. Matrix metalloproteinases in inflammatory bowel disease: an update. Mediators Inflamm 2015, 964131, doi:10.1155/2015/964131 (2015).
38. Biancheri, P. et al. Proteolytic cleavage and loss of function of biologic agents that neutralize tumor necrosis factor in the mucosa of patients with inflammatory bowel disease. Gastroenterology 149, 1564-1574 e1563, doi:10.1053/j.gastro.2015.07.002 (2015).
39. Van Spaendonk, H. et al. Regulation of intestinal permeability: The role of proteases. World J Gastroenterol 23, 2106-2123, doi:10.3748/wjg.v23.i12.2106 (2017).
40. Steck, N., Mueller, K., Schemann, M. & Haller, D. Bacterial proteases in IBD and IBS. Gut 61, 1610-1618, doi:10.1136/gutjnl-2011-300775 (2012).
41. Carroll, I. M. & Maharshak, N. Enteric bacterial proteases in inflammatory bowel disease pathophysiology and clinical implications. World J Gastroenterol 19, 7531-7543, doi:10.3748/wjg.v19.i43.7531 (2013).
42. Gordon, M. H. et al. N-Terminomics/TAILS Profiling of Proteases and Their Substrates in Ulcerative Colitis. Acs Chem Biol 14, 2471-2483, doi:10.1021/acschembio.9b00608 (2019).
43. Roka, R. et al. Colonic luminal proteases activate colonocyte proteinase-activated receptor-2 and regulate paracellular permeability in mice. Neurogastroenterol Motil 19, 57-65, doi:10.1111/j.1365-2982.2006.00851.x (2007).
44. Obiso, R. J., Jr., Lyerly, D. M., Van Tassell, R. L. & Wilkins, T. D. Proteolytic activity of the Bacteroides fragilis enterotoxin causes fluid secretion and intestinal damage in vivo. Infect Immun 63, 3820-3826 (1995).
45. Marotz, C. et al. DNA extraction for streamlined metagenomics of diverse environmental samples. Biotechniques 62, 290-293, doi:10.2144/000114559 (2017).
46. Caporaso, J. G. et al. Ultra-high-throughput microbial community analysis on the Illumina HiSeq and MiSeq platforms. ISME J 6, 1621-1624, doi:10.1038/ismej.2012.8 (2012).
47. Thompson, L. R. et al. A communal catalogue reveals Earth's multiscale microbial diversity. Nature 551, 457-463, doi:10.1038/nature24621 (2017).
48. Li, D. H., Liu, C. M., Luo, R. B., Sadakane, K. & Lam, T. W. MEGAHIT: an ultra-fast single-node solution for large and complex metagenomics assembly via succinct de Bruijn graph. Bioinformatics 31, 1674-1676, doi:10.1093/bioinformatics/btv033 (2015).
49. Hyatt, D. et al. Prodigal: prokaryotic gene recognition and translation initiation site identification. BMC Bioinformatics 11, 119, doi:10.1186/1471-2105-11-119 (2010).
50. Buchfink, B., Xie, C. & Huson, D. H. Fast and sensitive protein alignment using DIAMOND. Nat Methods 12, 59-60, doi:10.1038/nmeth.3176 (2015).
51. Kanehisa, M., Sato, Y. & Morishima, K. BlastKOALA and GhostKOALA: KEGG Tools for Functional Characterization of Genome and Metagenome Sequences. J Mot Biol 428, 726-731, doi:10.1016/j.jmb.2015.11.006 (2016).
52. Zhu, Q. et al. Phylogenomics of 10,575 genomes reveals evolutionary proximity between domains Bacteria and Archaea. Nat Commun 10, 5477, doi:10.1038/s41467-019-13443-4 (2019).
53. Caporaso, J. G. et al. QIIME allows analysis of high-throughput community sequencing data. Nature Methods 7, 335-336, doi:10.1038/nmeth.f.303 (2010).
54. Patro, R., Duggal, G., Love, M. I., Irizarry, R. A. & Kingsford, C. Salmon provides fast and bias aware quantification of transcript expression. Nat Methods 14, 417-419, doi:10.1038/nmeth.4197 (2017).
55. Koontz, L. TCA precipitation. Methods Enzymol 541, 3-10, doi:10.1016/B978-0-12-420119-4.00001-X (2014).
56. Villen, J. & Gygi, S. P. The SCX/IMAC enrichment approach for global phosphorylation analysis by mass spectrometry. Nat Protoc 3, 1630-1638, doi:10.1038/nprot.2008.150 (2008).
57. Haas, W. et al. Optimization and use of peptide mass measurement accuracy in shotgun proteomics. Mol Cell Proteomics 5, 1326-1337, doi:10.1074/mcp.M500339-MCP200 (2006).
58. Wessel, D. & Flugge, U. I. A method for the quantitative recovery of protein in dilute solution in the presence of detergents and lipids. Anal Biochem 138, 141-143 (1984).
59. Van Rechem, C. et al. Lysine Demethylase KDM4A Associates with Translation Machinery and Regulates Protein Synthesis. Cancer Discov 5, 255-263, doi:10.1158/2159-8290.Cd-14-1326 (2015).
60. Tolonen, A. C. & Haas, W. Quantitative proteomics using reductive dimethylation for stable isotope labeling. J Vis Exp, doi:10.3791/51416 (2014).
61. Lapek, J. D., Jr. et al. Defining Host Responses during Systemic Bacterial Infection through Construction of a Murine Organ Proteome Atlas. Cell Syst, doi:10.1016/j.cels.2018.04.010 (2018).
62. Tolonen, A. C. et al. Proteome-wide systems analysis of a cellulosic biofuel-producing microbe. Mol Syst Biol 7, 461, doi:10.1038/msb.2010.116 (2011).
63. Thompson, A. et al. Tandem mass tags: a novel quantification strategy for comparative analysis of complex protein mixtures by MS/MS. Anal Chem 75, 1895-1904 (2003).
64. Wang, Y. et al. Reversed-phase chromatography with multiple fraction concatenation strategy for proteome profiling of human MCF10A cells. Proteomics 11, 2019-2026, doi:10.1002/pmic.201000722 (2011).
65. Lapek, J. D., Jr., Lewinski, M. K., Wozniak, J. M., Guatelli, J. & Gonzalez, D. J. Quantitative Temporal Viromics of an Inducible HIV-1 Model Yields Insight to Global Host Targets and Phospho-Dynamics Associated with Vpr. Mol Cell Proteomics, doi:10.1074/mcp.M116.066019 (2017).
66. Eng, J. K., McCormack, A. L. & Yates, J. R. An approach to correlate tandem mass spectral data of peptides with amino acid sequences in a protein database. J Am Soc Mass Spectrom 5, 976-989, doi:10.1016/1044-0305(94)80016-2 (1994).
67. Beausoleil, S. A., Villen, J., Gerber, S. A., Rush, J. & Gygi, S. P. A probability-based approach for high-throughput protein phosphorylation analysis and site localization. Nat Biotechnol 24, 1285-1292, doi:10.1038/nbt1240 (2006).
68. Huttlin, E. L. et al. A tissue-specific atlas of mouse protein phosphorylation and expression. Cell 143, 1174-1189, doi:10.1016/j.cell.2010.12.001 (2010).
69. Elias, J. E. & Gygi, S. P. Target-decoy search strategy for increased confidence in large-scale protein identifications by mass spectrometry. Nature Methods 4, 207-214, doi:10.1038/nmeth1019 (2007).
70. Elias, J. E., Haas, W., Faherty, B. K. & Gygi, S. P. Comparative evaluation of mass spectrometry platforms used in large-scale proteomics investigations. Nat Methods 2, 667-675, doi:10.1038/nmeth785 (2005).
71. Peng, J., Elias, J. E., Thoreen, C. C., Licklider, L. J. & Gygi, S. P. Evaluation of multidimensional chromatography coupled with tandem mass spectrometry (LC/LC-MS/MS) for large-scale protein analysis: the yeast proteome. J Proteome Res 2, 43-50 (2003).
72. Duhrkop, K., Shen, H., Meusel, M., Rousu, J. & Bocker, S. Searching molecular structure databases with tandem mass spectra using CSI:FingerID. Proc Natl Acad Sci USA 112, 12580-12585, doi:10.1073/pnas.1509788112 (2015).
73. Djoumbou Feunang, Y. et al. ClassyFire: automated chemical classification with a comprehensive, computable taxonomy. J Cheminform 8, 61, doi:10.1186/s13321-016-0174-y (2016).
74. Bolyen, E. et al. Reproducible, interactive, scalable and extensible microbiome data science using QIIME 2. Nat Biotechnol 37, 852-857, doi:10.1038/s41587-019-0209-9 (2019).
75. Gonzalez, A. et al. Qiita: rapid, web-enabled microbiome meta-analysis. Nat Methods 15, 796-798, doi:10.1038/s41592-018-0141-9 (2018).
76. Szklarczyk, D. et al. STRING v10: protein-protein interaction networks, integrated over the tree of life. Nucleic Acids Res 43, D447-452, doi:10.1093/nar/gku1003 (2015).
77. Shannon, P. et al. Cytoscape: a software environment for integrated models of biomolecular interaction networks. Genome Res 13, 2498-2504, doi:10.1101/gr.1239303 (2003).
78. Colaert, N., Helsens, K., Martens, L., Vandekerckhove, J. & Gevaert, K. Improved visualization of protein consensus sequences by iceLogo. Nat Methods 6, 786-787, doi:10.1038/nmeth1109-786 (2009).
79. Wang, F. et al. Interferon-gamma and tumor necrosis factor-alpha synergize to induce intestinal epithelial barrier dysfunction by up-regulating myosin light chain kinase expression. Am J Pathol 166, 409-419, doi:10.1016/s0002-9440(10)62264-x (2005).
80. Tremelling, M. et al. IL23R variation determines susceptibility but not disease phenotype in inflammatory bowel disease. Gastroenterology 132, 1657-1664, doi:10.1053/j.gastro.2007.02.051 (2007).
81. Costello S P et al. Effect of Fecal Microbiota Transplantation on 8-Week Remission in Patients With Ulcerative Colitis: A Randomized Clinical Trial. JAMA. 2019 Jan. 15; 321(2):156-164.
82. Moayyedi P et al. Fecal Microbiota Transplantation Induces Remission in Patients With Active Ulcerative Colitis in a Randomized Controlled Trial. Gastroenterology. 2015 July; 149(1):102-109.e6.
83. Paramsothy S et al. Specific Bacteria and Metabolites Associated With Response to Fecal Microbiota Transplantation in Patients With Ulcerative Colitis. Gastroenterology. 2019 April; 156(5): 1440-1454.e2.
84. Hecht G et al. A simple cage-autonomous method for the maintenance of the barrier status of germ-free mice during experimentation. Lab Anim. 2014 October; 48(4):292-7. Epub 2014 Aug. 5.
85. Koga Y et al. Exosome can prevent RNase from degrading microRNA in feces. J Gastrointest Oncol. 2011 December; 2(4): 215-222.

1. A method for treating or preventing one or more of: inflammatory bowel disease (MD), ulcerative colitis (UC), Crohn's disease (CD), comprising administering to a subject in need thereof an effective amount of a protease inhibitor that targets a protease expressed by a pathogenic bacterium.

2. The method of claim 1, wherein the CD is selected from a colonic CD, ileal CD, ileocolonic CD, or wherein the pathological bacteria comprises a Bacteroides bacteria and/or a bacteria as listed in FIGS. 5a to 5f, 6b, 6d 7a, 7b, 7d, 8a-8e, 9a, 9b, 14a, and Ism and/or Table 3.

3. (canceled)

4. The method of claim 2, wherein the protease is selected from one or more of a serine-protease, a metalloproteinase, an aspartyl protease and a cysteine-protease, and/or those identified in any one of FIGS. 7e, lie, Ilf, llg, and/or 18a-18e and/or Table 4, and/or wherein the inhibitor is selected from one or more of: AEBSF, E-64, GM6001, Roche cOmplete EDTA-free protease inhibitor cocktail or Pepstatin A.

5. The method of claim 4, wherein the Bacteroides is one or more of a Bacteroides identified in FIG. 7d, optionally one or more of a Bacteroides vulgatus Bacteroides dorei, Bacteroides theta or Bacteroides uniformis.

6. The method of claim 1, wherein the protease is expressed by one or both of Bacteroides vulgatus and Bacteroides dorei optionally as listed in Table 4.

7. The method of claim 1, further comprising administering to the subject an effective amount of a non-specific immunosuppressive agent, optionally selected from a seroid or a thiopurine.

8. The method of claim 1, wherein the subject has one or more of:
a high level of a protease expressed by the pathogenic bacteria;
a high activity of a protease expressed by the pathogenic bacteria;
a high level of a peptide, optionally selected from a dipeptide or an oligopeptide, optionally wherein the peptide is selected from a target of the protease or a fragment thereof and/or wherein the target is selected from one or more of a collagen, a mucin or a peptide identified in FIG. 11c or 12f;
an altered expression of one or more of protein(s) as identified in Table 7, optionally in a sample and further optionally in exosomes of a sample; such as a fecal sample and/or a fecal exosome;
an altered expression of a tight junction protein;
a decrease in epithelial cell circularity;
an increased permeability of an epithelial cell layer; or
resistance to a conventional treatment, optionally selected from a non-specific immunosuppressive agent.

9. (canceled)

10. (canceled)

11. The method of claim 10, wherein the protease inhibitor is administered locally or systemically and further wherein the inhibitor is one or both of a serine inhibitor or a cysteine inhibitor, optionally selected from one or more of: AEBSF, E-64, or Roche complete EDTA-free protease inhibitor cocktail.

12. The method of claim 11, herein the protease inhibitor is administered orally to the subject.

13. The method of claim 12, wherein the protease inhibitor is formulated in a pharmaceutical acceptable carrier and/or in a dosage form selected from the group consisting of: suppository, fecal transplant, within a biocompatible scaffold, powder, liquid, capsule, chewable tablet, swallowable tablet, buccal tablet, troche, lozenge, soft chew, solution, suspension, spray, tincture, decoction, infusion, and combinations thereof.

14. The method of claim 13, further comprising assaying a sample, optionally a fecal sample, isolated from the subject for one or more of the following:
a level of a protease expressed by the pathogenic bacteria;
a protease activity;
a level of a peptide optionally selected from a dipeptide or an oligopeptide, optionally wherein the peptide is selected from a target of the protease or a fragment thereof, and/or wherein the target is selected from one or more of a collagen, a mucin or a peptide identified in FIG. 11c or 12f;
expression of one or more of protein(s) as identified in Table 7, optionally in a sample and further optionally in exosomes of a sample, such as a fecal sample and/or a fecal exosome;
expression of a tight junction protein;
epithelial cell circularity; or
permeability of an epithelial cell layer.

15. A kit comprising an effective amount of a protease inhibitor, that targets a protease expressed by a pathogenic bacterium for use in the method of claim 1.

16. The kit of claim 15, further comprising an effective amount of a non-specific immunosuppressive agent, and wherein the inhibitor is one or both of a serine inhibitor or a cysteine inhibitor, optionally selected from one or more of: AEBSF, E-64, or Roche cOmplete EDTA-free protease inhibitor cocktail.

17. (canceled)

18. A method to identify a subject for protease therapy, comprising assaying a sample, optionally a fecal sample, isolated from the subject for one or more of the following:
a level of protease expressed by the pathogenic bacteria;
a protease activity;
a level of a peptide optionally selected from a dipeptide or an oligopeptide, or wherein the peptide is selected from a target of the protease or a fragment thereof, and/or wherein the target is selected from one or more of a collagen, a mucin or a peptide identified in FIG. 11c or 12f;
expression of one or more of protein(s) as identified in Table 7, optionally in a sample and further optionally in exosomes of a sample, such as a fecal sample and/or a fecal exosome;
expression of a tight junction protein;
epithelial cell circularity; or
permeability of an epithelial cell layer,
wherein one or more of the following identifies the patient for protease therapy: higher than normal levels of any one or more of the protease, the protease activity, or the peptides;
an altered expression of one or more of protein(s) as identified in Table 7, optionally in a sample and further optionally in exosomes of a sample, such as a fecal sample and/or a fecal exosome;
an altered expression of a tight junction protein;
a decrease in epithelial cell circularity; or
increased permeability of an epithelial cell layer.

19. The method of claim 18, further comprising one or both of the following:
administering to the subject an effective amount a protease inhibitor to the identified subject, or
to the subject administering an effective amount of a non-specific immunosuppressive agent to the subject.

20. A pharmaceutical composition comprising a protease inhibitor, an optional stabilizer, an optional preservative and a pharmaceutically acceptable carrier wherein the inhibitor is one or both of a serine inhibitor or a cysteine inhibitor, optionally selected from one or more of: AEBSF, E-64, or Roche complete EDTA-free protease inhibitor cocktail

21. (canceled)
